(19) United States
(12) Patent Application Publication
(10) Pub. No.: US 2025/0259750 A1
(43) Pub. Date: Aug. 14, 2025

(54) MULTI-MODAL MACHINE LEARNING TO DETERMINE RISK STRATIFICATION

(71) Applicants: Memorial Sloan-Kettering Cancer Center, New York, NY (US); Memorial Hospital for Cancer and Allied Diseases, New York, NY (US); Sloan-Kettering Institute for Cancer Research, New York, NY (US)

(72) Inventors: Emily Aherne, New York, NY (US); Kevin Boehm, New York, NY (US); Yulia Lakhman, New York, NY (US); Ines Nikolovski, New York, NY (US); Dmitriy Zamarin, New York, NY (US); Lora Ellenson, New York, NY (US); Druv Patel, New York, NY (US); Jianjiong Gao, New York, NY (US); Sohrab P. Shah, New York, NY (US); Ignacio Vazquez Garcia, New York, NY (US)

(21) Appl. No.: 18/856,807

(22) PCT Filed: Apr. 14, 2023
(86) PCT No.: PCT/US2023/018678

§ 371 Date: Oct. 14, 2024

Related U.S. Application Data
(60) Provisional application No. 63/331,390, filed on Apr. 15, 2022.

(51) Int. Cl. G16H 50/30; G06T 7/00; G16B 20/20

(52) U.S. Cl. CPC G16H 50/30; G06T 7/0012; G16B 20/20; G06T 2207/20084; G06T 2207/30024

(57) ABSTRACT

Presented herein are systems, methods, and non-transient computer readable media for determining risk scores using multimodal feature sets. A computing system may identify a first feature set for a first subject at risk of a condition. The first feature set may include (i) a first radiological feature derived from a tomogram of a section associated with the condition within the first subject, (ii) a first histologic feature acquired using a whole slide image of a sample having the condition from the first subject, and (iii) a first genomic feature obtained from gene sequencing of the first subject for genes associated with the condition. The computing system may apply the first feature set to a model. The computing system may determine, from applying the first feature set to the model, a predicted risk score of the condition for the first subject.

### CROSS-REFERENCE TO RELATED PATENT APPLICATIONS

The present application claims priority to U.S. Provisional Patent Application No. 63/331,390, titled “Multi-Modal Machine Learning to Determine Risk Stratification,” filed Apr. 15, 2022, which is incorporated herein by reference in its entirety.

### BACKGROUND

A computing system may apply various machine learning (ML) techniques on an input to generate an output.

### SUMMARY

Aspects of the present disclosure are directed to systems and methods of determining risk scores using multimodal feature sets. A computing system may identify a first feature set for a first subject at risk of a condition. The first feature set may include (i) a first radiological feature derived from a tomogram of a section associated with the condition within the first subject, (ii) a first histologic feature acquired using a whole slide image of a sample having the condition from the first subject, and (iii) a first genomic feature obtained from gene sequencing of the first subject for genes associated with the condition. The computing system may apply the first feature set to a model. The model may be established using a plurality of second feature sets and a plurality of expected risk scores for a corresponding plurality of second subjects. The computing system may determine, from applying the first feature set to the model, a predicted risk score of the condition for the first subject. The computing system may store, using one or more data structures, an association between the predicted risk score and the first feature set for the first subject.

In some embodiments, the computing system may classify the first subject into one of a plurality of risk level groups based on a comparison between the predicted risk score indicating a likelihood of an occurrence of an event due to the condition in the first subject and a threshold for each of the plurality of risk level groups. In some embodiments, the computing system may establish the model comprising a multivariate model using one or more features selected from the plurality of second feature set using one or more corresponding univariate models. In some embodiments, the computing system may provide information based on the association between the predicted risk score and the first feature set for the first subject.

In some embodiments, the computing system may determine a survival function identifying the predicted risk score for the first subject over a period of time. In some embodiments, the computing system may select, from a plurality of radiological features, the first radiological feature based on a hazard ratio of each of the plurality of radiological features determined using a univariate model for radiological features. In some embodiments, the computing system may select, from a plurality of histological features, the first histological feature based on a hazard radio of each of the plurality of histological features determined using a univariate model for histological features.

In some embodiments, the first radiological feature may be derived from the tomogram using a Coif-wavelet transform, and comprises at least one of: (i) a gray level co-occurrence matrix (GLCM), (ii) gray level dependence matrix (GLDM), (iii) a gray level run length matrix (GLRLM), (vi) a gray level size zone matrix (GLSZM), or (v) a neighboring gray tone difference matrix. In some embodiments, the first histologic feature further comprises at least one of: (i) a tissue type of the sample from which the whole slide image is derived, (ii) an area of cell nuclei corresponding to the condition within the sample, or (iii) a length of a portion of the sample corresponding to the tissue type.

### BRIEF DESCRIPTION OF THE DRAWINGS

FIG. 1. Schematic outline of the architecture. (a) Multiple data modalities were acquired through routine diagnostics to inform clinical decision making: (b) pre-treatment contrast-enhanced CT (CE-CT) scans of the abdomen and pelvis, (c) pre-treatment H&E-stained diagnostic biopsies, and (d) HRD status inferred from hybridization-capture based targeted sequencing or clinical HRD-DDR gene panels. (e) Integrated multimodal analyses by late fusion to stratify patients by overall survival. (Abbreviation: CT: computed tomography, GLSZM-SAE: gray level size zone matrix small area emphasis, GLRLM-GLV: gray level run length matrix gray level variance, H&E: hematoxylin and eosin, Var: variance, Nuc: nuclear, NGS: next-generation sequencing, LSTs: large-scale state transitions, NtAI: number of subchromosomal regions with allelic imbalance extending to the telomere, LOH: loss of heterozygosity, HRD: homologous recombination deficiency, CRS: chemotherapy response score, OS: overall survival).

FIG. 2(a)-(c). Overview of cohorts and data types acquired. (a) Venn diagram of patients in the training cohort with available clinical imaging and inferred HRD status. (b) Inferred subtypes, sequencing modality, dataset of origin, genes with five or more variants, and signature 3 status of each patient. Gray represents sequenced genes without the aberrations shown, and white represents an unsequenced gene. (c) Kaplan-Meier analysis on overall survival stratified by HRD status (N=377 patients). P-values were calculated using the log-rank test. (Abbreviation: Sig.: mutational signature, SNV: simple nucleotide variation, Amp.: copy number amplification, WES: whole-exome sequencing).

FIG. 3(a)-(h). High-autocorrelation omental implants are associated with shorter overall survival. (a) Segmented omental lesion (red) on CE-CT. (b) The log hazard ratio is depicted for each radiomic feature derived from omental implants (N=600 features). Features above the line were statistically significant by Cox regression after multiple testing correction of interquartile range-filtered features. (c) Adnexal radiomic features (N=600 features) were not significant by Cox regression after correction of interquartile range-filtered features. (d) The hazard ratio with 95% C.I. as estimated by Cox regression is shown for the feature in the final model, the autocorrelation derived from the gray level cooccurrence matrix for the wavelet-filtered image. (e) The value of this feature against OS is plotted for patients in the training set (N=251 patients). (f) Training and test concordance indices for the model along is shown: the height of each bar shows the c-Index, and the lower and upper points of the respective error bars depict the 95% C.I. by 100-fold leave-one-out bootstrapping. (g, h) Two risk groups based on the model's predicted risk score are shown for the training and test sets. P-values were derived using the log-rank test. (Abbreviation: glcm: gray level co-occurrence matrix, gldm: gray level dependence matrix, glrlm: gray level run length matrix, glszm: gray level size zone matrix, ngtdm: neighboring gray tone difference matrix, HLL: high-low-low wavelet filter, OS: overall survival, c: Harrell's concordance index).

FIG. 4(a)-(d). Weakly supervised deep learning accurately infers HGSOC tissue type on H&E. (a) Annotated tiles normalized using Macenko's method chosen at random. The number of tiles for each tissue type are shown. (b) Workflow of ResNet-18 model trained using the annotated regions. (c) Example of the model's predictions for an annotated region. (d) The confusion matrix aggregated across folds of cross validation for each of the tissue classes.

FIG. 5(a)-(g). Interpretable histopathologic features stratify HGSOC patients by OS. (a) Tissue map from H&E slides with nuclear detections yielding tissue-type and cell-type features (b) Log hazard ratios of the two chosen histologic features (with 95% C.I. as estimated by Cox regression; fit on N=243 patients). (c) Training and test concordance indices are shown: the height of each bar shows the c-Index, and the lower and upper points of the respective error bars depict the 95% C.I. by 100-fold leave-one-out bootstrapping. (d) Kaplan Meier survival analysis and log-rank test statistics for training (d) and test sets (e). (f, g) H&E of extreme examples of the model's inferred mean tumoral nuclear area (scale bar is 50 μm for each image).

FIG. 6(a)-(h). Multimodal integration improves stratification and identifies clinically significant subgroups. (a) The test c-indices for integration of combinations of multimodal features is shown: the height of each bar shows the c-Index, and the lower and upper points of the respective error bars depict the 95% C.I. by 100-fold leave-one-out bootstrapping. Asterisks denote 95% confidence of significant ordering of the test set by 1000-fold permutation test. (b) Log hazard ratios of imaging without (top) and with (bottom) HRD integration. Two modalities are shown in the top panel (fit on N=122 patients), and three are shown in the bottom (fit on N=114 patients). (c) Kaplan-Meier plot comparing high- and low-risk groups determined by the GRH model on the training set. P-value calculated using the log-rank test. (d) Kaplan-Meier plot comparing high- and low-risk groups test set. P-value calculated using the log-rank test. (e) Unique patients at risk of early death are identified by radiologic, histopathologic, and genomic modalities. Only patients in the test set with uncensored outcomes (N=23 patients) are shown. (f) Kendall rank correlation coefficient of the risk quantile across pairs of the individual modalities, indicating low mutual ordering information between individual modalities in the training set. (g) KM plot of GHR model risk groups on progression-free survival in the test set. (One patient has unknown PFS.) P-value calculated using the log-rank test. (h) Distributions of GHR model score of low (blue) and high (green) chemotherapy response score (CRS) in the training set (N=46 patients). Boxes denote interquartile range, with the center depicting the median and the whiskers denoting the entire distribution excluding any outliers. Significance was assessed by a one-sided Mann-Whitney U test: p=0.0044. ** denotes p<0.01. (Abbreviation: perm.: permutation test, G: genomic model, H: histopathologic model, R: radiologic model, C: clinical model, GHR: combined genomic histopathologic and radiologic model, GHRC: combined genomic histopathologic, radiologic, and clinical model, NET: no evidence of tumor, PFS: progression-free survival, OS: overall survival).

FIG. 7. Segmenting radiologist and CT vendor in training and test sets. (a) The same three expert radiologists segmented the discovery and test cases. (b) The most common scanner vendors were General Electric and Siemens for both cohorts, with other vendors being less represented. The test set contained one scan acquired on an Imatron device.

FIG. 8. Genomic features of the training and test sets. (a) The distribution of large-scale state transitions in the discovery cohort is depicted. The threshold for LST_{high} versus LST_{low} may be set at 7 LSTs, which is lower than previously reported thresholds for whole-exome sequencing. This is because the cohort is a targeted gene panel, and LSTs occurring at the same rate will measure lower on targeted panels compared to more comprehensive sequencing. (b) Signature three was detected by SigMA as the dominant signature with high confidence (HC) and low confidence (LC) in a significant number of cases, and the next most prevalent was the clock signature. (c) The COSMIC SBS3 frequencies for all TCGA-OV cases with sequencing from are shown, and the distribution is clearly bimodal but imbalanced. (d, e) Patients with HRD-type disease have longer OS than those with HRP-type disease in the training and test sets. (f) Incorporating thresholded LST counts as indicators of HRD status worsened the significance of the separation of the HRD and HRP curves and was thus not used in the definition of HRD status. (g) Using BRCA2 SNVs, BRCA1 SNVs, CCNE1 CNAs, and CDK12 SNVs, a subset of all patients were categorized into the following mutational subtypes: HRD-Deletion (HRD-DEL), HRD-Duplication (HRD-DUP), Foldback Inversion (FBI), and Tandem Duplications (TD), respectively. The patients stratify as expected by PFS, with HRP-type patients suffering earlier progression of disease (p value for log-rank test between aggregated HRD patients and aggregated HRP patients). (h) The stratification is ordered as expected but fails to reach significance for OS. (i) Using only patients with explicit evidence of HRP or HRD disease also yields groups with significantly different OS.

FIG. 9. Radiomic feature values by segmenting radiologist, CT scanner, and site. The radiomic feature chosen for the model is not confounded by (a) segmenting radiologist, (b) CT vendor, or (c) whether the scan was acquired at the institution or elsewhere.

FIG. 10. Example cross-validation histopathologic tissue type classifications.

FIG. 11. Histopathologic feature discovery. The logarithm of the univariate hazard ratio is depicted for each histopathologic feature, with the cluster in the upper right quadrant being primarily features describing tumor nuclear diameter and size.

FIG. 12. Histopathologic embeddings by specimen size and histopathologic feature selection. The embeddings in UMAP space of the two-feature histopathologic signature do not appear influenced by the relative specimen size (here depicted as the quantile of the number of foreground tiles detected). The larger specimens appear relatively evenly distributed, with the exception of a preponderance of smaller specimens toward the bottom left of the plot.

FIG. 13. Test performance of histopathologic-radiomic model. (a) The RH model separates the high- and low-risk groups by OS, but with a reduced separation (45% and 70% survival at 36 months). (b) However, the RH model-determined curves do not separate significantly by PFS.

FIG. 14. Learning only from cases with full information (N=114) worsens performance. (a) Ovarian and (b) omental features do not reach significance during discovery. (c) Histopathologic feature discovery is similar. (d) Performance on the test set against overall survival is worse. (e) The GRH model fails to significantly stratify the test set by OS. (f) It also fails to significantly stratify the test set by PFS.

FIG. 15. No robust association exists between individual modalities in the test set. (a) The maximal magnitude of the Pearson correlation between individual modalities is 0.178. (b) The maximal magnitude of the Spearman correlation between individual modalities is 0.135.

FIG. 16. Chemotherapy response scores for all models on the test set. (a-o) for C, G, GC, GH, GHC, GR, GRC, GRH, GRHC, H, HC, R, RC, RH, and RHC models, respectively.

FIG. 17 depicts a block diagram of a system for determining risk scores using multimodal feature sets in accordance with an illustrative embodiment.

FIG. 18A depicts a block diagram of a process of extracting multimodal features in the system for determining risk scores in accordance with an illustrative embodiment.

FIG. 18B depicts a block diagram of a process of applying risk prediction models to multimodal features in accordance with an illustrative embodiment.

FIG. 19 depicts a flow diagram of a method of determining risk scores using multimodal feature sets in accordance with an illustrative embodiment.

FIG. 20 depicts a block diagram of a server system and a client computer system in accordance with an illustrative embodiment.

### DETAILED DESCRIPTION

Following below are more detailed descriptions of various concepts related to, and embodiments of, systems and methods for determining risk stratification using multi-modal machine learning models. It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes.

Section A describes multi-modal machine learning to improve risk stratification of high-grade serious ovarian cancer;

Section B describes systems and methods of determining risk scores using multimodal features; and

Section C describes a network environment and computing environment which may be useful for practicing various embodiments described herein.

### A. Multi-Modal Machine Learning to Improve Risk Stratification of High-Grade Serious Ovarian Cancer

Patients with high-grade serous ovarian cancer (HGSOC) suffer poor prognosis and variable response to treatment. Known prognostic factors for this disease include homologous recombination deficiency status, age, pathologic stage, and residual disease status after debulking surgery. Other approaches have highlighted important prognostic information captured in computed tomography and histopathologic specimens, which can be exploited through machine learning. However, little is known about the capacity of combining features from these disparate sources to improve prediction of treatment response. Here, a multimodal dataset of 444 patients with primarily late-stage HGSOC is assembled, and quantitative features, such as tumor nuclear size on H&E and omental texture on CE-CT, associated with prognosis are discovered. It was found that these features contributed complementary prognostic information relative to one another and clinico-genomic features. By fusing histopathologic, radiologic, and clinico-genomic machine learning models, a path toward improved risk stratification of cancer patients through multimodal data integration is demonstrated.

### Introduction

High-grade serous ovarian cancer (HGSOC) is the most common cause of death from gynecologic malignancies, with a five-year survival rate of less than 30% for metastatic disease. Initial clinical management relies on either primary debulking surgery (PDS), or neoadjuvant chemotherapy followed by interval debulking surgery (NACT-IDS). Endogenous mutational processes are an established determinant of clinical course, with improved response of homologous recombination deficient (HRD) disease to platinum-based chemotherapy and poly-ADP ribose polymerase (PARP) inhibitors. More nuanced genomic analyses integrating point mutation and structural variation patterns further refine this stratification into four biologically and prognostically meaningful subtypes including distinct sub-groups of HRD, foldback inversion enriched tumors and those with distinctive accrual of large tandem duplications. Beyond genomic factors, clinical indicators such as patient age, pathologic stage, and residual disease (RD) status after debulking surgery are also prognostic. However, these clinico-genomic factors alone fail to adequately account for the heterogeneity of clinical outcomes. Identifying patients at risk of poor response to standard treatment remains a critical unmet need. Improved risk stratification models would aid gynecologic oncologists in selecting primary treatment, planning surveillance frequency, making decisions about maintenance therapy, and counseling patients about clinical trials of investigative agents.

Beyond clinico-genomic features, multi-scale clinical imaging is routinely acquired during the course of care, including contrast-enhanced computed tomography (CE-CT) at the mesoscopic scale and hematoxylin and eosin (H&E)-stained slides at the microscopic scale. Digital forms of these diagnostics present opportunities to develop computational models and test whether integrating these data modalities improves identification of risk groups for HGSOC. At the mesoscopic scale, other radiologic studies have uncovered quantitative CE-CT features that are predictive of early progression, time to recurrence, and overall survival in HGSOC. Other approaches have analyzed the prognostic information captured within adnexal lesions or the whole burden of disease and variably use either deep learning or empirically reproducible radiomic features from the Imaging Biomarker Standardization Initiative. However, a radiomic prognostic model based on omental lesions has not yet been developed even though omental implants are ubiquitous in advanced-stage disease. Such a model would be advantageous because it is possible even for less experienced observers to delineate omental implants, and it would alleviate the need for highly challenging and time-consuming segmentation of the total burden of disease.

At the microscopic scale, H&E-stained tissue biopsies enable pathologic diagnosis and are routinely acquired before the start of therapy. A quantitative histopathologic study of HGSOC identified patterns of immune infiltration on H&E slides that correlate with mutational subtypes. In other cancer types, studies of whole slide images (WSIs) have advanced the ability to quantify the histopathologic architecture of tumors using deep and interpretable features. Apart from stage, HGSOC lacks independent pre-treatment pathologic factors by which to stratify patients, and quantitative approaches thus present an opportunity to systematically develop scaled models that are beyond qualitative human interpretation. Interpretable features are less prone to overfitting in small cohorts and can be more easily interrogated by human pathologists.

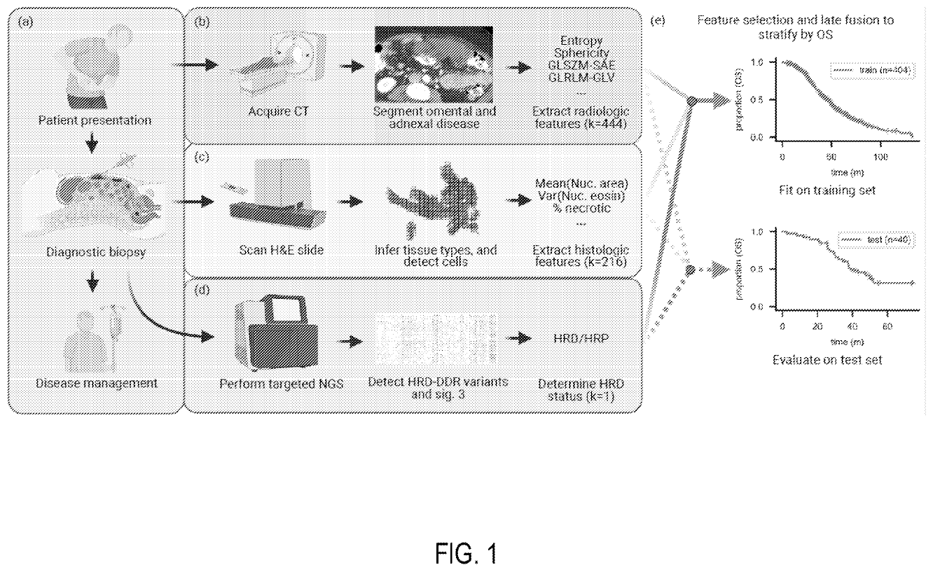

Conceptually, genomic sequencing does not account for spatial context, and it is thus hypothesized that multiscale imaging contains complementary information, rather than merely recapitulating genomic prognostication. There is also the potential for clinical multimodal machine learning to outperform unimodal systems by combining information from multiple routine data sources. In the present disclosure, the complementary prognostic information of multimodal features derived from clinical, genomic, histopathologic, and radiologic data obtained during the routine diagnostic workup of HGSOC patients is examined (FIG. 1a). The prognostic relevance of ovarian and omental radiomic features derived from CE-CT are tested, and a model based on omental features (FIG. 1b) and a histopathologic model based on pre-treatment tissue samples to risk stratify patients (FIG. 1c) are developed. The models were validated on a test cohort and integrated with clinical and genomic information (FIG. 1d) using a late fusion multimodal statistical framework (FIG. 1e). These results revealed the empirical advantages of cross-modal integration and demonstrated the ability of multimodal machine learning models to improve risk-stratification of HGSOC patients.

### Results

### Cohort and Clinical Characteristics

444 patients with HGSOC, including 296 patients treated at Memorial Sloan Kettering Cancer Center (MSKCC) and 148 TCGA-OV cases, were analyzed. The 40 test cases were randomly sampled from the entire pool of cases with all data modalities available for analysis; the remaining 404 cases were used for training. The training set contained 160 patients with stage IV disease, 225 with stage III, 10 with stage II, 8 with stage I, and 1 with unknown stage (Supplementary Table 1). The test cohort contained 31 stage IV and 9 stage III patients 23. Median age at diagnosis was 63 years [IQR 55-71] for the training set and 66 years [IQR 59-70] for the test set. In the training cohort, 175 patients received neoadjuvant chemotherapy followed by interval debulking surgery (NACT-IDS), and the remaining 82 underwent primary debulking surgery (PDS). In the test cohort, 31 received NACT-IDS and 8 underwent PDS. 61 MSKCC patients were known to have received PARP inhibitors (Supplementary Table 1). Treatment regimens are not annotated for the remaining 148 TCGA patients. Median OS was 38.7 months [IQR 25-55] for training patients and 37.6 months [IQR 26-49] for testing patients. 132 training patients and 17 testing patients had censored OS outcomes (Supplementary Table 2).

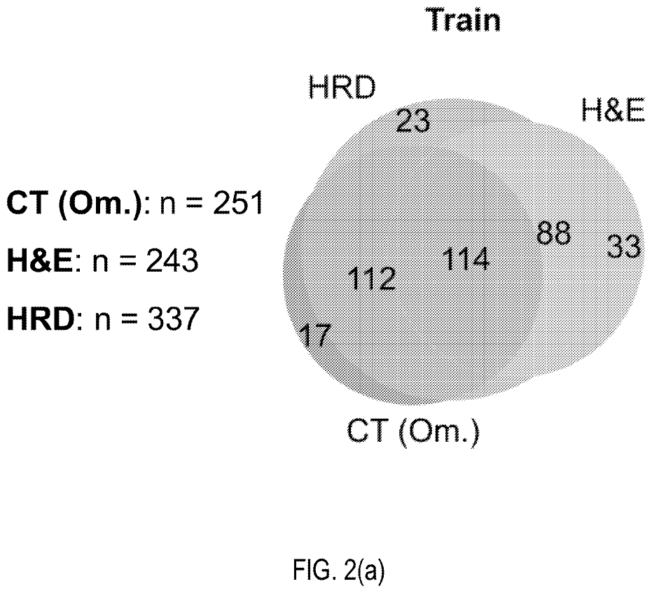

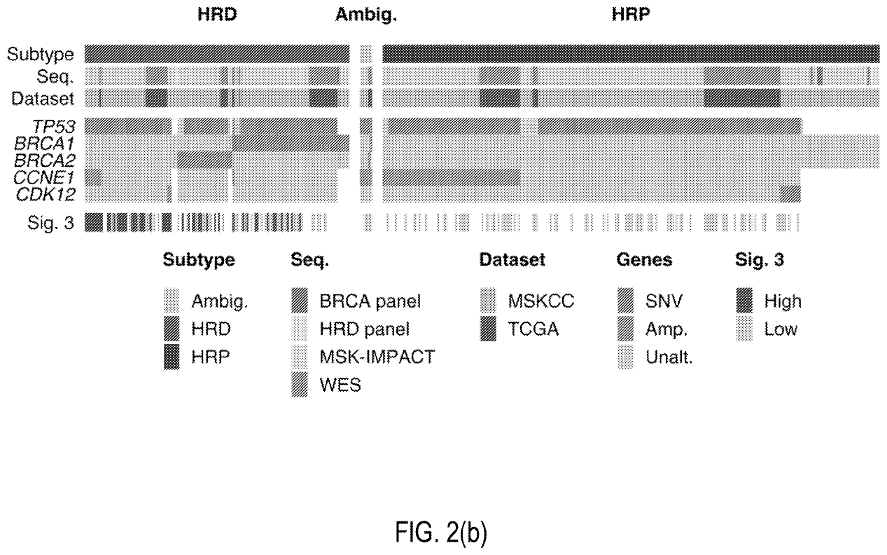

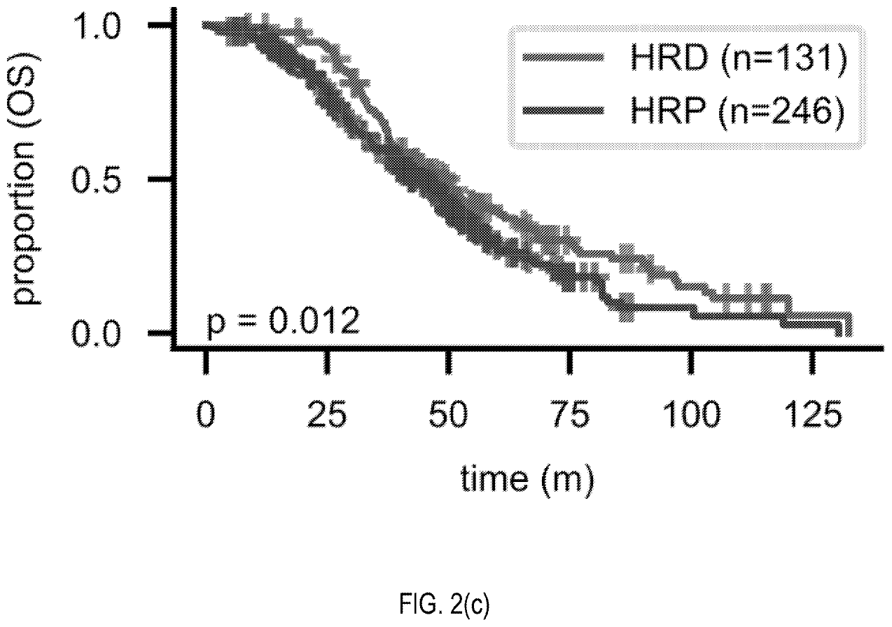

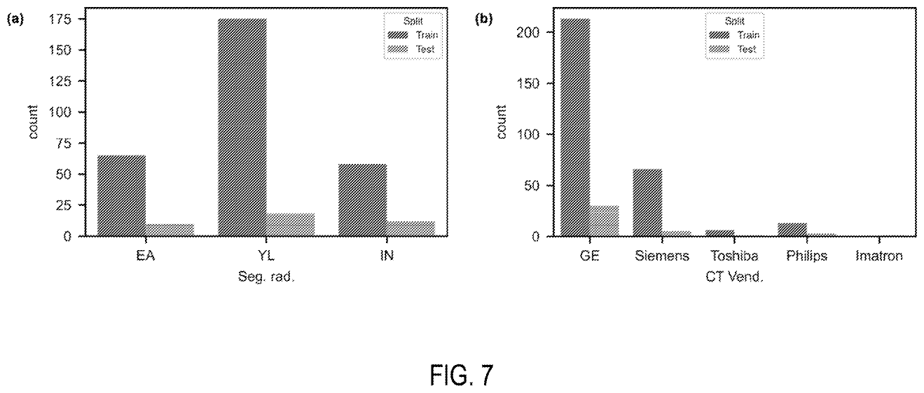

Among 404 patients in the training cohort, 243 patients had H&E WSIs, 245 patients had adnexal lesions on pretreatment CE-CT, and 251 patients had omental implants on pretreatment CE-CT (FIG. 2a). All 40 patients in the internal test cohort had omental lesions on CE-CT, H&E WSIs, and available sequencing by construction; 29 patients had ovarian lesions on CE-CT. Three gynecologic radiologists volumetrically segmented adnexal lesions and representative omental lesions on all sections containing these lesions (FIG. 7a). The training and testing data were acquired with similar CT scanners (FIG. 7b).

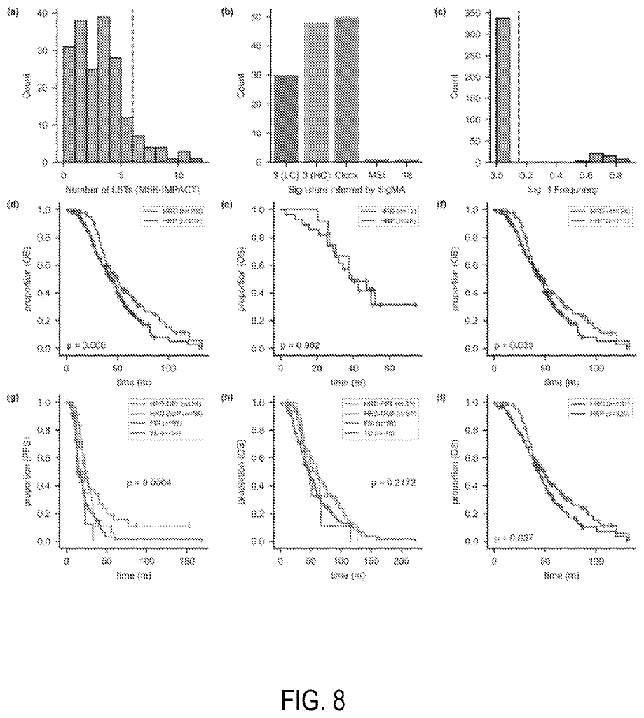

Clinical sequencing is used to infer HRD status, in particular variants in genes associated with HRD DNA damage response (DDR) such as BRCA1 and BRCA2, and those specific to disjoint tandem duplicator and foldback inversion-enriched mutational subtypes (CDK12 and CCNE1 respectively, FIG. 1d, FIG. 2b-c). The genomes of 130 patients with appropriate consent are examined for direct evidence of homologous recombination deficiency, namely COSMIC single base substitution (SBS) signature 3, which is associated with defective HRD-DDR. In this subset of MSKCC patients, signature 3 was detected by SigMA with high confidence in 48 cases, detected with low confidence in 30 cases, and found not to be the dominant signature in 52 cases (FIG. 8b). In the TCGA, signature 3 was high in 6 cases and low in 51 (FIG. 8c). Patients with available sequencing and without evidence for HRD or HRP (N=126) were treated as HRP. Patients with conflicting evidence (N=6) or without sequencing (N=61) were assigned a label of “ambiguous” and excluded from all analyses involving HRD status. In total, the training cohort contained 218 HRP and 119 HRD cases (FIG. 2c). The test set contained 12 HRD and 28 HRP cases. HRD status alone (excluding ambiguous) stratified patients by OS with a c-Index of 0.55 in the training cohort and 0.52 in the test set (without fitting any model parameters; FIG. 8d-e). Aberrations specific to distinct endogenous mutational processes also stratified patients as expected: that is, patients with HRP disease had worse outcomes than those with HRD disease (p=7e-3; FIG. 8g, 2i).

### CE-CT Imaging Feature Selection and Stratification

The prognostic relevance of features derived from radiology scans either obtained at the institution (91; 27%) using GE Medical Systems CT scanners or acquired at outside institutions (247; 73%) from a variety of CT scanners (FIG. 7; Supplementary Table 3) is studied. The majority of CE-CT scans were acquired with a peak kilovoltage of 120 (median 120 kVp, range: 90-140; Supplementary Table 3) and reconstructed with the standard convolutional kernel using 5 mm slice thickness (median 5 mm; range: 2.5-7.5; Supplementary Table 3). Three fellowship-trained radiologists with expertise in gynecologic oncologic imaging manually segmented all adnexal masses and representative omental implants on each pretreatment CE-CT scan (FIG. 1b, 3a).

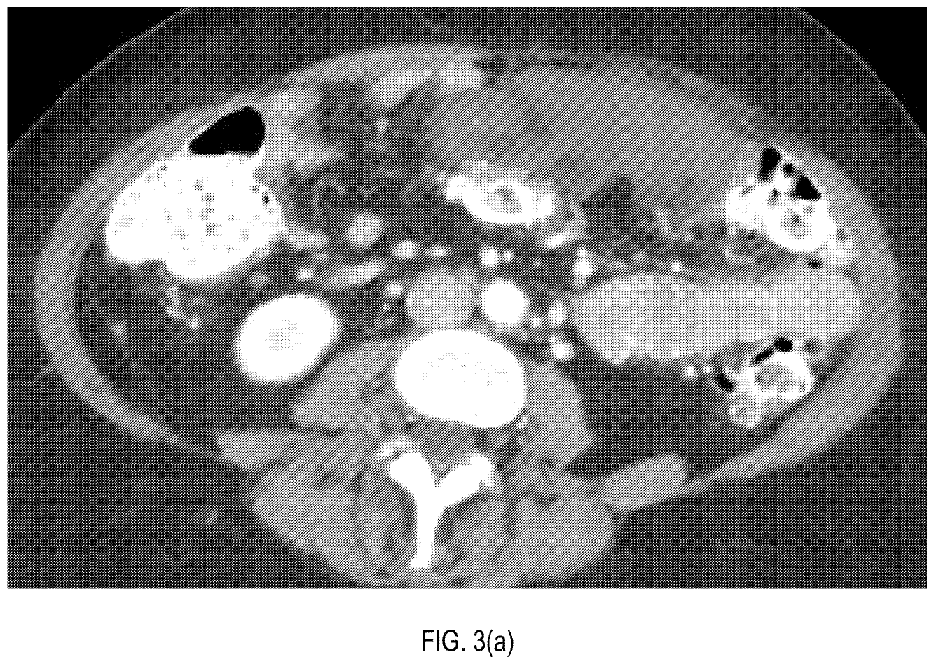

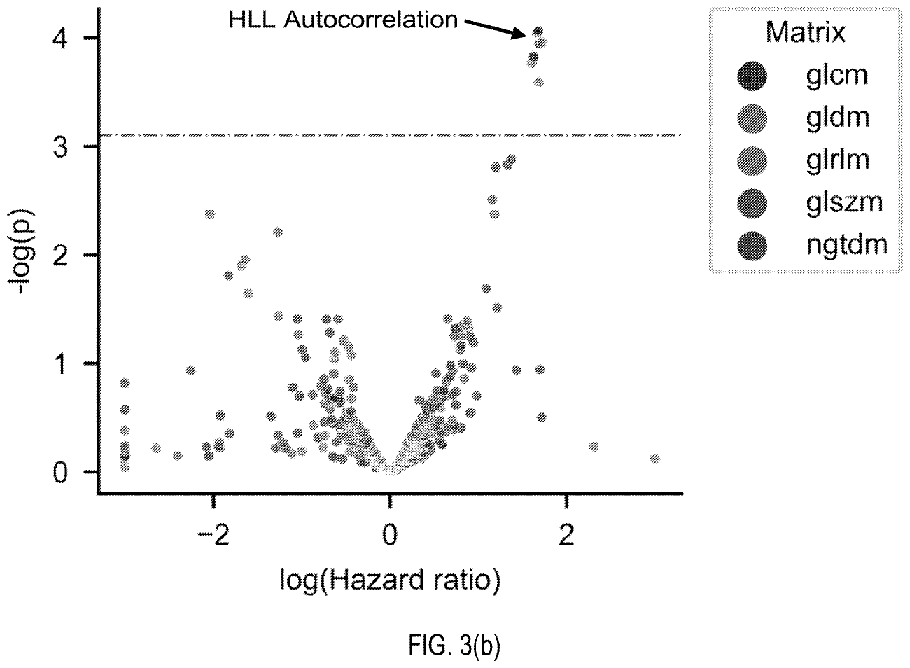

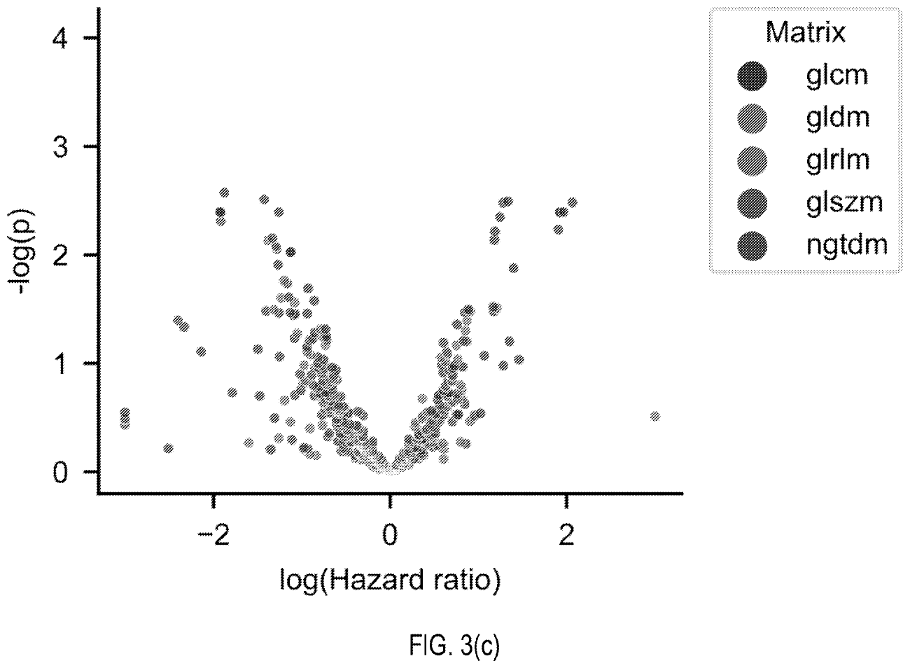

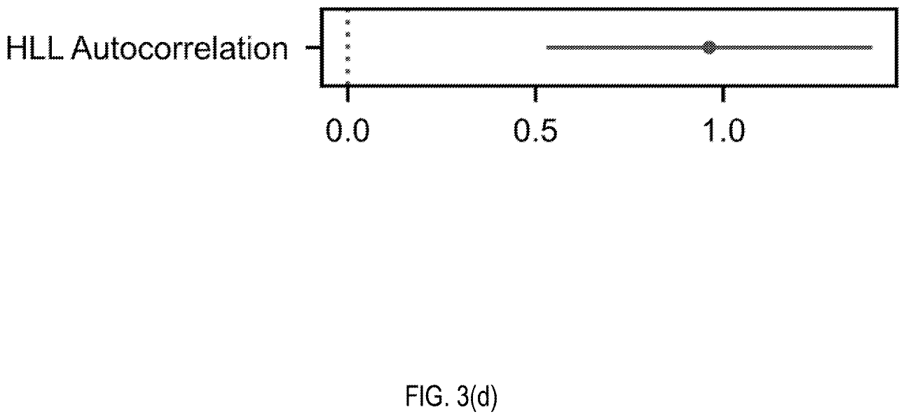

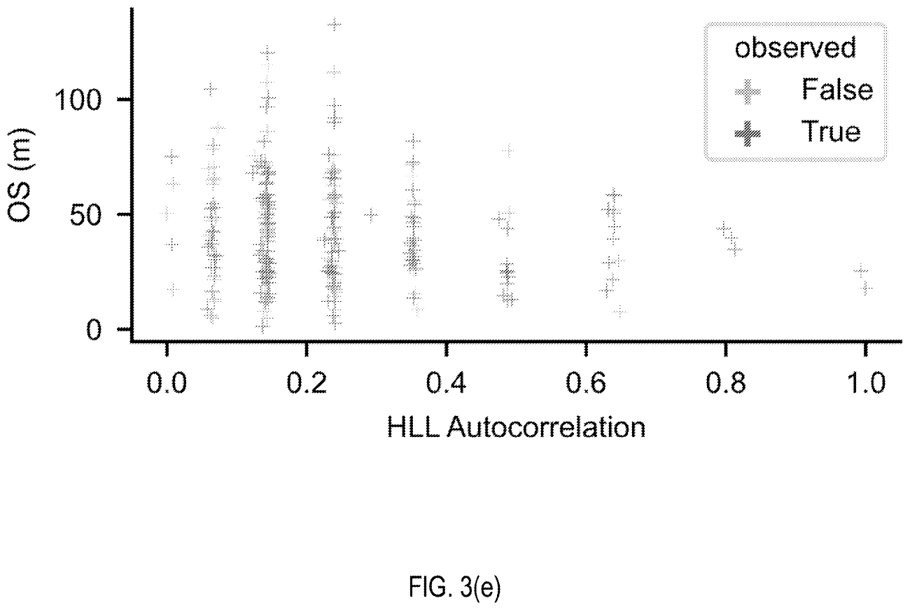

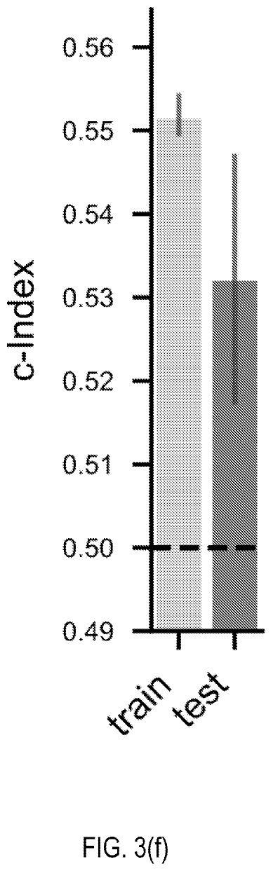

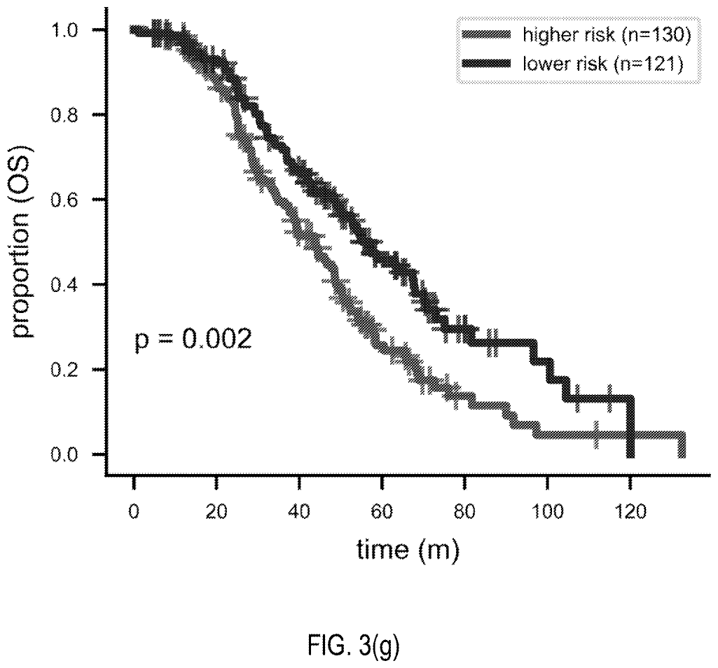

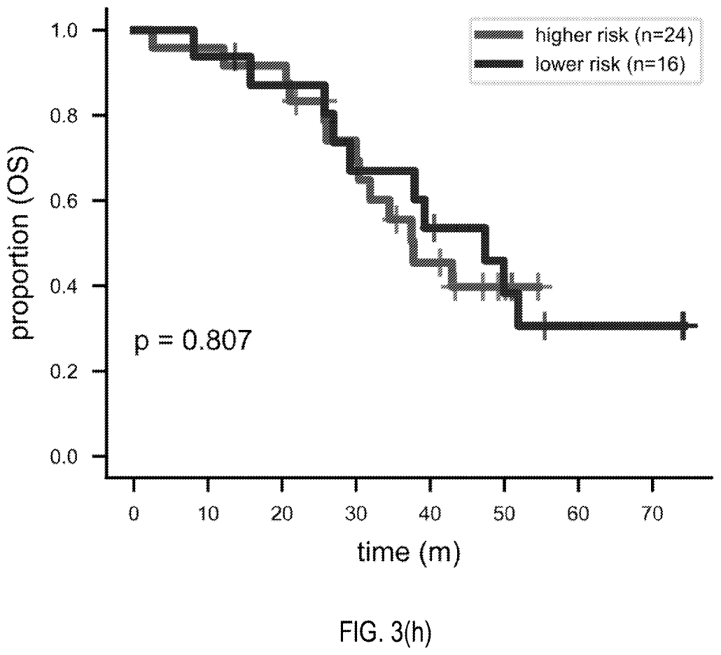

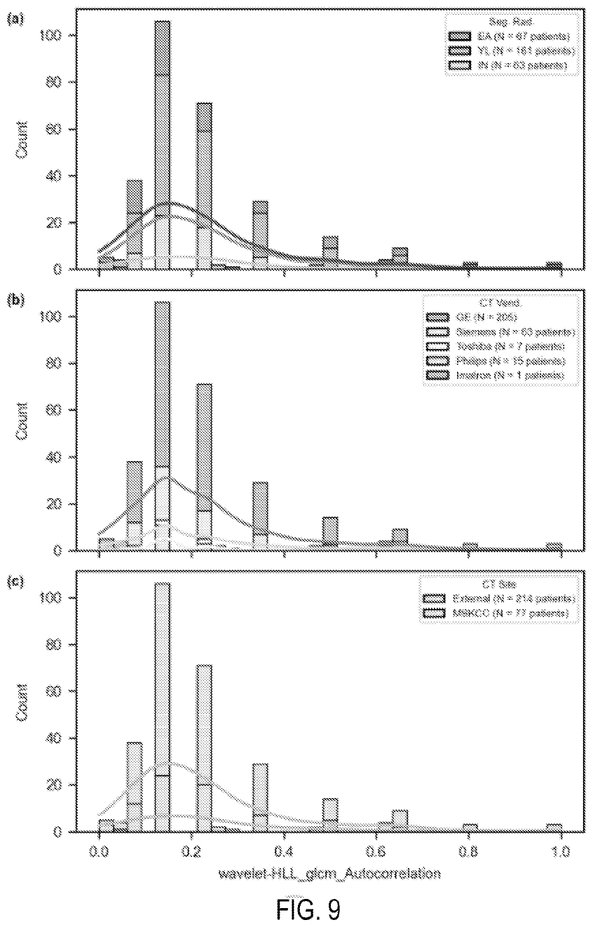

Radiomic features are extracted from Coif-wavelet transformed images, yielding a 444-dimensional radiomic vector per site per patient. Using the training cohort, the hazard ratios and prognostic significance of omental and ovarian radiomic features are calculated using univariate Cox proportional hazards models (Supplementary Table 4). After correction for multiple hypothesis testing, omental features (FIG. 3b) and none of the ovarian features exhibited statistically significant hazard ratios (FIG. 3c). Hence, going forward, the omental implants are only considered. Cox models are iteratively fit and pruned for multivariable significance on the nine omental features (Algorithm 1), yielding a univariate model based on the autocorrelation of the gray level co-occurrence matrix derived from the HLL Coif wavelet-transformed 29 images (FIG. 3d). This feature exhibited a log(HR) of 1.68 (corrected p<0.01; FIG. 3e) and was invariant to CT scanner manufacturers and segmenting radiologists (FIG. 9). The model stratified patients in the training and the test sets with concordance indices of 0.55 [95% C.I. 0.549-0.554] and 0.53 [95% C.I. 0.517-0.547], respectively (FIG. 3f). Kaplan-Meier analysis of the high- and low-risk groups (as determined by inferred risk) showed statistically different overall survival by the log-rank test (p<0.01) in the training set (FIG. 3g), with median survival of 44 and 57 months, respectively but not in the test set, with median survival of 38 and 47 months, respectively (FIG. 3h).

### Histopathologic Tissue Type Classifier for Interpretable Features

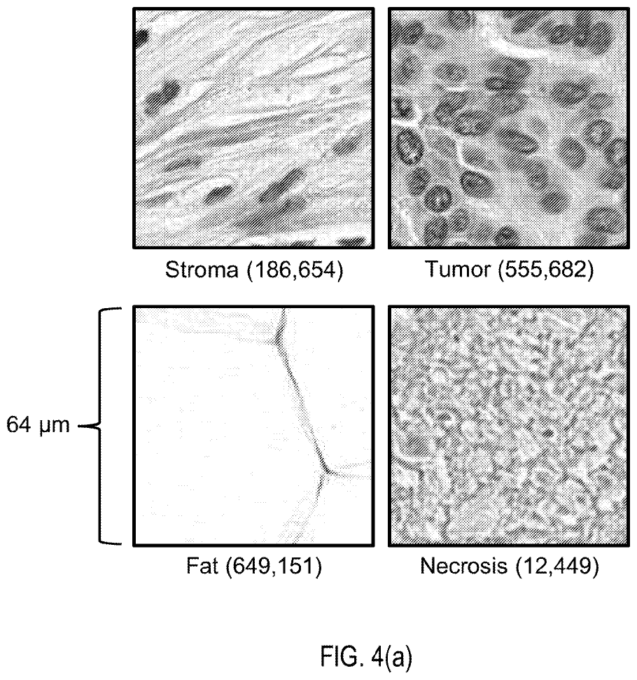

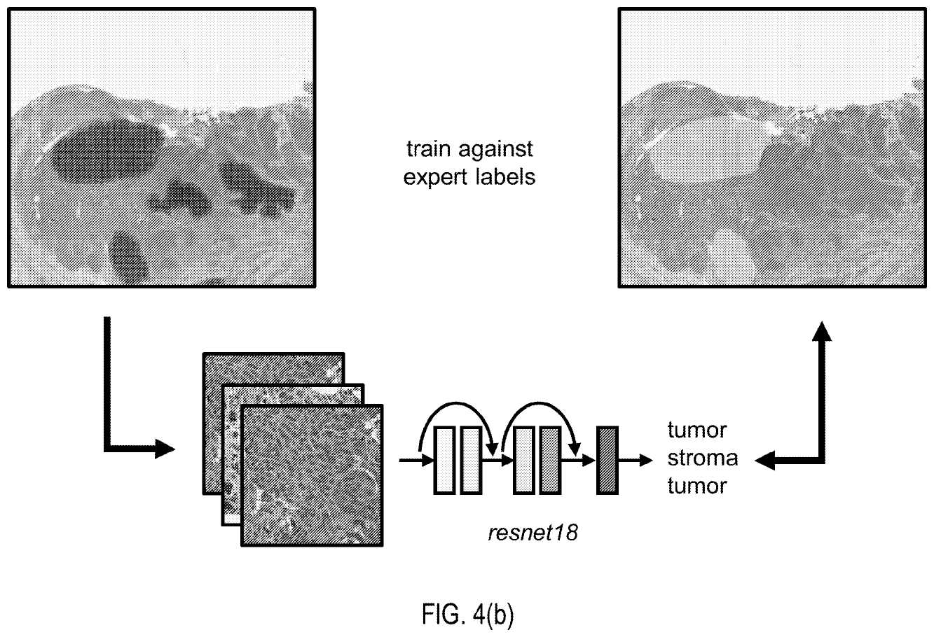

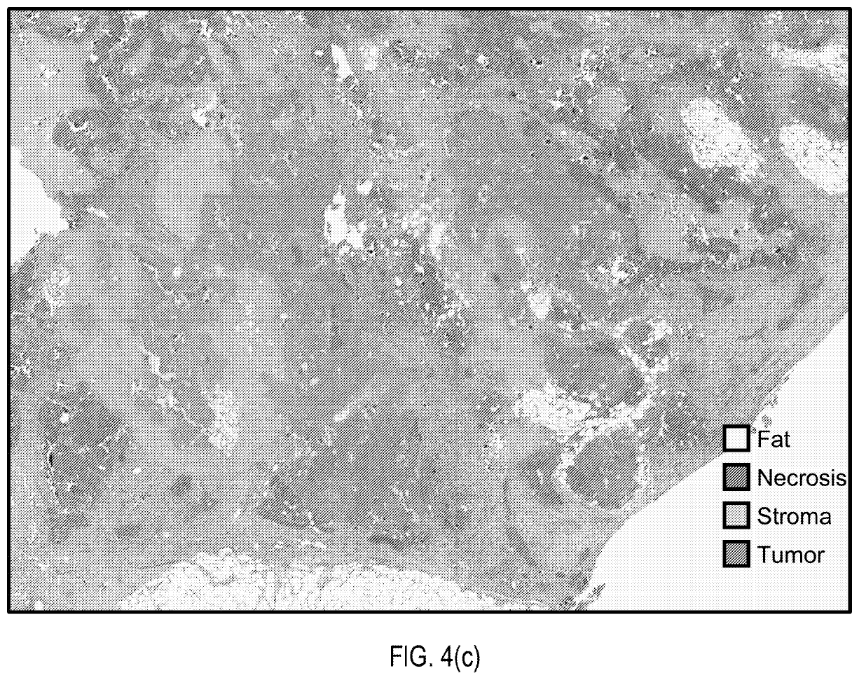

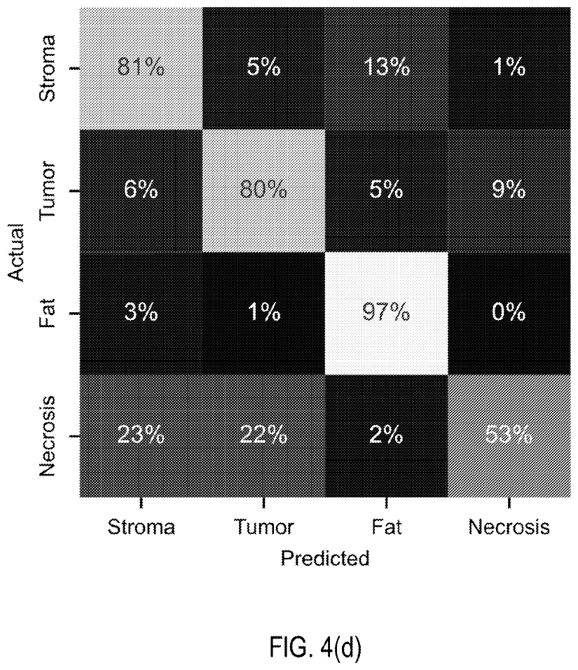

Next, a tissue type classifier is trained from histology images using a weakly supervised approach. Tissue types on 60 H&E WSIs are annotated, yielding more than 1.4 million partially overlapping tiles, each measuring 128×128 pixels (64×64 μm) and containing 4096 μm² of tissue (FIG. 4a). A ResNet-18 convolutional neural network (CNN) pretrained on ImageNet (FIG. 4b) classified tissue types with an accuracy of 0.88 (range 0.77-0.95) on pathologist-annotated areas labeled as fat, stroma, necrosis, and tumor (FIG. 4c) by four-fold slide-wise cross validation. Notably, the model correctly identified small regions of fat within stromal annotations and necrotic regions within the tumor, supporting the suitability of weakly supervised deep learning for this task and refining annotations into more granular classifications.

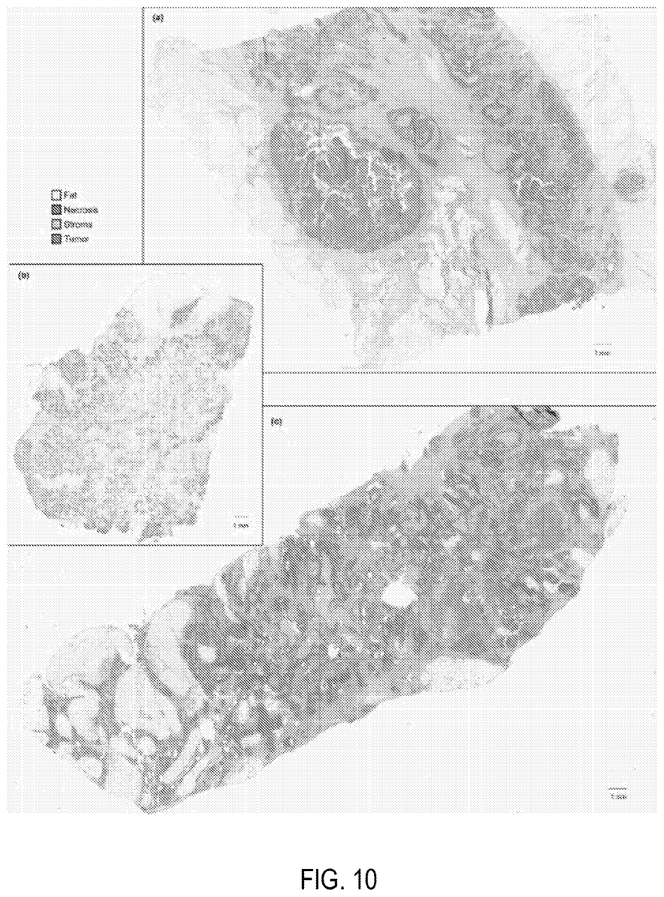

The cross-validation confusion matrix aggregated across folds showed good performance overall (FIG. 4d), with the most significant confusion being necrotic tiles predicted to be tumor and stroma. However, one disadvantage of weakly supervised learning is that neither the training data nor the validation data are exactly labeled. Hence, the cross-validation metrics are not computed against the exact truth. Visual inspection of the predictions were qualitatively concordant with only moderate confusion of necrosis with tumor and stroma (FIG. 10).

### Histopathologic Stratification

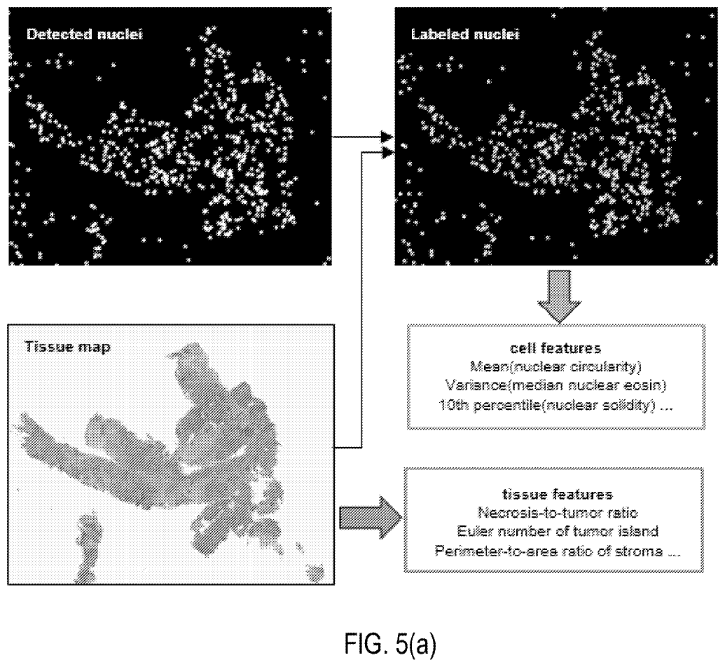

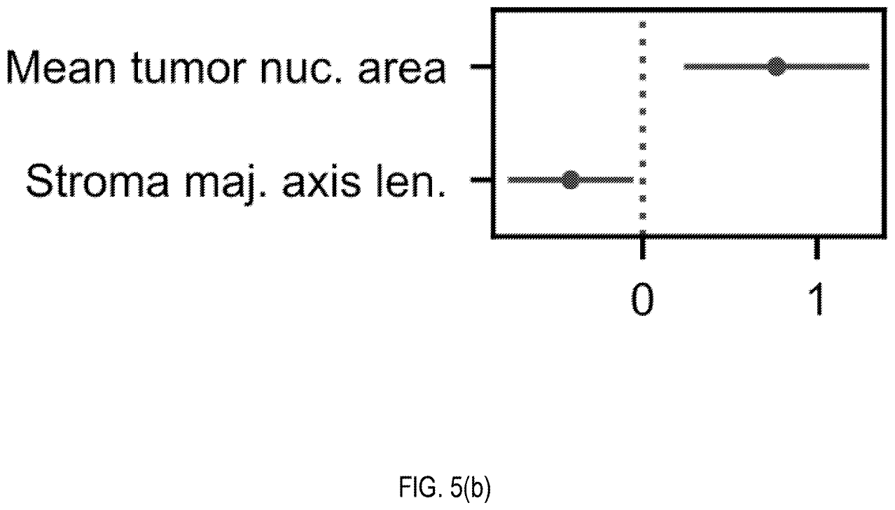

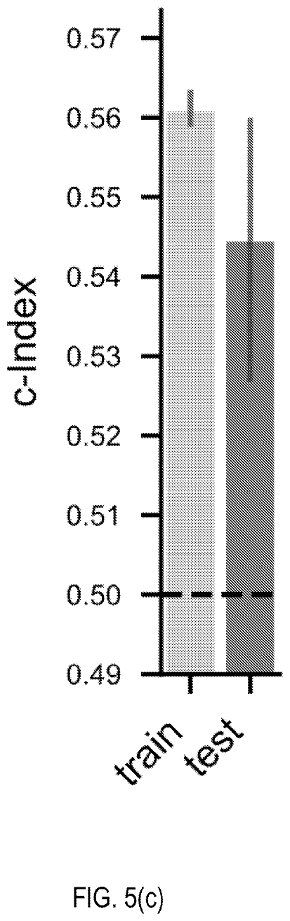

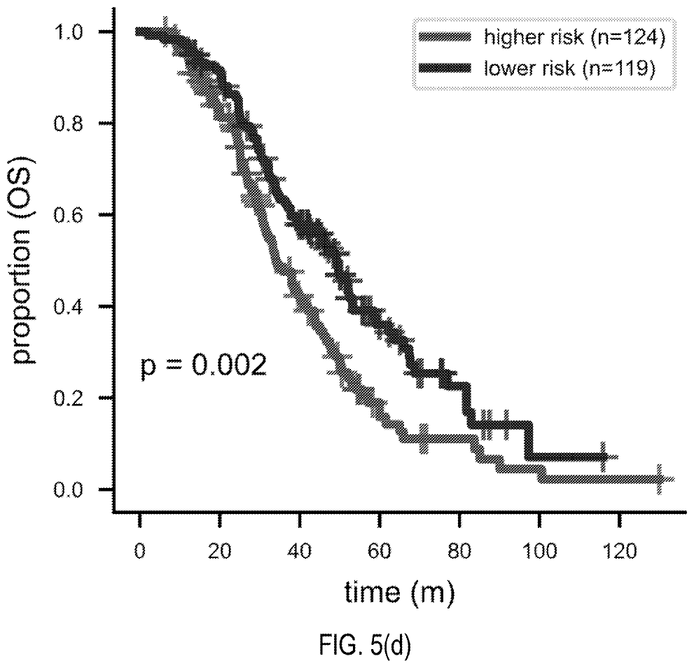

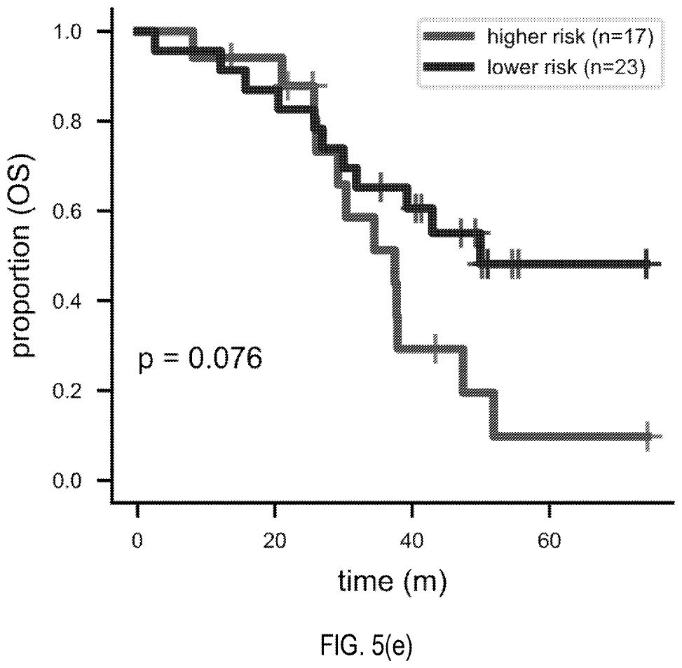

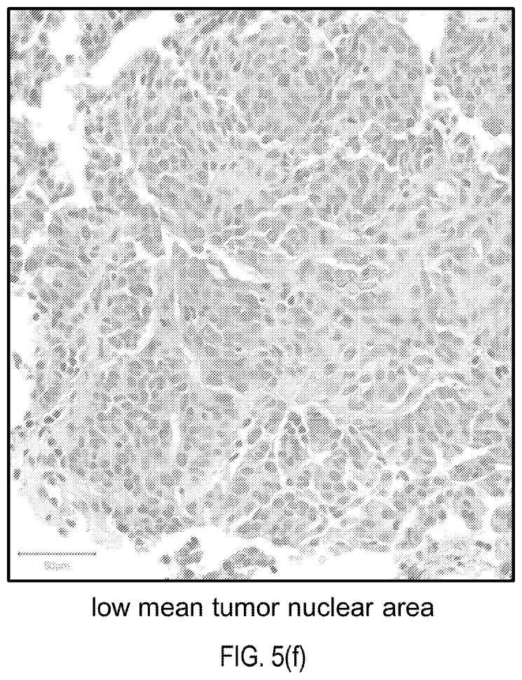

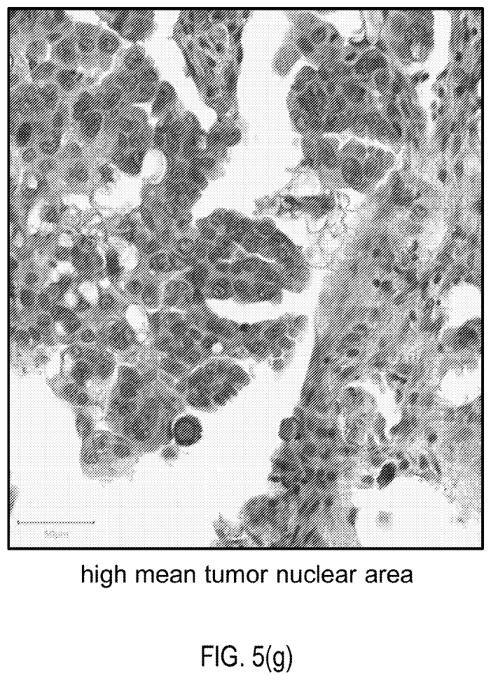

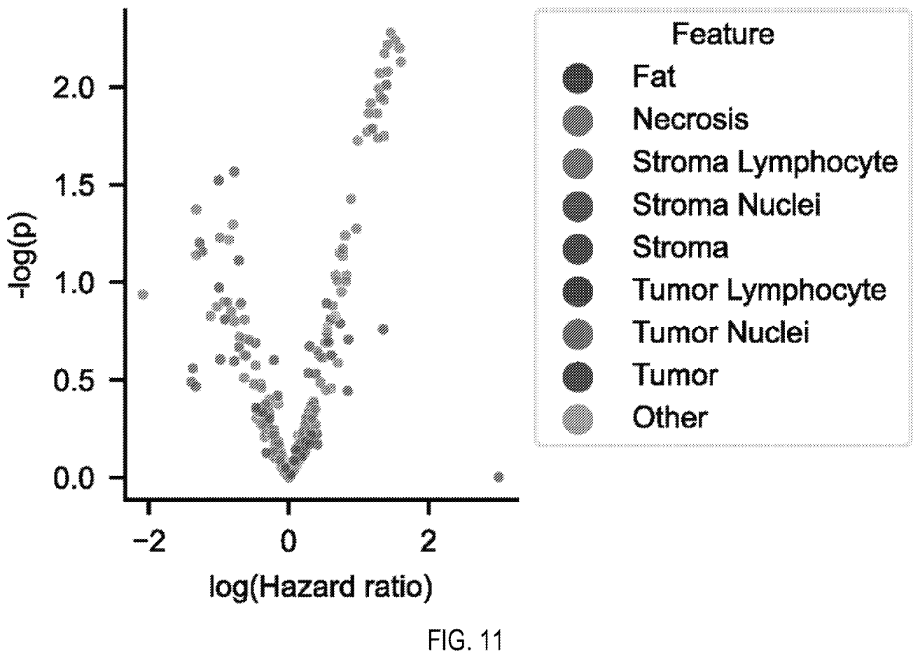

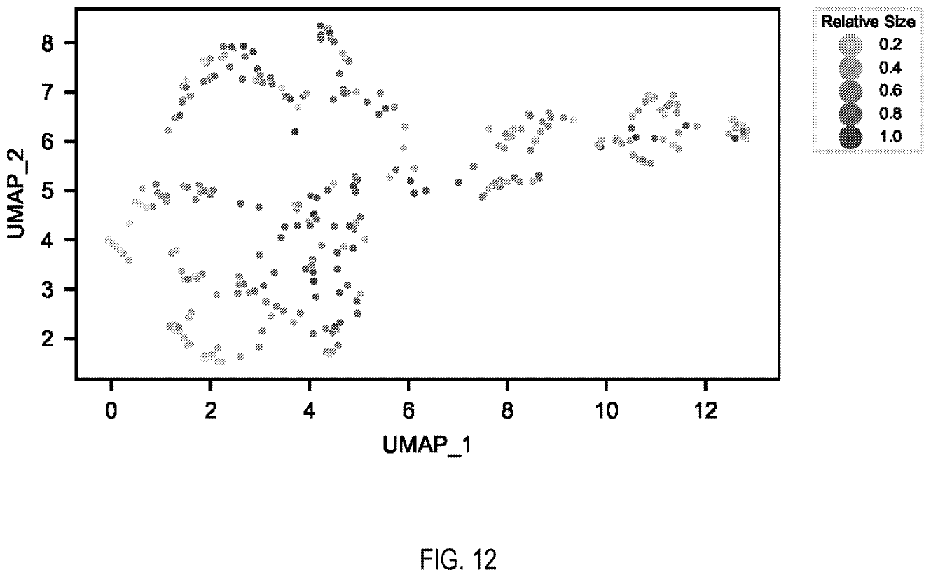

Tissue type classifier is applied to the 243 training H&E WSIs of lesions from pretreatment specimens (FIG. 1c). These inferred tissue type maps are combined with detected cellular nuclei, yielding labeled nuclei (FIG. 5a). Subsequently, cell-type features are extracted from these nuclei and tissue-type features from the tissue-type maps based on the methods. This yielded a histopathologic vector of 216 features. Next the hazard ratios of features are identified using univariate Cox models fit on slides in the training cohort. Several tissue-type features, such as overall tumoral area, were partially determined by specimen sizes, and were thus controlled for this during selection. Of the 24 features with a log(hazard ratio) found to be significantly different from 0 with 95% confidence, 20 related to tumor nuclear diameter or size, with larger being associated with shorter OS (FIG. 11; Supplementary Table 5). Again, Cox models were iteratively fit and pruned per Algorithm 1, yielding a multivariable model with two features: the mean tumor nuclear area and the major axis length of the stroma (FIG. 5b). This histopathologic signature was not confounded by specimen size (FIG. 12). This model stratified the training and test sets, with concordance indices of 0.56 [95% C.I. 0.559-0.564] and 0.54 [95% C.I. 0.527-0.560], respectively (FIG. 5c). High- and low-risk groups established based on the inferred risk scores separate well for the training set with median survival of 34 and 49 months, respectively (FIG. 5d; p<0.01). For the test set, the risk groups trended toward—but did not attain—significantly different separation, with median survival of 37 and 50 months (FIG. 5e; p=0.076). To probe the interpretability of the histopathologic features, the mean tumor nuclear area is investigated: examples of low (FIG. 5f) and high (FIG. 5g) values are shown, which were associated with better and worse prognosis, respectively.

### Multimodal Prognostication

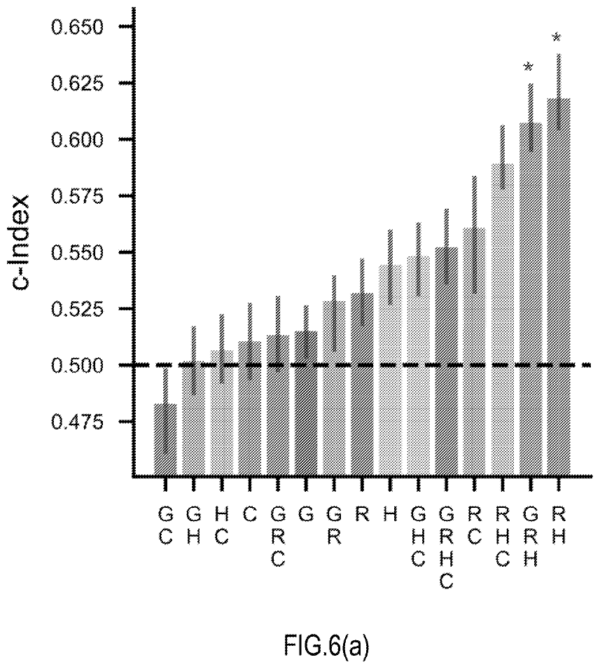

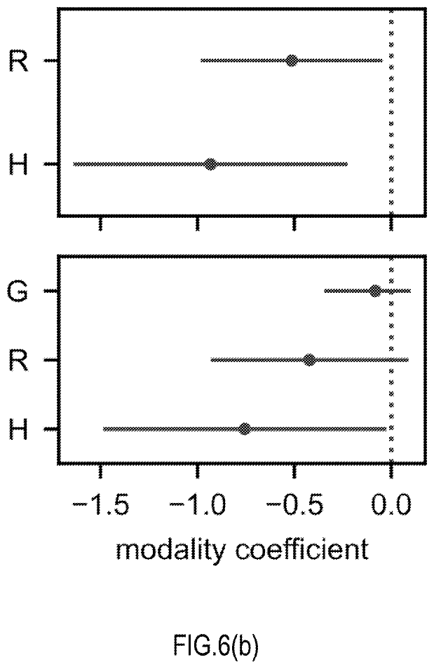

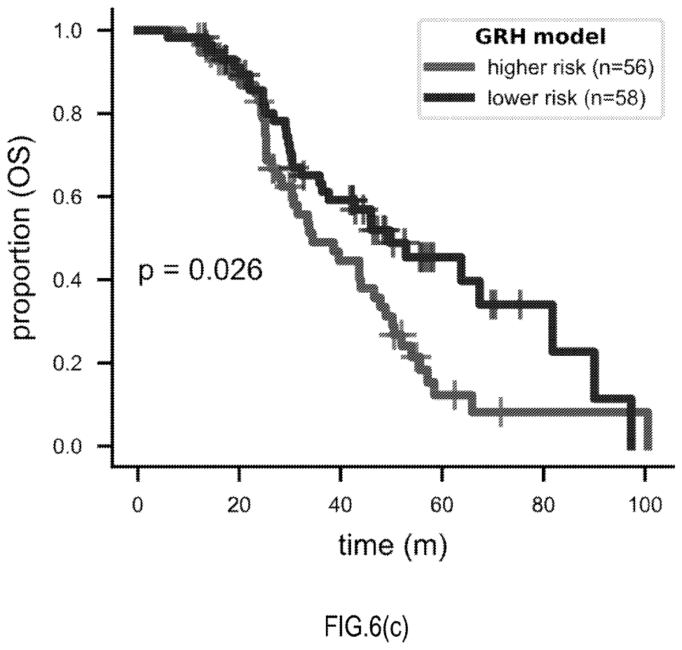

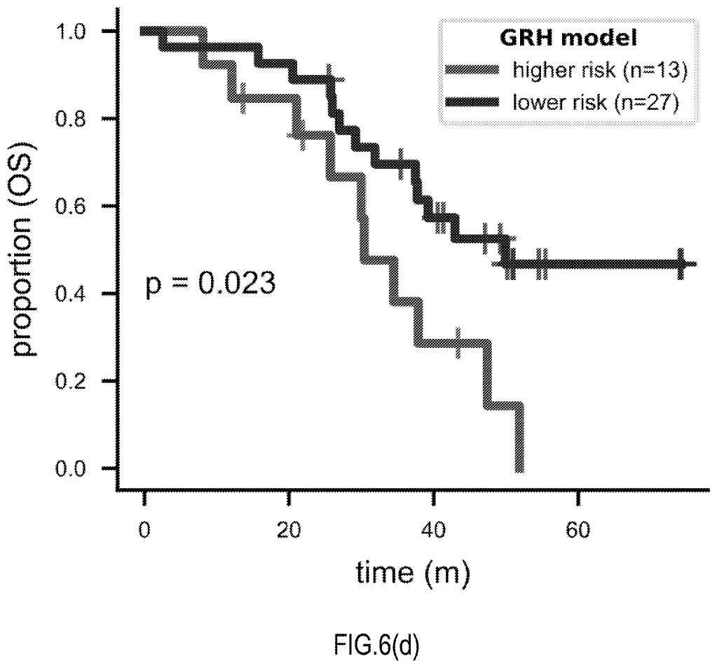

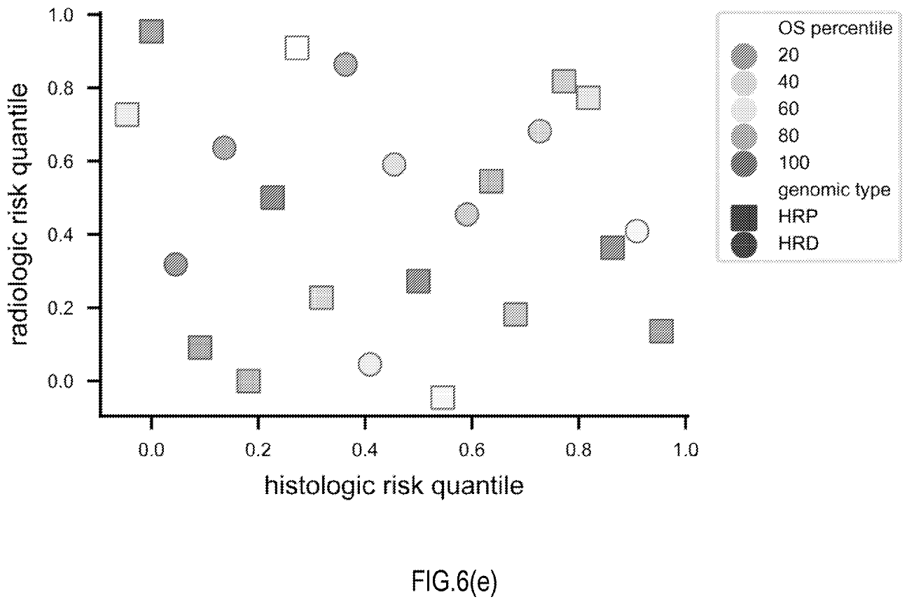

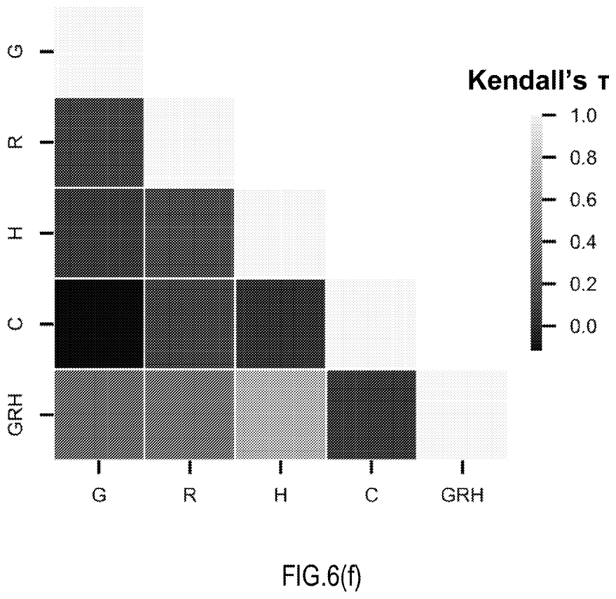

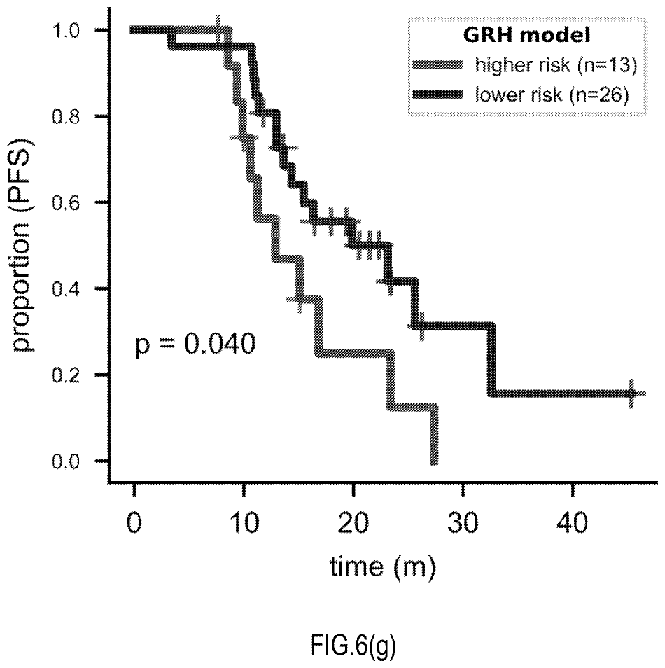

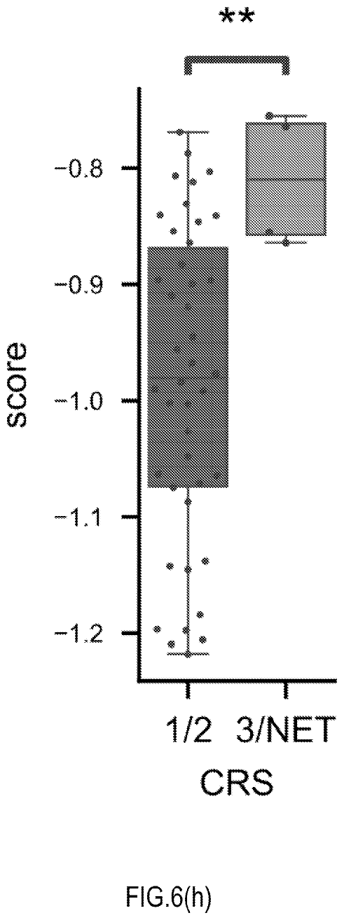

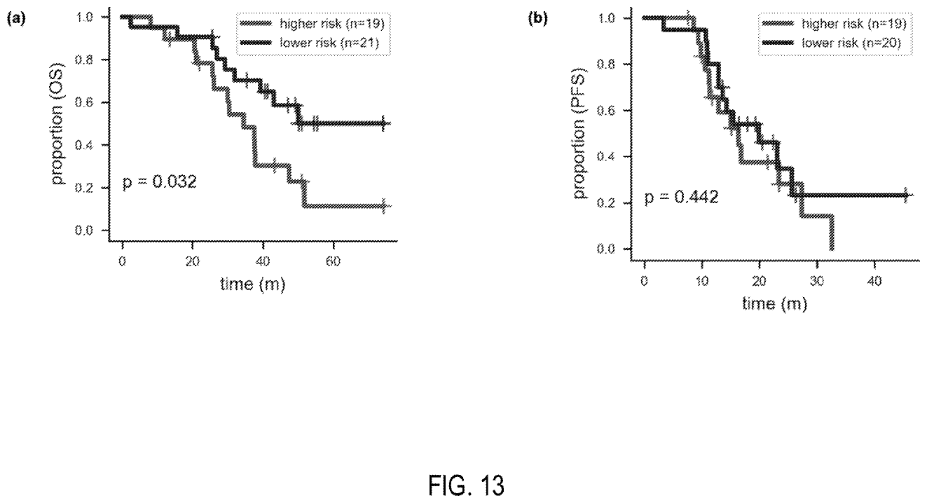

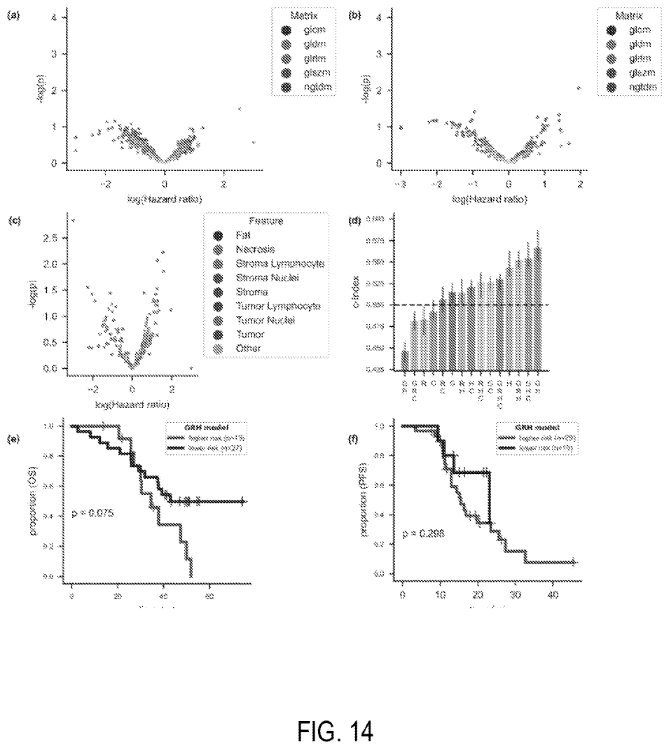

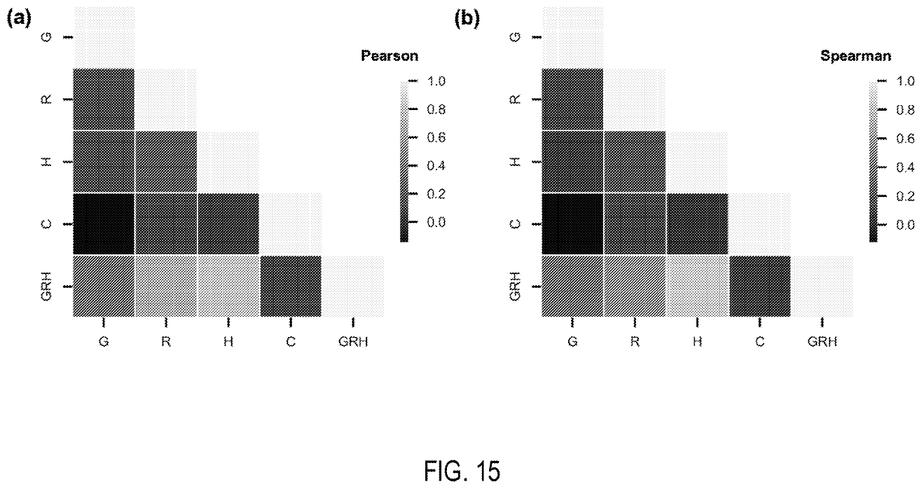

The following were tested: prognostic significance of patient age, pathologic stage, residual disease status after debulking surgery, NACT-IDS versus PDS treatment paradigm, receipt of PARP inhibitors in the first two years after diagnosis, and the presence or absence of adnexal lesions (Supplementary Table 6), ultimately training a model on residual disease status and PARP inhibitor administration. This model stratified the test set with c=0.51 [95% C.I. 0.493-0.528]. A late-fusion approach were then implemented to integrate histopathologic, radiomic, genomic, and clinical data into multimodal models (FIG. 1e). Specifically, each patient's log partial hazard is predicted using the Cox model trained using the respective modality, then trained a final Cox model to integrate them (Methods). In the test set, the model combining both imaging modalities (radiomic-histopathologic, RH model) significantly outperformed the HRD status-based model, clinical model, and individual imaging models, with a test concordance index of 0.62 [95% C.I. 0.604-0.638] (FIG. 6a). The model with genomic, radiomic, and histopathologic (GRH) modalities performed comparably, with a test concordance index of 0.61 [95% C.I. 0.594-0.625]. The histopathologic submodel score remained significant upon addition of HRD status (FIG. 6b). The high- and low-risk groups established by the GRH model were significantly different by log-rank test in the training set (median survival of 34 and 50 months, respectively (p=0.026; FIG. 6c). In the test set, the GRH risk groups also showed significantly different OS, with median survival of 30 months for the high-risk group and 50 months for the low-risk group (p=0.023; FIG. 6d). At 36 months, 68% and 34% survived for low- and high-risk groups, respectively, in the test set. The separation of the RH model's risk groups was inferior (FIG. 13). Notably, analysis of only training cases with full information (n=114) resulted in poor performance (FIG. 14), reinforcing the ability of late fusion models to learn in the setting of missing data. No robust association was found between modalities to enable interpolation of missing values (FIG. 15).

The c-indices for individual imaging modalities were similar, but identified distinct patient subgroups with good prognosis (FIG. 6e). This is consistent with radiologic and histologic features containing complementary information content, whereby some patients with good outcomes were identified as high risk by the radiomic sub-model but correctly assigned a lower risk score by the histopathologic sub-model, and vice versa. Patients with HRD and HRP disease were distributed relatively evenly, agnostic to unimodal imaging risk scores.

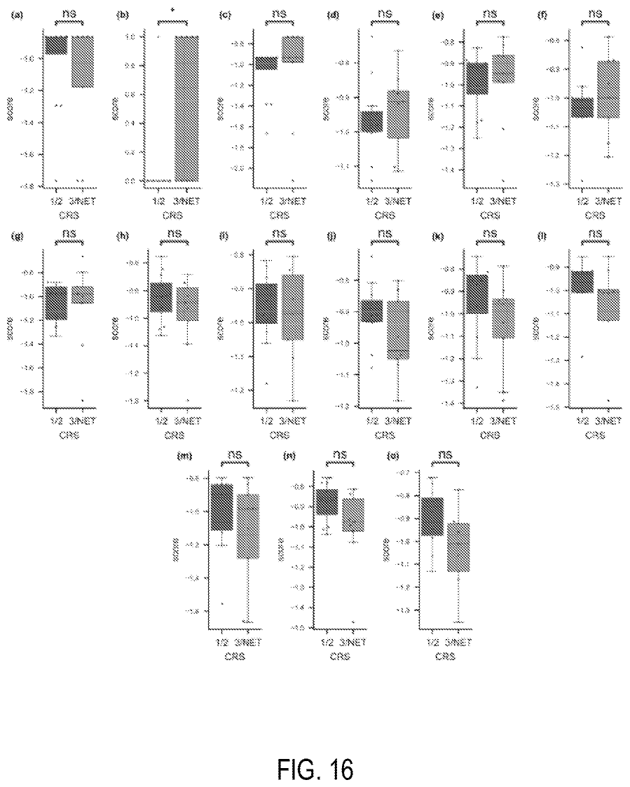

Corroborating this, absolute Kendall rank correlation coefficient values were low between individual modalities (<0.14) (FIG. 6f), demonstrating that the radiomic and histopathologic models ordered patients differently as compared to the genomic model and to one another. The same two risk groups identified by the model in the test set also showed significantly different progression-free survival (p=0.040; FIG. 6g). Finally, as an orthogonal validation, the inferred risk of all models except the G and GH models associated with pathologic chemotherapy response score (CRS) in the training set, including the GHR model (FIG. 6h). The test set had only 21 patients with known CRS, and only HRD status exhibited statistically significantly different distributions of CRS by the Mann-Whitney U test in the test set (FIG. 16).

### Discussion

Machine learning in cancer prognostics is a growing field with great potential, but the contribution of common diagnostic modalities to multimodal risk stratification remains poorly understood. Here, it is shown that integrating multi-scale clinical imaging and genomic data increases predictive capacity. These results, in addition to the low correlation between risk scores derived from individual modalities, support the hypothesis that clinical imaging contains complementary prognostic information that is independent of clinico-genomic information. Histopathologic and radiologic imaging characterize the tumor architecture at microscopic and mesoscopic scales, respectively. Therefore, it stands to reason that these data channels complement one another and HRD status, which is derived from spatially-agnostic sequencing. The full GHRC model did not perform as well as the RH and GRH models, suggesting that multimodality is not a universal guarantee of improved performance. In this case, the most likely reason is that the clinical model (based on history of PARP inhibitor administration and residual disease status after debulking surgery) does not stratify the test cohort, likely due to its small size. Furthermore, the TCGA cohort did not have these informative clinical variables available. The late fusion architecture benefits from few parameters to fit—which reduces overfitting—and the ability to learn from partial information cases, but it cannot gate information from noisy modalities. With larger datasets enabling more parameter fitting without overfitting, mechanisms such as attention can be explored to adaptively adjust unimodal contributions.

In addition to multimodal integration, two unimodal models are presented to stratify late-stage HGSOC patients using routine clinical imaging, validated these models on a test set, and studied the relative contributions of each modality to risk-stratifying HGSOC patients. For radiologic imaging, it is discovered that omental autocorrelation computed from the gray level co-occurrence matrix derived from the HLL Coif wavelet-filtered image was a prognostic feature. This Imaging Biomarker Standardization Initiative-defined feature has been found to be strongly or very strongly reproducible in multiple studies. It describes the coarseness of the lesion texture and also depends on tissue density. Seven of the other nine omental features with significant log(HR) values were explicitly designed to measure high-density zones, and these features did not exhibit log(HR) values significantly different from zero on multivariable regression with the autocorrelation. Hence, the most parsimonious explanation is that higher-density—rather than coarser—omental implants are an adverse prognostic factor, which could be due to more solid tumors with reduced cystic or fatty components. Omental textures captured by autocorrelation may also reflect differing intratumoral heterogeneity.

Other HGSOC radiomic models have not explored the prognostic information captured within omental implants, relying instead on more demanding segmentations of adnexal lesions or the entire tumor burden. Interestingly, it was found that none of the radiomic features derived from adnexal masses had log(HR) values significantly different from zero after correction for multiple hypothesis testing, which is possibly due to the late stage of this cohort: the omentum is the most common site of metastasis in HGSOC and may drive further peritoneal seeding. An omental model is advantageous over an adnexal model because omental implants are ubiquitous in advanced stage disease, even in patients with primary peritoneal high-grade serous cancer that lack adnexal mass(es). Furthermore, an omental implant can be readily segmented even by less experienced observers, whereas adnexal masses can be challenging to distinguish from adjacent loculated ascites, serosal and pouch of Douglas implants, and adjacent anatomic structures such as the uterus, especially in the presence of leiomyomas. An omental model is also more practical than a radiomic model based on the whole tumor burden; routine segmentation of the whole tumor volume is impractical in daily practice using current tools due to prohibitively high demand for time and expertise.

For histopathologic imaging, an H&E WSI-based model is developed to stratify HGSOC patients. Although none of the features exhibited log(HR) values significantly different from zero after correction for multiple hypothesis testing, the presence of 20 features highly related to mean tumor nuclear size (e.g., 60th percentile of tumor nuclear size, 50th percentile of tumor nuclear diameter) with similar hazard ratios in the 24 features with uncorrected significant p-values for univariate log(HR) values supports the prognostic relevance of tumor nuclear size. This is further supported by the good stratification of the test set. The larger nuclear size may be associated with events such as whole-genome doubling or cellular fusion and warrants direct study of matched genomes and histopathologic sections. The major axis length of stroma is difficult to interpret for a two-dimensional slice of tissue but may reflect distinct patterns of disease infiltration into surrounding stroma. The trained weights are included for the HGSOC model, and the source code is included for extension to other cancer types.

This lack of usable large datasets is one of the main challenges for multimodal machine learning in oncology. Data is created from the 296 MSKCC HGSOC patients available to enable work toward improving upon the models presented here. These results demonstrate the benefit of learning from cases with only partial information in multimodal studies: the smaller, full-information sub-cohort yielded a significantly less generalizable risk stratification model. The dataset also offers the advantage of comprising H&E images and CE-CT scans originally acquired at multiple institutions: this improves confidence in the generalizability of the results. Furthermore, data generated during the standard of care was intentionally mind. Using these data instead of specialty research data drastically reduces adoption costs in the clinical workflow for resultant models, but the data were not collected specifically with computational modeling in mind. For example, some patients with only germline sequencing of HRD-DDR genes were included, a clinically relevant but biologically imperfect measure of HRD status: each risk group is enriched for—but not exclusively composed of—the genomic subtype of interest. It is expected that clinical whole-genome sequencing will enable more robust genomic analyses.

The improved risk stratification models developed herein show the promise of extracting and integrating quantitative clinical imaging features toward aiding gynecologic oncologists in selecting primary treatment, planning surveillance frequency, making decisions about maintenance therapy, and counseling patients about clinical trials of investigative agents. The statistical robustness and clinical relevance of the risk groups by both PFS and OS in the test set substantiate the utility of this multimodal machine learning approach, establishing proof of principle. Next steps include scaled and inter-institutional retrospective cohort assembly for further model training and refinement before prospective validation of clinical benefit in randomized controlled trials.

In summary, a multimodal dataset of HGSOC patients is assembled and this dataset is used to develop and integrate radiologic, histopathologic, and clinico-genomic models to risk-stratify patients. It is discovered that the autocorrelation of omental implants on CE-CT and average tumor nuclear size on H&E are prognostic factors, that these modalities are demonstrably orthogonal, and that their computational integration improves stratification beyond previously known clinico-genomic factors in a test set. These results motivate further large-scale studies driven by multimodal machine learning to stratify cancer patients, both in HGSOC and other cancer subtypes.

### Methods

This study complies with all relevant ethical regulations, and its protocols were approved an institutional review board. Informed consent was waived for this retrospective study, and participants were not compensated.

### Cohort Curation

Patients were eligible for this retrospective study if they had biopsy-proven newly diagnosed high-grade serous ovarian cancer and at least one of (A) pre-treatment whole-slide images of H&E depicting high-grade serous carcinoma or (B) pre-treatment contrast-enhanced abdominal/pelvic computed tomography (CE-CT). Most of the MSKCC cohort was sourced from a retrospective clinical database of patients who underwent diagnostic workup and NACT-IDS at the institution. This database also contained information on the residual disease status after debulking surgery, pathologic stage, administration of neoadjuvant chemotherapy, and patient age at diagnosis from the electronic medical record. To expand the cohort, the institutional data warehouse is searched for patients with sequencing and available pretreatment CT studies or H&E images. In addition to this retrospective curation, 36 patients were also included from the prospective project. Pathologic stage was unavailable for 14 patients, and instead the clinical stage were recorded as in the Institutional Database for these patients. Also, the race were collected for all patients from the institutional data warehouse. Overall and progression-free survival were calculated using the date of CT as a start date, when available, or the date of pathologic diagnosis otherwise.

To collect the H&E imaging, the EHR is reviewed to find associated pathology cases with peritoneal lesions (primarily omental), and expert pathologists reviewed the slides to select high-quality specimens for digitization. The institutional data repository was also reviewed for scanned slides associated with the diagnostic biopsy and included those containing tumors. All H&E imaging was pretreatment.

Subsequently, the associated CE-CT scans are reviewed for the following the inclusion criteria: 1) intravenous contrast-enhanced images acquired in the portal venous phase, 2) absence of streak artifacts or motion-related image blur obscuring lesion(s) of interest, and 3) adequate signal to noise ratio (Supplementary Table 7). All CE-CT imaging was pretreatment. All CT scans were available in the digital imaging and communications in medicine (DICOM) format through an institutional picture archiving and communication system (PACS, Centricity, GE Medical Systems v. 7.0).

### TCGA Cohort Selection

From the TCGA-OV project, patients were searched with clinical data annotated in the TCGA Clinical Data Resource, pathologic grade, and at least one of a diagnostic FFPE H&E WSIs or abdominal/pelvic CE-CT scan in the TCIA. All clinical and demographic information were extracted from the TCGA CDR. Only diagnostic WSIs of formalin-fixed, paraffin-embedded H&E-stained specimens from the TCGA-OV project were included. All H&E imaging was pre-treatment.

All CT scans met the following the inclusion criteria: 1) intravenous contrast-enhanced images acquired in the portal venous phase, 2) absence of streak artifacts or motion-related image blur obscuring lesion(s) of interest, and 3) adequate signal to noise ratio (Supplementary Table 7). All CE-CT imaging was pretreatment.

Inferring HRD status. In the MSKCC cohort, MSK-IMPACT clinical sequencing is used, when available, to infer HRD status. Variant calling for these genes and copy number analysis of CCNE1 was performed using a clinical pipeline. For patients with appropriate consent for further genomic re-analysis, COSMIC SBS3 activity is also inferred using SigMA (for cases with at least five mutations across all 505 genes) and searched for large-scale state transitions using another pipeline. OncoKB and Hotspot annotations were also used for variant significance in genes involved in HRD-DDR to assign patients to the HRD subtype. Patients with high-confidence dominant signature 3 or at least one significant variant or deep deletion in the HRD-DDR genes were assigned to the HRD subtype, except when there was evidence that patients belonged to the foldback inversion- or tandem duplicator-enriched subgroups (via CCNE1 amplification or CDK12 SNVs, specifically): These patients with conflicting evidence were assigned to the ambiguous subtype and excluded from analysis. Low-confidence signature 3 results were not used for HRD status definition. Incorporating LST thresholding to define HRD status was found to diminish the separation of the HRD and HRP-defined groups in the training set (FIG. 8a,f), and thus it was not used in the final HRD status definition. Patients with available results from clinical HRD-DDR panels or BRCA1/2 send out panels were assigned HRP unless there were variants of known significance (as determined by the test provider) in at least one reported gene.

In the TCGA cohort, CNA and SNV data were downloaded from the TCGA-OV project on cBioPortal for the same set of genes implicated in HRD-DDR, CDK12, and CCNE1, again filtering to variants deemed significant by OncoKB. Using these criteria, patients with at least one SNV or deep deletion in HRD-DDR genes were assigned the HRD subtype. Patients without aberrations in these HRD-DDR-associated genes were assigned the HRP subtype. Patients with an SNV in CDK12 or amplification in CCNE1 and also with an SNV in at least one of the HRD-DDR genes were assigned the ambiguous subtype and excluded from analysis. Patients without available SNV and CNA data in cBioPortal were assigned to the ambiguous subtype and excluded. COSMIC SBS3 frequencies were downloaded from Synapse, which is clearly bimodal (FIG. 9c), and patients with SBS3 frequency greater than 15% and without conflicting evidence of HRP were assigned to the HRD subtype.

### Adnexal and Omental Lesions Segmentation

Three fellowship-trained radiologists manually segmented ovarian lesions and representative omental implants on each pretreatment CE-CT scan for all patients (MSKCC and TCGA-OV/TCIA). Using the Insight Segmentation and Registration Toolkit-SNAP version 3.8.0 software, each radiologist traced the outer contour of ovarian and omental lesions on every tumor-containing axial section. All questions that arose during segmentation were resolved via joint review and consensus.

### Train-Test Split

40 training cases were sampled randomly before analysis from the patients with available H&E WSI, unambiguous HRD status, known stage, and omental lesion on CE-CT. This strategy is used to enable fair comparisons across unimodal and multimodal models, preventing spurious differences in test concordance indices due to patient exclusion for some models but not for others. Both TCGA-OV and MSKCC cases are included in the training and test sets: this is because only 4 TCGA cases had complete information from all modalities and thus could not support a fully external test set.

### Radiologic Feature Extraction

All DICOM series are converted to volumetric images in Hounsfield Units and applied an abdominal window (level 50, width 400). Using PyRadiomics, images were resampled to isotropic 1 mm³ voxels using the Simple ITK B-spline interpolator and binned images with bin size of 25 HU. Features in 3D were extracted from Coif wavelet-transformed images. Features were extracted from the gray level size zone, neighboring gray tone difference, gray level run length, gray level dependence, and gray level co-occurrence matrices, yielding a representation of each study's representative omental lesion(s) or individual adnexal lesion(s).

### Histopathologic Annotation

Two expert pathologists partially annotated 60 H&E WSIs using the Slide Viewer. The approach was to label example regions of necrosis, lymphocyte-rich tumor, lymphocyte-poor tumor, lymphocyte-rich stroma, lymphocyte-poor stroma, veins, arteries, and fat with reasonable but imperfect accuracy. These annotations are exported as bitmaps and converted them to GeoJSON objects. Lymphocyte-rich/poor tumor labels and lymphocyte-rich/poor stroma labels are amalgamated for training and omitted vessels from the training data for the models presented herein. Next, these annotations are used to generate tissue-type tiles.

### Training the Histopathologic Tissue Type Classifier

Tiles measuring 64 μm×64 μm (128×128 pixels) with 50% overlap are generated, using the above annotations to delineate regions to be tiled. No other tile sizes were explored; this size was chosen because it offered good resolution while still depicting multiple cells in each tile. Putative tile squares within an annotation but with <20% foreground as assessed by Otsu's method were not tiled. Macenko stain normalization was used. A ResNet-18 model (pretrained on ImageNet) are trained for 30 epochs with a learning rate of 5e-4, 1e-4 L2 regularization, and the Adam optimizer. The objective function was class-balanced cross entropy, and mini batches of 96 tiles are used on a single NVIDIA Tesla V100 GPU. Four-fold, slide-wise cross-validation are used for model evaluation and hyperparameter tuning. The number of epochs are selected to train the final model using the epoch with the highest lower 95% C.I. bound estimated using the mean and standard deviation of the cross-validation F1 scores. The model is trained on tiles from all 60 slides for 21 epochs.

### Histopathologic Feature Extraction and Selection

The WSIs associated with the patients in this cohort are tiled without overlap, performing inference using mini batches of 800 across four NVIDIA Tesla V100 GPUs. Macenko stain normalization is used for all slides because staining intensity differences from the predominantly MSKCC-based training cohort confounded inference. Tile predictions are assembled into downscaled bitmaps, which were then used to calculate tissue-type features in an approach. The region properties from scikit-image are included for both the largest connected component and the entirety of each tissue type. Features such as the area ratio of one tissue type to another and the entropy of tumor and stroma are also calculated. Using the StarDist method for QuPath, individual nuclei are segmented and characterized, using nuclei with a detection probability greater than 0.5. A lymphocyte classifier trained iteratively using manual annotations is used to distinguish lymphocytes from other cells. A tissue parent type is assigned to each nucleus using the inferred tissue type maps and calculated aggregative statistics by tissue type and cell type of the QuPath-extracted nuclear morphologic and staining features, such as variance in eosin staining or circularity. Together, these cell type features and tissue type features based on tumor, stroma, and necrosis constituted the histopathologic embedding for each slide.

### Clinical Data Encoding

Residual disease status after debulking surgery was encoded as a binary variable, where patients with ≤1 cm residual disease (including complete gross resection) were assigned a value of 1, and patients with >1 cm residual disease were assigned a value of 0. The presence of adnexal lesions on CE-CT was also included as a binary variable. Age at diagnosis was modeled as a continuous variable scaled by the training set range. Tumor stage was encoded as one-hot categorical variables for I, II, III, IV, and Unknown. Similarly, the primary treatment approach was encoded as a one-hot categorical variable with values NACT-IDS, PDS, and Unknown.

### Feature Selection

The same strategy was used to select radiomic, histopathologic, and clinical features. For each feature, a univariate Cox Proportional Hazards model is fit to the full training set using the Python Lifelines package without regularization, and the univariate coefficient and significance confidence are plotted. For features whose model failed to converge, fitting is re-attempted with L2 regularization C=0.2, and any model still failing to converge was assigned a log Hazard Ratio of 0 and p-value of 1. For histopathology, relative specimen size is controlled for by including it in each Cox model. Next, features with scaled interquartile range below 0.1 are removed. Subsequently, for radiomics, which is the largest feature space, the Benjamini-Hochberg method is used to correct for multiple hypothesis testing. Taking the ordered list of features significant with 95% confidence, Algorithm 1 is applied to select features, yielding modality signatures with low multicollinearity.

```
Algorithm 1 Multivariable model selection procedure
Input: A list of unique candidate features ordered by p-value fi where i ∈
[1,k].
Output: A list of features significant with confidence α on multivariable
regression gj where j ∈ [1,l] and l ≤ k.
Require: k ≥ 1 i ← 1 j ← 1
  while i ≤ k do
    gj ← fi
    p ←significance(g) [symbol]   significance assessed by Cox regression
    if pj < α then j ← j + 1
    end if
    i ← i + 1
end while
```

The only modification to this procedure occurred for the ablation experiment to test the importance of learning from the partial information cases: a threshold of 0.31 is used for clinical features since none were significant with p<0.05, and multiple hypothesis testing is not corrected for in the omental radiomic features during the ablation experiment since none would be significant by this metric.

### Survival Modeling

Linear Cox Proportional Hazards models are used with L2 regularization (C=0.5) and no L1 regularization for all multimodal and unimodal models. No sub-model was fit for the genomic modality: patients assigned to the HRP subtype were designated high risk (risk score=1.0), and patients assigned to the HRD subtype were designated low risk (risk score=0.0). No interaction terms were used.

Kaplan Meier analysis is used to determine whether each model stratified patients into clinically significant groups. To delineate group membership, percentile thresholds is tested in {0.33, 0.34, . . . , 0.64, 0.65 0.66}, choosing the value that maximized significance of the separation in the training set by the log-rank test. This was performed individually for OS and PFS, where relevant. P-values for concordance indices were calculated using 1000-fold permutation tests. 95% confidence intervals for c-indices were calculated using 100-fold leave-one-out bootstrapping. All p-values for Kaplan-Meier analysis were calculated by the multivariate log-rank test. P-values for covariate significance in Cox Proportional Hazards models are reported for models fit with C=0.5. Fraction surviving was estimated using linear interpolation.

### Multimodal Integration

A late fusion approach is chosen to increase unimodal sample sizes available for parameter estimation. Parameters for unimodal sub-models were estimated using all available unimodal data (e.g., radiomic parameters were estimated across the 251 training CT cases with omental lesions, and histopathologic parameters were estimated across the 243 training H&E cases), where each sub-model inferred a partial hazard for each patient. The negative partial hazard was used to enable compatibility with the concordance index as implemented in the lifelines Python package. For the second-stage late fusion model, parameters are estimated for a multivariate Cox model integrating the negative log partial hazards inferred by each modality using only the intersection set of patients.

### Statistics and Reproducibility

No statistical method was used to predetermine sample size. Data were excluded from the analyses only for the reasons detailed above and prior to any machine learning modeling. The training and test sets were chosen at random from the patients with all four data modalities available. The investigators were not blinded to allocation during outcome assessment. Data distributions were not assumed to be normal for any tests. The hazards were assumed to be proportional for survival modeling, but this was not formally tested.

### APPENDIX

SUPPLEMENTARY TABLE 1

| | Training (N = 404) | | Test (N = 40) | |
|---|---|---|---|---|
| Median age at diagnosis | 63 years [IQR 55-71] | | 66 years [IQR 59-70] | |
| Stage | | | | |
| I | 8 | (2%) | 0 | (0%) |
| II | 10 | (2%) | 0 | (0%) |
| III | 225 | (56%) | 9 | (23%) |
| IV | 160 | (40%) | 31 | (78%) |
| Unknown | 1 | (0%) | 0 | (0%) |
| Treatment | | | | |
| NACT-IDS | 175 | (43%) | 31 | (78%) |
| PDS | 82 | (20%) | 8 | (20%) |
| Unknown | 147 | (36%) | 1 | (3%) |
| Debulking outcome | | | | |
| ≤1 cm residual disease | 148 | (37%) | 28 | (70%) |
| >1 cm residual disease | 48 | (12%) | 11 | (28%) |
| Unknown | 208 | (51%) | 1 | (3%) |
| Adnexal lesion | | | | |
| on CE-CT | | | | |
| Present | 243 | (60%) | 29 | (73%) |
| Absent | 59 | (15%) | 11 | (28%) |
| Unknown | 102 | (25%) | 0 | (0%) |
| Race | | | | |
| Asian | 25 | (6%) | 5 | (13%) |
| Black | 29 | (7%) | 1 | (3%) |
| White | 329 | (81%) | 33 | (83%) |
| Other | 8 | (2%) | 0 | (0%) |
| Unknown | 13 | (3%) | 1 | (3%) |
| Received PARP in | | | | |
| first 2 yrs | | | | |
| Received | 51 | (13%) | 10 | (25%) |
| Did not receive | 206 | (51%) | 29 | (73%) |
| Unknown | 147 | (36%) | 1 | (3%) |

SUPPLEMENTARY TABLE 2

| | Training (N = 404) | Test (N = 40) |
|---|---|---|
| Overall survival | | |
| Available | 404 (100%) | 40 (100%) |
| Duration | 38.7 months [IQR 25-55] | 37.6 months [IQR 26-49] |
| Censored | 132 (33%) | 17 (43%) |
| Progression- | | |
| free survival | | |
| Available | 383 (95%) | 39 (98%) |
| Duration | 15.5 months [IQR 11-23] | 15.1 months [IQR 11-21] |
| Censored | 108 (28%) | 14 (36%) |

SUPPLEMENTARY TABLE 3

| | |
|---|---|
| Training (N = 298) | Test (N = 40) |

| | | | | |
|---|---|---|---|---|
| CT Vendor | | | | |
| GE | 213 | (71%) | 30 | (75%) |
| Siemens | 66 | (22%) | 5 | (13%) |
| Philips | 13 | (4%) | 3 | (8%) |
| Toshiba | 6 | (2%) | 1 | (3%) |
| Imatron | 0 | (0%) | 1 | (3%) |
| Acquisition site | | | | |
| External | 223 | (75%) | 24 | (60%) |
| MSKCC | 75 | (25%) | 16 | (40%) |
| Segmenting radiologist | | | | |
| YL | 175 | (59%) | 18 | (45%) |
| EA | 65 | (22%) | 12 | (30%) |
| IN | 58 | (19%) | 10 | (25%) |

| | | |
|---|---|---|
| Slice thickness (mm) | 5.0 [5.0- | 5.0 [5.0- |
| | 5.0] (2.5-7.5) | 5.0] (2.5-5.0) |
| Peak kilovoltage | 120 [120- | 120 [120- |
| | 120] (90-140) | 120] (100-130) |
| Tube current (mA) | 278 [219- | 348 [219- |
| | 380] (80-699) | 380] (118-748) |

SUPPLEMENTARY TABLE 4

| feat | p | stat | p_corrected |
|---|---|---|---|
| wavelet-HLL_glcm_Autocorrelation | 8.61E−05 | 1.679960254 | 0.009386627 |
| wavelet-HLL_gldm_HighGrayLevelEmphasis | 9.00E−05 | 1.671695797 | 0.009386627 |
| wavelet-HLL_glrlm_HighGrayLevelRunEmphasis | 9.10E−05 | 1.661559471 | 0.009386627 |
| wavelet-HLL_gldm_LargeDependenceHighGrayLevelEmphasis | 0.000110485 | 1.721512331 | 0.009386627 |
| wavelet-HLL_glszm_HighGrayLevelZoneEmphasis | 0.000112994 | 1.687129942 | 0.009386627 |
| wavelet-HLL_glcm_SumAverage | 0.000148725 | 1.626796351 | 0.009386627 |
| wavelet-HLL_glcm_JointAverage | 0.000148725 | 1.626796351 | 0.009386627 |
| wavelet-HLL_glrlm_ShortRunHighGrayLevelEmphasis | 0.000169128 | 1.603570336 | 0.009386627 |
| wavelet-HLL_glrlm_LongRunHighGrayLevelEmphasis | 0.000256773 | 1.683558961 | 0.012667476 |
| wavelet-HLL_glcm_Idn | 0.001318066 | 1.374884033 | 0.057752072 |
| wavelet-HLL_glcm_Idmn | 0.001482739 | 1.329095017 | 0.057752072 |
| wavelet-HLL_glszm_SmallAreaHighGrayLevelEmphasis | 0.001560867 | 1.199763279 | 0.057752072 |
| wavelet-HLL_ngtdm_Complexity | 0.003100837 | 1.153518813 | 0.105905502 |
| wavelet-HLL_glrlm_LowGrayLevelRunEmphasis | 0.004210684 | −2.039724711 | 0.125282032 |
| wavelet-HLL_gldm_SmallDependenceHighGrayLevelEmphasis | 0.004232501 | 1.184562094 | 0.125282032 |
| wavelet-HLL_ngtdm_Contrast | 0.006166151 | −1.271601861 | 0.171110692 |
| wavelet-HLL_glrlm_ShortRunLowGrayLevelEmphasis | 0.011061711 | −1.637074448 | 0.288905858 |
| wavelet-HLL_glszm_LowGrayLevelZoneEmphasis | 0.015601498 | −1.826227832 | 0.384836962 |
| wavelet-HHH_glszm_GrayLevelNonUniformity | 0.020387278 | 1.086720405 | 0.4764185 |
| wavelet-HLL_gldm_LargeDependenceLowGrayLevelEmphasis | 0.022643297 | −1.607266735 | 0.502681195 |
| wavelet-HLH_glszm_ZoneEntropy | 0.030829015 | 1.211542547 | 0.627516998 |
| wavelet-HLL_glrlm_LongRunLowGrayLevelEmphasis | 0.036659477 | −1.264303146 | 0.627516998 |
| wavelet-HHH_glszm_SizeZoneNonUniformity_Normalized | 0.039144001 | −0.589140982 | 0.627516998 |
| wavelet-HHH_glszm_ZoneEntropy | 0.039210482 | 0.654005489 | 0.627516998 |
| wavelet-HHH_glcm_Imc2 | 0.039213311 | −1.046599168 | 0.627516998 |
| wavelet-HHH_glcm_MaximumProbability | 0.039289584 | −0.718539255 | 0.627516998 |
| wavelet-LLH_glrlm_HighGrayLevelRunEmphasis | 0.041058672 | 0.869936723 | 0.627516998 |
| wavelet-LLH_glrlm_LongRunHighGrayLevelEmphasis | 0.043024389 | 0.836549447 | 0.627516998 |
| wavelet-LLH_gldm_HighGrayLevelEmphasis | 0.044272506 | 0.861399376 | 0.627516998 |
| wavelet-LLH_glcm_Autocorrelation | 0.044982933 | 0.860002716 | 0.627516998 |
| wavelet-HHH_glcm_Imc1 | 0.046187366 | 0.794199348 | 0.627516998 |
| wavelet-LLH_glrlm_ShortRunHighGrayLevelEmphasis | 0.047009389 | 0.886220124 | 0.627516998 |
| wavelet-LLH_glcm_JointAverage | 0.048053103 | 0.737160058 | 0.627516998 |
| wavelet-LLH_glcm_SumAverage | 0.048053103 | 0.737160058 | 0.627516998 |
| wavelet-HHH_glcm_MCC | 0.052069583 | −0.683613678 | 0.642056171 |
| wavelet-HLL_gldm_SmallDependenceLowGrayLevelEmphasis | 0.054407672 | −1.040118994 | 0.642056171 |
| wavelet-HHH_glcm_DifferenceEntropy | 0.055933285 | 0.728658603 | 0.642056171 |
| wavelet-LLH_gldm_LargeDependenceHighGrayLevelEmphasis | 0.056293748 | 0.807640346 | 0.642056171 |
| wavelet-HLL_glcm_MCC | 0.056396826 | 0.901155284 | 0.642056171 |
| wavelet-LLH_gldm_LargeDependenceLowGrayLevelEmphasis | 0.061433325 | −0.526276039 | 0.681909911 |
| wavelet-HLL_glszm_ZoneEntropy | 0.064166337 | 0.945630694 | 0.694874484 |
| wavelet-LLH_glszm_GrayLevelNonUniformity | 0.068680148 | 0.80561964 | 0.714680962 |
| wavelet-LLL_glszm_ZoneEntropy | 0.069349266 | 0.79104129 | 0.714680962 |
| wavelet-LLH_gldm_LowGrayLevelEmphasis | 0.07082424 | −0.467273968 | 0.714680962 |
| wavelet-LLH_glszm_HighGrayLevelZoneEmphasis | 0.074261461 | 0.792347555 | 0.720804035 |
| wavelet-HLL_glszm_SmallAreaLowGrayLevelEmphasis | 0.074677896 | −0.992883949 | 0.720804035 |
| wavelet-LLH_glrlm_LongRunLowGrayLevelEmphasis | 0.079182468 | −0.619575519 | 0.748021614 |
| wavelet-LLH_glrlm_LowGrayLevelRunEmphasis | 0.084469022 | −0.438852194 | 0.781338453 |
| wavelet-LHL_glcm_Imc2 | 0.088067319 | −0.960529362 | 0.797997749 |
| wavelet-LLH_gldm_SmallDependenceLowGrayLevelEmphasis | 0.091059415 | −0.626970891 | 0.808607604 |
| wavelet-HLL_glszm_SmallAreaEmphasis | 0.100928836 | 0.828358623 | 0.878674576 |
| wavelet-HHL_glcm_Imc1 | 0.104943919 | 0.68426125 | 0.896059618 |
| wavelet-HLL_glszm_GrayLevelVariance | 0.109273382 | 0.919680556 | 0.897626166 |
| wavelet-HHH_glcm_Id | 0.115697603 | 1.429180802 | 0.897626166 |
| wavelet-LLH_glcm_Idmn | 0.124219517 | 0.519626387 | 0.897626166 |
| wavelet-HLL_glszm_GrayLevelNonUniformityNormalized | 0.124737069 | −0.637806414 | 0.897626166 |
| wavelet-LLH_glszm_SizeZoneNonUniformity | 0.132957316 | 0.677810956 | 0.897626166 |
| wavelet-LLL_glszm_GrayLevelNonUniformity | 0.136431509 | 0.655638427 | 0.897626166 |
| wavelet-HLL_glrlm_RunEntropy | 0.137959367 | 0.837928585 | 0.897626166 |
| wavelet-HHL_glcm_Imc2 | 0.139477678 | −0.746816789 | 0.897626166 |
| wavelet-LLH_glrlm_ShortRunLowGrayLevelEmphasis | 0.141097451 | −0.459135365 | 0.897626166 |
| wavelet-LLH_glszm_SmallAreaHighGrayLevelEmphasis | 0.146601969 | 0.63962502 | 0.897626166 |
| wavelet-LLL_glszm_LowGrayLevelZoneEmphasis | 0.161742006 | −0.77573616 | 0.897626166 |
| wavelet-LLH_ngtdm_Contrast | 0.166050254 | −0.416106814 | 0.897626166 |
| wavelet-HHH_glcm_JointEnergy | 0.166412014 | −1.10164689 | 0.897626166 |
| wavelet-LHH_glcm_Imc2 | 0.174503199 | −0.702144504 | 0.897626166 |
| wavelet-HHH_glcm_Idmn | 0.176961858 | 0.610968027 | 0.897626166 |
| wavelet-LHH_glszm_SmallAreaLowGrayLevelEmphasis | 0.177944261 | 0.53267898 | 0.897626166 |
| wavelet-HHH_ngtdm_Contrast | 0.182165569 | −0.566861775 | 0.897626166 |
| wavelet-LHH_glszm_ZoneEntropy | 0.182676215 | 0.743405161 | 0.897626166 |
| wavelet-HHH_glcm_Correlation | 0.184859892 | 0.592816925 | 0.897626166 |
| wavelet-LHH_glcm_MaximumProbability | 0.194744635 | −0.874366468 | 0.897626166 |
| wavelet-LHH_glcm_Imc1 | 0.196879175 | 0.712578228 | 0.897626166 |
| wavelet-HHL_glcm_Correlation | 0.204287582 | 0.607330154 | 0.897626166 |
| wavelet-LHH_glcm_MCC | 0.20627069 | −0.624449188 | 0.897626166 |
| wavelet-LLL_glrlm_RunLengthNonUniformity | 0.206824571 | 0.528899722 | 0.897626166 |
| wavelet-LLL_glrlm_LowGrayLevelRunEmphasis | 0.209340863 | −0.689785452 | 0.897626166 |
| wavelet-HHL_gldm_Dependence_Variance | 0.21116865 | −0.434364713 | 0.897626166 |
| wavelet-HHH_glcm_Inverse_Variance | 0.211321892 | −0.578555986 | 0.897626166 |
| wavelet-HHL_gldm_DependenceNonUniformityNormalized | 0.211344077 | 0.495409186 | 0.897626166 |
| wavelet-LLL_glszm_GrayLevelNonUniformityNormalized | 0.216679031 | −0.666014605 | 0.897626166 |
| wavelet-HHH_glszm_SmallAreaEmphasis | 0.218450537 | 0.333109022 | 0.897626166 |
| wavelet-LLL_gldm_LowGrayLevelEmphasis | 0.224071725 | −0.697777767 | 0.897626166 |
| wavelet-LLH_glszm_LowGrayLevelZoneEmphasis | 0.227164087 | −0.560747133 | 0.897626166 |
| wavelet-LHH_glszm_GrayLevelNonUniformity | 0.230869062 | 0.466010864 | 0.897626166 |
| wavelet-LLL_glrlm_ShortRunLowGrayLevelEmphasis | 0.231566324 | −0.586825733 | 0.897626166 |
| wavelet-LHH_glszm_SizeZoneNonUniformity | 0.231787764 | 0.535706218 | 0.897626166 |
| wavelet-HLL_glszm_SizeZoneNonUniformity | 0.233534798 | 0.563307794 | 0.897626166 |
| wavelet-HLH_glszm_GrayLevelNonUniformity | 0.235741376 | 0.392303814 | 0.897626166 |
| wavelet-LHL_glcm_Imc1 | 0.240111187 | 0.743670047 | 0.897626166 |
| wavelet-HHL_glszm_SizeZoneNonUniformityNormalized | 0.245442792 | 0.529411842 | 0.897626166 |
| wavelet-LHL_glcm_Idn | 0.25249541 | 0.493430259 | 0.897626166 |
| wavelet-LLH_glcm_Idn | 0.255079037 | 0.46007342 | 0.897626166 |
| wavelet-HLL_gldm_DependenceEntropy | 0.25930461 | 0.574077773 | 0.897626166 |
| wavelet-LLL_glcm_Idmn | 0.262622061 | 0.470252427 | 0.897626166 |
| wavelet-LHL_glszm_GrayLevelVariance | 0.264556416 | −0.677418131 | 0.897626166 |
| wavelet-HHL_glszm_ZonePercentage | 0.275233981 | −0.444249158 | 0.897626166 |
| wavelet-HHL_gldm_SmallDependenceEmphasis | 0.281389388 | −0.456240739 | 0.897626166 |
| wavelet-HLL_ngtdm_Busyness | 0.282270323 | −0.492514844 | 0.897626166 |
| wavelet-HHL_glrlm_GrayLevelNonUniformityNormalized | 0.294113291 | 0.415451994 | 0.897626166 |
| wavelet-LHL_ngtdm_Contrast | 0.297492541 | −0.457901927 | 0.897626166 |
| wavelet-HHL_glcm_DifferenceAverage | 0.300773893 | −0.467223104 | 0.897626166 |
| wavelet-LLH_glrlm_RunLengthNonUniformity | 0.308568206 | 0.40312166 | 0.897626166 |
| wavelet-HLL_glcm_Correlation | 0.310491405 | 0.407347514 | 0.897626166 |
| wavelet-HHL_glcm_Idm | 0.313034979 | 0.429854688 | 0.897626166 |
| wavelet-HHL_glcm_Id | 0.313352457 | 0.435282413 | 0.897626166 |
| wavelet-HHL_glcm_JointEnergy | 0.313771662 | 0.365409325 | 0.897626166 |
| wavelet-HLL_glrlm_RunLengthNonUniformity | 0.316174387 | 0.395456825 | 0.897626166 |
| wavelet-LHL_glrlm_LongRunHighGrayLevelEmphasis | 0.316753108 | 0.465844133 | 0.897626166 |
| wavelet-LLL_glszm_SmallAreaLowGrayLevelEmphasis | 0.318246057 | −0.509007241 | 0.897626166 |
| wavelet-LHL_gldm_LargeDependenceEmphasis | 0.325254557 | 0.409022287 | 0.897626166 |
| wavelet-LHL_gldm_LargeDependenceHighGrayLevelEmphasis | 0.325797148 | 0.413024225 | 0.897626166 |
| wavelet-LLL_gldm_SmallDependenceLowGrayLevelEmphasis | 0.327657484 | −0.449736489 | 0.897626166 |
| wavelet-LHL_glcm_Idm | 0.329282385 | 0.381179983 | 0.897626166 |
| wavelet-LHL_glcm_Id | 0.330743858 | 0.379087837 | 0.897626166 |
| wavelet-HLL_glrlm_GrayLevelVariance | 0.33084894 | 0.702882204 | 0.897626166 |
| wavelet-HHL_glcm_MCC | 0.333560351 | −0.486103378 | 0.897626166 |
| wavelet-HHL_glrlm_GrayLevelVariance | 0.334136795 | −0.416865548 | 0.897626166 |
| wavelet-LLH_glrlm_RunLengthNonUniformityNormalized | 0.33612835 | 0.399918297 | 0.897626166 |
| wavelet-LLH_glszm_ZoneEntropy | 0.337054304 | 0.565463316 | 0.897626166 |
| wavelet-HHL_glcm_Contrast | 0.337193865 | −0.453029288 | 0.897626166 |
| wavelet-HHL_gldm_LargeDependenceEmphasis | 0.338721987 | 0.444622667 | 0.897626166 |
| wavelet-HHL_gldm_DependenceEntropy | 0.339624473 | −0.535464387 | 0.897626166 |
| wavelet-LLH_glrlm_ShortRunEmphasis | 0.343713411 | 0.438500763 | 0.897626166 |
| wavelet-LHL_glrlm_RunPercentage | 0.346792517 | −0.428846492 | 0.897626166 |
| wavelet-HHH_glszm_SmallAreaHighGrayLevelEmphasis | 0.347474312 | 0.428224796 | 0.897626166 |
| wavelet-HLH_glcm_DifferenceAverage | 0.347653771 | −0.743254134 | 0.897626166 |
| wavelet-LHH_glcm_Idn | 0.349500772 | 0.458568119 | 0.897626166 |
| wavelet-LLL_gldm_DependenceEntropy | 0.350420899 | 0.38318276 | 0.897626166 |
| wavelet-HHL_glcm_DifferenceVariance | 0.35087509 | −0.415222211 | 0.897626166 |
| wavelet-LHH_glszm_SmallAreaEmphasis | 0.351586355 | 0.338815948 | 0.897626166 |
| wavelet-HHL_glcm_SumSquares | 0.353295371 | −0.42530631 | 0.897626166 |
| wavelet-HLL_glszm_LargeAreaHighGrayLevelEmphasis | 0.353678262 | 0.368250531 | 0.897626166 |
| wavelet-LHL_glcm_Idmn | 0.354159832 | 0.433171017 | 0.897626166 |
| wavelet-HHL_glrlm_RunPercentage | 0.355128303 | −0.450911529 | 0.897626166 |
| wavelet-LHH_glcm_DifferenceAverage | 0.356484148 | −0.641021916 | 0.897626166 |
| wavelet-HHL_glcm_JointEntropy | 0.358710389 | −0.415113543 | 0.897626166 |
| wavelet-LHL_glrlm_RunLengthNonUniformityNormalized | 0.358826007 | −0.431194681 | 0.897626166 |
| wavelet-HHL_glcm_DifferenceEntropy | 0.359173712 | −0.464413833 | 0.897626166 |
| wavelet-HLL_glcm_ClusterTendency | 0.359498774 | 0.418273818 | 0.897626166 |
| wavelet-LHL_glcm_DifferenceVariance | 0.360338947 | −0.448780698 | 0.897626166 |
| wavelet-LLL_ngtdm_Complexity | 0.361145355 | 0.442233242 | 0.897626166 |
| wavelet-HHL_gldm_GrayLevelVariance | 0.361742581 | −0.41678238 | 0.897626166 |
| wavelet-LLH_glrlm_RunPercentage | 0.362353936 | 0.373165385 | 0.897626166 |
| wavelet-LHL_glcm_DifferenceEntropy | 0.363697869 | −0.463463658 | 0.897626166 |
| wavelet-LLH_glszm_GrayLevelNonUniformityNormalized | 0.364839347 | −0.376202949 | 0.897626166 |
| wavelet-HLH_glrlm_ShortRunEmphasis | 0.36573155 | −0.568141663 | 0.897626166 |
| wavelet-HHH_glszm_SmallAreaLowGrayLevelEmphasis | 0.371391297 | 0.304254983 | 0.897626166 |
| wavelet-HHL_glcm_ClusterTendency | 0.37231451 | −0.406352773 | 0.897626166 |
| wavelet-LLL_glrlm_RunEntropy | 0.374464423 | 0.453788113 | 0.897626166 |
| wavelet-LLL_ngtdm_Contrast | 0.376317687 | −0.357718218 | 0.897626166 |
| wavelet-HHH_glszm_GrayLevelNonUniformityNormalized | 0.379102336 | 0.520461449 | 0.897626166 |
| wavelet-LHH_glcm_Idm | 0.379359872 | 0.497022212 | 0.897626166 |
| wavelet-LLL_gldm_DependenceNonUniformity | 0.379658528 | 0.337139915 | 0.897626166 |
| wavelet-LHL_gldm_SmallDependenceEmphasis | 0.381367174 | −0.540385085 | 0.897626166 |
| wavelet-LHL_glrlm_ShortRunEmphasis | 0.382282066 | −0.408719029 | 0.897626166 |
| wavelet-HLH_glszm_SizeZoneNonUniformity | 0.382379563 | 0.397492961 | 0.897626166 |
| wavelet-HLH_glcm_Idm | 0.384422544 | 0.505860344 | 0.897626166 |
| wavelet-HHL_gldm_LargeDependenceHighGrayLevelEmphasis | 0.386288982 | 0.658220611 | 0.897626166 |
| wavelet-LLL_glcm_Idn | 0.386423896 | 0.411476178 | 0.897626166 |
| wavelet-LLH_gldm_LargeDependenceEmphasis | 0.386508404 | −0.358543057 | 0.897626166 |
| wavelet-LHH_glcm_Id | 0.388959113 | 0.443892438 | 0.897626166 |
| wavelet-LHL_glcm_Contrast | 0.391253626 | −0.425466562 | 0.897626166 |
| wavelet-HHL_glcm_SumEntropy | 0.392924091 | −0.429854586 | 0.897626166 |
| wavelet-LLH_glcm_ClusterShade | 0.393154031 | −0.460375617 | 0.897626166 |
| wavelet-LHL_glszm_LargeAreaHighGrayLevelEmphasis | 0.393186403 | 0.404446156 | 0.897626166 |
| wavelet-HLL_glcm_SumEntropy | 0.395736883 | 0.409102998 | 0.897626166 |
| wavelet-LHL_glrlm_GrayLevelNonUniformityNormalized | 0.396598898 | 0.352301064 | 0.897626166 |
| wavelet-LHL_glrlm_GrayLevelVariance | 0.397693202 | −0.419843292 | 0.897626166 |
| wavelet-HLH_glcm_Id | 0.397861641 | 0.43999502 | 0.897626166 |
| wavelet-HLL_gldm_GrayLevelVariance | 0.398473716 | 0.363030158 | 0.897626166 |
| wavelet-LHL_gldm_DependenceNonUniformityNormalized | 0.399660564 | −0.433520409 | 0.897626166 |
| wavelet-LHL_glcm_Correlation | 0.401259737 | 0.38582433 | 0.897626166 |
| wavelet-LHL_glcm_DifferenceAverage | 0.403181515 | −0.442675653 | 0.897626166 |
| wavelet-HLL_glszm_ZoneVariance | 0.414155549 | −0.363842792 | 0.897626166 |
| wavelet-HLL_glszm_LargeAreaEmphasis | 0.414432788 | −0.364232656 | 0.897626166 |
| wavelet-HHL_glrlm_ShortRunEmphasis | 0.414492218 | −0.384663089 | 0.897626166 |
| wavelet-LHL_glrlm_RunLengthNonUniformity | 0.419277244 | 0.33366901 | 0.897626166 |
| wavelet-LLH_glrlm_GrayLevelNonUniformity | 0.419871662 | 0.320440567 | 0.897626166 |
| wavelet-LHL_glcm_JointEnergy | 0.422641863 | 0.292816277 | 0.897626166 |
| wavelet-HLL_glcm_SumSquares | 0.425260599 | 0.355763394 | 0.897626166 |
| wavelet-HHH_glszm_LowGrayLevelZoneEmphasis | 0.427579216 | −0.425401478 | 0.897626166 |
| wavelet-LLH_glszm_SmallAreaEmphasis | 0.428516623 | 0.386126451 | 0.897626166 |
| wavelet-LHH_glcm_InverseVariance | 0.429338775 | −0.328148942 | 0.897626166 |
| wavelet-LLL_glrlm_LongRunHighGrayLevelEmphasis | 0.432398054 | 0.390114222 | 0.897626166 |
| wavelet-LLL_glcm_Correlation | 0.433163494 | 0.375163225 | 0.897626166 |
| wavelet-LHL_gldm_GrayLevelVariance | 0.433341548 | −0.391841272 | 0.897626166 |
| wavelet-LHL_glcm_JointEntropy | 0.434379695 | −0.329917107 | 0.897626166 |
| wavelet-LHH_glrlm_LongRunLowGrayLevelEmphasis | 0.435305352 | −0.385262542 | 0.897626166 |
| wavelet-HLL_glcm_InverseVariance | 0.445518554 | −0.440176022 | 0.897626166 |
| wavelet-LHL_glszm_GrayLevelNonUniformity | 0.446032525 | 0.29943658 | 0.897626166 |
| wavelet-LHL_gldm_DependenceVariance | 0.446284096 | 0.47059151 | 0.897626166 |
| wavelet-LHL_gldm_GrayLevelNonUniformity | 0.454193943 | 0.275899007 | 0.897626166 |
| wavelet-HHL_glrlm_RunLengthNonUniformityNormalized | 0.455055773 | −0.37470425 | 0.897626166 |
| wavelet-HLH_glszm_SizeZoneNonUniformityNormalized | 0.459846861 | −0.300646355 | 0.897626166 |
| wavelet-LHL_glrlm_ShortRunLowGrayLevelEmphasis | 0.461890195 | −0.419287575 | 0.897626166 |
| wavelet-LHL_glcm_Autocorrelation | 0.462261978 | 0.385271258 | 0.897626166 |
| wavelet-LHL_glcm_SumSquares | 0.464613942 | −0.365350547 | 0.897626166 |
| wavelet-LHL_glszm_GrayLevelNonUniformityNormalized | 0.465496902 | 0.314039507 | 0.897626166 |
| wavelet-HHL_glrlm_GrayLevelNonUniformity | 0.468357585 | 0.248563508 | 0.897626166 |
| wavelet-LLH_glcm_MaximumProbability | 0.470348343 | −0.461108022 | 0.897626166 |
| wavelet-LHL_gldm_HighGrayLevelEmphasis | 0.471743464 | 0.376920024 | 0.897626166 |
| wavelet-LHL_glcm_MCC | 0.472527622 | −0.380894111 | 0.897626166 |
| wavelet-HHH_glrlm_RunLengthNonUniformity | 0.472979951 | 0.282628841 | 0.897626166 |
| wavelet-LHL_glszm_SizeZoneNonUniformity | 0.473651211 | 0.329956071 | 0.897626166 |
| wavelet-HHL_glszm_SmallAreaEmphasis | 0.475508419 | 0.383928146 | 0.897626166 |
| wavelet-HLL_glcm_JointEntropy | 0.47564865 | 0.326302631 | 0.897626166 |
| wavelet-LHL_glrlm_HighGrayLevelRunEmphasis | 0.476380509 | 0.371084974 | 0.897626166 |
| wavelet-LHL_glrlm_GrayLevelNonUniformity | 0.476560956 | 0.257377348 | 0.897626166 |
| wavelet-HHL_gldm_GrayLevelNonUniformity | 0.477838458 | 0.240531969 | 0.897626166 |
| wavelet-LLH_glcm_DifferenceAverage | 0.478299204 | 0.329113673 | 0.897626166 |
| wavelet-LLH_glcm_Idm | 0.480664565 | −0.327917429 | 0.897626166 |
| wavelet-LLL_glcm_Imc2 | 0.481167041 | 0.38422731 | 0.897626166 |
| wavelet-LLH_glcm_Id | 0.482070817 | −0.327225479 | 0.897626166 |
| wavelet-LLL_glcm_Imc1 | 0.482842465 | −0.353645174 | 0.897626166 |
| wavelet-HLH_glrlm_RunLengthNonUniformity | 0.484730127 | 0.277635362 | 0.897626166 |
| wavelet-HHL_glrlm_RunEntropy | 0.485864214 | −0.327584164 | 0.897626166 |
| wavelet-LLL_gldm_LargeDependenceHighGrayLevelEmphasis | 0.489674264 | 0.328008605 | 0.897626166 |
| wavelet-LLH_glszm_SizeZoneNonUniformityNormalized | 0.490433912 | 0.323104187 | 0.897626166 |
| wavelet-LLL_glcm_SumEntropy | 0.490554985 | 0.268280383 | 0.897626166 |
| wavelet-LLL_glcm_JointAverage | 0.49183793 | 0.360287234 | 0.897626166 |
| wavelet-LLL_glcm_SumAverage | 0.49183793 | 0.360287234 | 0.897626166 |
| wavelet-HHH_glrlm_GrayLevelNonUniformity | 0.495525218 | 0.264232054 | 0.897626166 |
| wavelet-LHL_glcm_JointAverage | 0.496352875 | 0.323253471 | 0.897626166 |
| wavelet-LHL_glcm_SumAverage | 0.496352875 | 0.323253471 | 0.897626166 |
| wavelet-HHL_gldm_SmallDependenceLowGrayLevelEmphasis | 0.497098557 | −0.356483675 | 0.897626166 |
| wavelet-LLL_glcm_MCC | 0.497201345 | 0.305188129 | 0.897626166 |
| wavelet-HLH_ngtdm_Busyness | 0.500910772 | 0.264297056 | 0.897626166 |
| wavelet-LLH_glcm_Imc2 | 0.500954779 | −0.341049095 | 0.897626166 |
| wavelet-HHH_ngtdm_Busyness | 0.503274415 | 0.257845309 | 0.897626166 |
| wavelet-HLH_glrlm_GrayLevelNonUniformity | 0.504781307 | 0.261060185 | 0.897626166 |
| wavelet-HHL_glszm_SizeZoneNonUniformity | 0.507748321 | 0.276208785 | 0.897626166 |
| wavelet-HLH_glcm_InverseVariance | 0.508243024 | −0.250165601 | 0.897626166 |
| wavelet-HHL_glcm_MaximumProbability | 0.509638635 | 0.294010357 | 0.897626166 |
| wavelet-LHH_glrlm_GrayLevelNonUniformity | 0.511535749 | 0.257218246 | 0.897626166 |
| wavelet-LHL_gldm_SmallDependenceLowGrayLevelEmphasis | 0.51370333 | −0.290665612 | 0.897626166 |
| wavelet-HLL_glrlm_GrayLevelNonUniformityNormalized | 0.514427219 | −0.302856498 | 0.897626166 |
| wavelet-HHL_glszm_GrayLevelNonUniformityNormalized | 0.514536355 | 0.284565911 | 0.897626166 |
| wavelet-HHH_gldm_GrayLevelNonUniformity | 0.514945515 | 0.251599429 | 0.897626166 |
| wavelet-HLH_gldm_GrayLevelNonUniformity | 0.515188333 | 0.251433963 | 0.897626166 |
| wavelet-HLH_glcm_JointEntropy | 0.515324393 | −0.422348373 | 0.897626166 |
| wavelet-LLL_glszm_HighGrayLevelZoneEmphasis | 0.515514213 | 0.344925313 | 0.897626166 |
| wavelet-LHH_gldm_GrayLevelNonUniformity | 0.515703417 | 0.251128508 | 0.897626166 |
| wavelet-HLL_glszm_GrayLevelNonUniformity | 0.517840427 | 0.263674379 | 0.897626166 |
| wavelet-LHH_glrlm_RunLengthNonUniformity | 0.51861976 | 0.259255235 | 0.897626166 |
| wavelet-LHH_glcm_Correlation | 0.521274932 | 0.284032002 | 0.897626166 |
| wavelet-LLH_gldm_GrayLevelNonUniformity | 0.522183438 | 0.247138043 | 0.897626166 |
| wavelet-LHH_glrlm_RunLengthNonUniformityNormalized | 0.524227579 | −0.463009269 | 0.897626166 |
| wavelet-LHL_glszm_ZonePercentage | 0.528459457 | −0.416119386 | 0.897626166 |
| wavelet-LLH_glcm_DifferenceEntropy | 0.531994664 | 0.323529187 | 0.897626166 |
| wavelet-HHL_glrlm_RunLengthNonUniformity | 0.538434694 | 0.268990482 | 0.897626166 |
| wavelet-HLL_glszm_ZonePercentage | 0.541821275 | 0.285594561 | 0.897626166 |
| wavelet-HLL_glcm_DifferenceEntropy | 0.542533471 | 0.319509301 | 0.897626166 |
| wavelet-LHL_glszm_HighGrayLevelZoneEmphasis | 0.543865753 | 0.301338257 | 0.897626166 |
| wavelet-HHL_gldm_DependenceNonUniformity | 0.54545667 | 0.202531056 | 0.897626166 |
| wavelet-HLL_glcm_Contrast | 0.546661094 | 0.268740731 | 0.897626166 |
| wavelet-HHL_glcm_ClusterProminence | 0.546690922 | −0.333736846 | 0.897626166 |
| wavelet-HLH_glcm_ClusterTendency | 0.54723815 | 0.229828955 | 0.897626166 |
| wavelet-HLH_glcm_ClusterProminence | 0.547896473 | 0.227555517 | 0.897626166 |
| wavelet-LLH_glszm_LargeAreaLowGrayLevelEmphasis | 0.548994843 | −0.274510656 | 0.897626166 |
| wavelet-LHH_ngtdm_Busyness | 0.55197571 | 0.232821631 | 0.897626166 |
| wavelet-LHL_glszm_SmallAreaEmphasis | 0.553062527 | 0.296560694 | 0.897626166 |
| wavelet-LLH_glcm_Imc1 | 0.553231382 | 0.336833822 | 0.897626166 |
| wavelet-LHH_glrlm_RunPercentage | 0.553645772 | −0.418692955 | 0.897626166 |
| wavelet-LLH_glcm_InverseVariance | 0.554436023 | 0.275099782 | 0.897626166 |
| wavelet-LHL_glcm_ClusterTendency | 0.557429004 | −0.280591332 | 0.897626166 |
| wavelet-LHL_glcm_SumEntropy | 0.558061991 | −0.271045249 | 0.897626166 |
| wavelet-HHL_ngtdm_Contrast | 0.558404434 | −0.260510738 | 0.897626166 |
| wavelet-HLL_gldm_DependenceNonUniformity | 0.559095611 | 0.221466735 | 0.897626166 |
| wavelet-LHH_gldm_LargeDependenceEmphasis | 0.560141571 | 0.376704725 | 0.897626166 |
| wavelet-LHL_glcm_MaximumProbability | 0.56065582 | 0.252748162 | 0.897626166 |
| wavelet-HLL_glcm_Imc1 | 0.562494377 | −0.408464089 | 0.897626166 |
| wavelet-HLH_glrlm_RunLengthNonUniformityNormalized | 0.565261001 | −0.381266622 | 0.897626166 |
| wavelet-HLH_glrlm_LongRunEmphasis | 0.56610199 | 0.280575268 | 0.897626166 |
| wavelet-HLH_glcm_Correlation | 0.568688414 | 0.247251762 | 0.897626166 |
| wavelet-HLH_glcm_MCC | 0.575164141 | −0.275223943 | 0.897626166 |
| wavelet-HHH_glrlm_LongRunLowGrayLevelEmphasis | 0.575645762 | −0.2200783 | 0.897626166 |
| wavelet-HLH_gldm_LargeDependenceEmphasis | 0.578709162 | 0.327361745 | 0.897626166 |
| wavelet-LLL_glcm_JointEntropy | 0.578970873 | 0.212166136 | 0.897626166 |
| wavelet-LLL_glrlm_HighGrayLevelRunEmphasis | 0.58274009 | 0.319681587 | 0.897626166 |
| wavelet-LLL_gldm_HighGrayLevelEmphasis | 0.583111431 | 0.320323093 | 0.897626166 |
| wavelet-LHL_ngtdm_Complexity | 0.583253839 | 0.377890315 | 0.897626166 |
| wavelet-HLH_glrlm_RunPercentage | 0.58519512 | −0.346157587 | 0.897626166 |
| wavelet-LHL_glrlm_ShortRunHighGrayLevelEmphasis | 0.586826807 | 0.281773686 | 0.897626166 |
| wavelet-HLL_glcm_JointEnergy | 0.588491364 | −0.256562814 | 0.897626166 |
| wavelet-LLH_glcm_Correlation | 0.590337099 | −0.292041362 | 0.897626166 |
| wavelet-LHL_glrlm_LongRunEmphasis | 0.591209961 | 0.241886253 | 0.897626166 |
| wavelet-HHL_glcm_Idn | 0.591501569 | 0.337322089 | 0.897626166 |
| wavelet-LLH_gldm_DependenceNonUniformity | 0.593809041 | 0.194703221 | 0.897626166 |
| wavelet-LLL_glcm_Autocorrelation | 0.595226086 | 0.311269736 | 0.897626166 |
| wavelet-HLL_glcm_DifferenceAverage | 0.596632662 | 0.289275622 | 0.897626166 |
| wavelet-HLH_gldm_DependenceNonUniformity | 0.597166306 | 0.205808288 | 0.897626166 |
| wavelet-HLL_glrlm_LongRunEmphasis | 0.600462882 | −0.228126411 | 0.897626166 |
| wavelet-LLL_glrlm_GrayLevelNonUniformityNormalized | 0.601505302 | −0.205846039 | 0.897626166 |
| wavelet-LHH_glrlm_ShortRunEmphasis | 0.603618519 | −0.365441514 | 0.897626166 |
| wavelet-LHL_glrlm_RunVariance | 0.60477378 | 0.229968356 | 0.897626166 |
| wavelet-LHH_gldm_DependenceNonUniformity | 0.607775823 | 0.199606782 | 0.897626166 |
| wavelet-HLL_glcm_Imc2 | 0.609297996 | 0.236210804 | 0.897626166 |
| wavelet-LLH_glrlm_LongRunEmphasis | 0.613563164 | −0.258538378 | 0.897626166 |
| wavelet-LHL_gldm_DependenceNonUniformity | 0.614150127 | 0.199297972 | 0.897626166 |
| wavelet-HHL_glcm_Idmn | 0.614806536 | 0.22173363 | 0.897626166 |
| wavelet-HHL_gldm_LargeDependenceLowGrayLevelEmphasis | 0.615575776 | 0.341760957 | 0.897626166 |
| wavelet-HLH_glrlm_LongRunLowGrayLevelEmphasis | 0.616947811 | 0.241649605 | 0.897626166 |
| wavelet-LLL_glrlm_LongRunLowGrayLevelEmphasis | 0.618576898 | −0.264737102 | 0.897626166 |
| wavelet-LLL_glrlm_ShortRunHighGrayLevelEmphasis | 0.621230733 | 0.286578103 | 0.897626166 |
| wavelet-HHH_gldm_DependenceNonUniformity | 0.621887936 | 0.186530653 | 0.897626166 |
| wavelet-HLL_gldm_DependenceVariance | 0.624097315 | 0.30138669 | 0.897626166 |
| wavelet-LLH_glszm_LargeAreaHighGrayLevelEmphasis | 0.624419582 | 0.247155191 | 0.897626166 |
| wavelet-LHH_glszm_LowGrayLevelZoneEmphasis | 0.624699291 | 0.250920082 | 0.897626166 |
| wavelet-LHH_glrlm_RunEntropy | 0.63867297 | 0.231486204 | 0.900590271 |
| wavelet-HLL_gldm_SmallDependenceEmphasis | 0.639926616 | 0.209828104 | 0.900590271 |
| wavelet-LHL_glszm_ZoneEntropy | 0.640938394 | 0.175096826 | 0.900590271 |
| wavelet-HLL_glrlm_GrayLevelNonUniformity | 0.643732947 | 0.179578154 | 0.900590271 |
| wavelet-HHL_glszm_ZoneEntropy | 0.645459918 | −0.280663903 | 0.900590271 |
| wavelet-HHH_glrlm_LongRunHighGrayLevelEmphasis | 0.647222983 | −0.185593094 | 0.900590271 |
| wavelet-HHL_glrlm_RunVariance | 0.648247806 | 0.206194448 | 0.900590271 |
| wavelet-HHL_glszm_SmallAreaHighGrayLevelEmphasis | 0.656493775 | 0.244606392 | 0.900590271 |
| wavelet-LLL_glszm_SizeZoneNonUniformity | 0.656921951 | 0.176113492 | 0.900590271 |
| wavelet-LHL_glszm_SmallAreaHighGrayLevelEmphasis | 0.659643439 | 0.243937691 | 0.900590271 |
| wavelet-HLH_glcm_Idn | 0.66027656 | 0.201623467 | 0.900590271 |
| wavelet-LHH_gldm_LargeDependenceLowGrayLevelEmphasis | 0.661086166 | −0.230130811 | 0.900590271 |
| wavelet-LLH_glrlm_RunVariance | 0.664080903 | −0.206888557 | 0.900590271 |
| wavelet-LLH_gldm_DependenceNonUniformityNormalized | 0.665472698 | −0.261974851 | 0.900590271 |
| wavelet-LLH_glcm_MCC | 0.666043176 | −0.242089761 | 0.900590271 |
| wavelet-HHL_glszm_SmallAreaLowGrayLevelEmphasis | 0.668677894 | 0.151867122 | 0.900590271 |
| wavelet-HLH_glcm_Imc2 | 0.668873888 | −0.201986174 | 0.900590271 |
| wavelet-HLL_glcm_Idm | 0.670925199 | −0.196941306 | 0.900590271 |
| wavelet-HHL_glszm_HighGrayLevelZoneEmphasis | 0.675997755 | 0.289448992 | 0.900590271 |
| wavelet-HHL_glrlm_LongRunHighGrayLevelEmphasis | 0.676629279 | 0.197449547 | 0.900590271 |
| wavelet-HLH_gldm_LargeDependenceLowGrayLevelEmphasis | 0.67709271 | 0.210946983 | 0.900590271 |
| wavelet-HLH_glrlm_RunEntropy | 0.679180286 | 0.198617484 | 0.900590271 |
| wavelet-LLL_ngtdm_Busyness | 0.679743826 | 0.174804108 | 0.900590271 |
| wavelet-HLL_glcm_Id | 0.680240331 | −0.197540402 | 0.900590271 |
| wavelet-HLL_glrlm_RunVariance | 0.681336404 | −0.173915801 | 0.900590271 |
| wavelet-LLL_gldm_LargeDependenceLowGrayLevelEmphasis | 0.683192817 | −0.209994371 | 0.900590271 |
| wavelet-LHL_glszm_SmallAreaLowGrayLevelEmphasis | 0.684047427 | −0.197638979 | 0.900590271 |
| wavelet-LLL_glrlm_GrayLevelNonUniformity | 0.685902546 | 0.167498416 | 0.900590271 |
| wavelet-HHL_glrlm_LongRunEmphasis | 0.686493617 | 0.189241563 | 0.900590271 |
| wavelet-LHL_glszm_LowGrayLevelZoneEmphasis | 0.687612842 | −0.150979807 | 0.900590271 |
| wavelet-LLL_glszm_SizeZoneNonUniformityNormalized | 0.692864343 | −0.203576839 | 0.903374166 |
| wavelet-HHL_gldm_SmallDependenceHighGrayLevelEmphasis | 0.693807636 | −0.196648069 | 0.903374166 |
| wavelet-HLH_glcm_SumEntropy | 0.69712361 | 0.160773932 | 0.90408382 |
| wavelet-LLH_glrlm_RunEntropy | 0.699327996 | −0.182119647 | 0.90408382 |
| wavelet-LHL_glrlm_LongRunLowGrayLevelEmphasis | 0.702013632 | −0.176110938 | 0.90408382 |
| wavelet-HLH_glszm_LowGrayLevelZoneEmphasis | 0.703353787 | −0.19842323 | 0.90408382 |
| wavelet-LLL_glszm_GrayLevelVariance | 0.704533788 | 0.158004517 | 0.90408382 |
| wavelet-LLL_glcm_JointEnergy | 0.722844972 | −0.175320681 | 0.918441758 |
| wavelet-HLL_glrlm_ShortRunEmphasis | 0.727888308 | 0.146619975 | 0.918441758 |
| wavelet-HLH_glcm_DifferenceEntropy | 0.727895682 | −0.212924248 | 0.918441758 |
| wavelet-HHL_glcm_Autocorrelation | 0.733705603 | 0.229645584 | 0.918441758 |
| wavelet-LLH_gldm_SmallDependenceEmphasis | 0.734063444 | 0.158368803 | 0.918441758 |
| wavelet-HLL_gldm_GrayLevelNonUniformity | 0.735792587 | 0.126252372 | 0.918441758 |
| wavelet-HHH_glrlm_LongRunEmphasis | 0.737929695 | −0.155861911 | 0.918441758 |
| wavelet-HLH_glszm_SmallAreaEmphasis | 0.744372757 | 0.121256574 | 0.918441758 |
| wavelet-HLL_glrlm_RunPercentage | 0.744834829 | 0.139008371 | 0.918441758 |
| wavelet-LLL_glcm_ClusterTendency | 0.745771053 | 0.145953017 | 0.918441758 |
| wavelet-HHH_gldm_LargeDependenceEmphasis | 0.748047565 | 0.186892748 | 0.918441758 |
| wavelet-LHH_gldm_DependenceEntropy | 0.748330529 | 0.206054891 | 0.918441758 |
| wavelet-HLL_glrlm_RunLengthNonUniformityNormalized | 0.748646294 | 0.138790593 | 0.918441758 |
| wavelet-HHL_gldm_HighGrayLevelEmphasis | 0.750335484 | 0.212740132 | 0.918441758 |
| wavelet-LLL_glcm_InverseVariance | 0.750741123 | −0.157791982 | 0.918441758 |
| wavelet-HHL_glrlm_HighGrayLevelRunEmphasis | 0.752548126 | 0.210238723 | 0.918441758 |
| wavelet-HLH_glcm_DifferenceVariance | 0.754238769 | −0.184421816 | 0.918441758 |
| wavelet-LHL_gldm_DependenceEntropy | 0.7553962 | −0.16688066 | 0.918441758 |
| wavelet-LLL_glcm_SumSquares | 0.760581553 | 0.134513192 | 0.918441758 |
| wavelet-LLH_ngtdm_Busyness | 0.763820621 | −0.139679933 | 0.918441758 |
| wavelet-HLH_glcm_Imc1 | 0.763871744 | −0.174618993 | 0.918441758 |
| wavelet-LLL_glcm_MaximumProbability | 0.766378144 | −0.121377688 | 0.918441758 |
| wavelet-LLL_glszm_SmallAreaHighGrayLevelEmphasis | 0.76928232 | 0.159712461 | 0.918441758 |
| wavelet-HHH_glrlm_RunPercentage | 0.769898639 | −0.175808712 | 0.918441758 |
| wavelet-LLL_glcm_ClusterProminence | 0.770591577 | 0.147875706 | 0.918441758 |
| wavelet-LHL_gldm_LowGrayLevelEmphasis | 0.77159082 | −0.107086078 | 0.918441758 |
| wavelet-LHL_glrlm_LowGrayLevelRunEmphasis | 0.772768178 | −0.107129762 | 0.918441758 |
| wavelet-LLL_gldm_GrayLevelNonUniformity | 0.774038045 | 0.114658635 | 0.918441758 |
| wavelet-HHH_glrlm_RunVariance | 0.775710944 | −0.157115175 | 0.918441758 |
| wavelet-HLL_glszm_SizeZoneNonUniformityNormalized | 0.78605752 | 0.140373597 | 0.928216859 |
| wavelet-HLH_glrlm_RunVariance | 0.789104894 | 0.140792506 | 0.928785197 |
| wavelet-LHH_glrlm_LongRunEmphasis | 0.791095168 | −0.144141504 | 0.928785197 |
| wavelet-HLL_gldm_LargeDependenceEmphasis | 0.792814391 | −0.117648189 | 0.928785197 |
| wavelet-HHL_glcm_Inverse_Variance | 0.801670791 | −0.141831921 | 0.936689029 |
| wavelet-HLL_glcm_MaximumProbability | 0.80566217 | −0.123303176 | 0.938881899 |
| wavelet-HHL_ngtdm_Busyness | 0.813695246 | 0.115303283 | 0.943753142 |
| wavelet-LLL_glrlm_GrayLevelVariance | 0.815780305 | 0.104289823 | 0.943753142 |
| wavelet-LLL_glcm_DifferenceEntropy | 0.819128957 | 0.103783298 | 0.943753142 |
| wavelet-HHL_glszm_GrayLevelNonUniformity | 0.824632492 | 0.09547967 | 0.943753142 |
| wavelet-LHH_gldm_DependenceNonUniformityNormalized | 0.826996457 | −0.108119031 | 0.943753142 |
| wavelet-HHH_gldm_LargeDependenceHighGrayLevelEmphasis | 0.829504716 | 0.096912109 | 0.943753142 |
| wavelet-LLL_glszm_ZonePercentage | 0.830083199 | −0.108670696 | 0.943753142 |
| wavelet-HLH_glcm_MaximumProbability | 0.830714461 | 0.180047108 | 0.943753142 |
| wavelet-HHH_glrlm_RunLengthNonUniformityNormalized | 0.831981517 | −0.130701678 | 0.943753142 |
| wavelet-LLL_glcm_ClusterShade | 0.832533271 | −0.099526316 | 0.943753142 |
| wavelet-HHL_ngtdm_Complexity | 0.833223494 | 0.108959243 | 0.943753142 |
| wavelet-LHL_glcm_ClusterProminence | 0.843469829 | −0.099801833 | 0.948174059 |
| wavelet-LHL_glszm_SizeZoneNonUniformityNormalized | 0.843635763 | 0.116423645 | 0.948174059 |
| wavelet-HHL_glrlm_LongRunLowGrayLevelEmphasis | 0.84492579 | 0.12140743 | 0.948174059 |
| wavelet-LLL_gldm_DependenceNonUniformityNormalized | 0.846124747 | 0.096551277 | 0.948174059 |
| wavelet-LLL_gldm_SmallDependenceEmphasis | 0.847804283 | −0.091820656 | 0.948174059 |
| wavelet-HLH_gldm_LowGrayLevelEmphasis | 0.859553858 | −0.113402856 | 0.957579419 |
| wavelet-LLL_gldm_GrayLevelVariance | 0.860527451 | 0.07848704 | 0.957579419 |
| wavelet-HLL_gldm_DependenceNonUniformityNormalized | 0.864878644 | −0.110641487 | 0.958480809 |
| wavelet-HHL_glcm_SumAverage | 0.872304807 | 0.091549223 | 0.958480809 |
| wavelet-HHL_glcm_JointAverage | 0.872304807 | 0.091549223 | 0.958480809 |
| wavelet-LLL_glcm_Idm | 0.875486711 | −0.070059832 | 0.958480809 |
| wavelet-HHH_glrlm_ShortRunLowGrayLevelEmphasis | 0.8756334 | 0.064471006 | 0.958480809 |
| wavelet-HHH_glrlm_RunEntropy | 0.881604793 | 0.062773314 | 0.958480809 |
| wavelet-LHL_gldm_SmallDependenceHighGrayLevelEmphasis | 0.883846806 | 0.086594356 | 0.958480809 |
| wavelet-LLL_glcm_DifferenceAverage | 0.885675739 | 0.074219811 | 0.958480809 |
| wavelet-LLH_glszm_Zone_Variance | 0.886299926 | −0.059799032 | 0.958480809 |
| wavelet-LLH_gldm_DependenceVariance | 0.886846058 | −0.092117481 | 0.958480809 |
| wavelet-LLL_glcm_Id | 0.887431532 | −0.064950244 | 0.958480809 |
| wavelet-LLL_glrlm_RunLengthNonUniformityNormalized | 0.889929301 | 0.059598371 | 0.958480809 |
| wavelet-LLL_glrlm_ShortRunEmphasis | 0.892060013 | 0.058591826 | 0.958480809 |
| wavelet-LLH_glszm_LargeAreaEmphasis | 0.892430009 | −0.056313411 | 0.958480809 |
| wavelet-LLL_glcm_Contrast | 0.893718593 | −0.078947899 | 0.958480809 |
| wavelet-LHL_glrlm_RunEntropy | 0.896050525 | −0.057238427 | 0.958666104 |
| wavelet-LHL_ngtdm_Busyness | 0.901103235 | −0.050259476 | 0.961184668 |
| wavelet-HHL_glrlm_ShortRunHighGrayLevelEmphasis | 0.902734249 | 0.074289966 | 0.961184668 |
| wavelet-LHH_glszm_SizeZoneNonUniformityNormalized | 0.906848822 | −0.045578477 | 0.962190905 |
| wavelet-HHH_glrlm_ShortRunEmphasis | 0.908196672 | −0.06313437 | 0.962190905 |
| wavelet-HHL_glrlm_ShortRunLowGrayLevelEmphasis | 0.912278635 | −0.042320545 | 0.962190905 |
| wavelet-LLH_glszm_SmallAreaLowGrayLevelEmphasis | 0.912347682 | −0.049740708 | 0.962190905 |
| wavelet-HLH_gldm_DependenceEntropy | 0.921887286 | 0.054789133 | 0.969209592 |
| wavelet-LLL_glrlm_RunPercentage | 0.923368598 | 0.042503803 | 0.969209592 |
| wavelet-HLH_gldm_DependenceNonUniformityNormalized | 0.926367009 | −0.043104234 | 0.970063566 |
| wavelet-LLL_gldm_SmallDependenceHighGrayLevelEmphasis | 0.930369357 | 0.042269281 | 0.97196234 |
| wavelet-LHH_gldm_DependenceVariance | 0.934321772 | 0.041097473 | 0.97279551 |
| wavelet-LLL_glszm_SmallAreaEmphasis | 0.935996481 | −0.045808248 | 0.97279551 |
| wavelet-LHH_glrlm_RunVariance | 0.937739816 | −0.040564491 | 0.97279551 |
| wavelet-HHH_gldm_DependenceVariance | 0.942759611 | 0.036456504 | 0.973154139 |
| wavelet-LLL_gldm_DependenceVariance | 0.943037321 | −0.034218053 | 0.973154139 |
| wavelet-HHH_gldm_DependenceEntropy | 0.944660887 | 0.031737103 | 0.973154139 |
| wavelet-LLL_glrlm_LongRunEmphasis | 0.948350533 | −0.032363989 | 0.974693603 |
| wavelet-LLL_glrlm_RunVariance | 0.957002481 | −0.027793275 | 0.981314322 |
| wavelet-LHL_glcm_InverseVariance | 0.964340315 | −0.023024656 | 0.983246303 |
| wavelet-HHL_gldm_LowGrayLevelEmphasis | 0.964765779 | 0.014254327 | 0.983246303 |
| wavelet-HHL_glszm_LowGrayLevelZoneEmphasis | 0.967612908 | 0.010936628 | 0.983246303 |
| wavelet-LLL_gldm_LargeDependenceEmphasis | 0.967744672 | −0.018054488 | 0.983246303 |
| wavelet-HLH_glszm_SmallAreaLowGrayLevelEmphasis | 0.970187873 | 0.014491768 | 0.983478118 |
| wavelet-LHL_gldm_LargeDependenceLowGrayLevelEmphasis | 0.975496592 | −0.010943201 | 0.985510386 |
| wavelet-HHH_gldm_DependenceNonUniformityNormalized | 0.976631914 | −0.013110114 | 0.985510386 |
| wavelet-HHH_gldm_LargeDependenceLowGrayLevelEmphasis | 0.980343331 | −0.0113306 | 0.987012334 |
| wavelet-HLH_gldm_DependenceVariance | 0.983624222 | −0.008542511 | 0.98807501 |
| wavelet-LLL_glcm_DifferenceVariance | 0.988361754 | −0.009309735 | 0.990592819 |
| wavelet-HHL_glrlm_LowGrayLevelRunEmphasis | 0.99695826 | 0.00123875 | 0.99695826 |

SUPPLEMENTARY TABLE 5

| feat | p | stat | p_corrected |
|---|---|---|---|
| Tumor_Other_mean_nuclear_area | 0.005707303 | 1.431149346 | 0.191583441 |
| Tumor_Other_quantile0.6_nuclear_area | 0.006349361 | 1.485730616 | 0.191583441 |
| Tumor_Other_quantile0.7_nuclear_area | 0.006785876 | 1.373847916 | 0.191583441 |
| Tumor_Other_quantile0.5_nuclear_area | 0.006862418 | 1.552375133 | 0.191583441 |
| Tumor_Other_quantile0.8_nuclear_area | 0.007586252 | 1.330074321 | 0.191583441 |
| Tumor_Other_quantile0.4_nuclear_area | 0.007931145 | 1.576610135 | 0.191583441 |
| Tumor_Other_quantile0.3_nuclear_area | 0.008911265 | 1.282287864 | 0.191583441 |
| Tumor_Other_quantile0.9_nuclear_area | 0.009389325 | 1.363426024 | 0.191583441 |
| Tumor_Other_quantile0.3_nuclear_max_diameter | 0.009892758 | 1.391708282 | 0.191583441 |
| Tumor_Other_quantile0.4_nuclear_max_diameter | 0.009967308 | 1.38452574 | 0.191583441 |
| Tumor_Other_mean_nuclear_max_diameter | 0.010502764 | 1.272476796 | 0.191583441 |
| Tumor_Other_quantile0.5_nuclear_max_diameter | 0.011626986 | 1.30324092 | 0.191583441 |
| Tumor_Other_quantile0.2_nuclear_max_diameter | 0.011644973 | 1.355766674 | 0.191583441 |
| Tumor_Other_quantile0.2_nuclear_area | 0.012417445 | 1.15793176 | 0.191583441 |
| Tumor_Other_quantile0.6_nuclear_max_diameter | 0.014115685 | 1.249235338 | 0.202508897 |
| Tumor_Other_quantile0.7_nuclear_max_diameter | 0.01695497 | 1.183758734 | 0.202508897 |
| Tumor_Other_quantile0.8_nuclear_max_diameter | 0.01707165 | 1.167139595 | 0.202508897 |
| Tumor_Other_quantile0.1_nuclear_area | 0.017088321 | 1.114416462 | 0.202508897 |
| Tumor_Other_quantile0.1_nuclear_max_diameter | 0.017813283 | 1.359560349 | 0.202508897 |
| Tumor_Other_quantile0.9_nuclear_max_diameter | 0.019108877 | 1.252783613 | 0.204854986 |
| Tumor_Other_skew_nuclear_hematoxylin_min | 0.019916457 | 1.016475739 | 0.204854986 |
| Stroma_major_axis_length | 0.028229096 | −0.758978781 | 0.277158399 |
| Tumor_Other_quantile0.1_nuclear_hematoxylin_stdDev | 0.037517924 | 0.888483635 | 0.352342241 |
| Necrosis_perimeter | 0.041990361 | 0.69770588 | 0.377913252 |
| Tumor_Other_var_nuclear_hematoxylin_median | 0.053614507 | 0.963976518 | 0.46322934 |
| Tumor_Other_quantile0.2_nuclear_hematoxylin_stdDev | 0.057486831 | 0.809236665 | 0.476209285 |
| Tumor_Other_var_nuclear_eosin_max | 0.059526161 | −0.980112541 | 0.476209285 |
| Necrosis_major_axis_length | 0.062264702 | −0.840602314 | 0.480327703 |
| Tumor_Other_quantile0.3_nuclear_hematoxylin_stdDev | 0.067306683 | 0.774062179 | 0.500241544 |
| Necrosis_eccentricity | 0.070262822 | −0.683322615 | 0.500241544 |
| Necrosis_solidity | 0.071793925 | 0.723665909 | 0.500241544 |
| Necrosis_extent | 0.076130092 | 0.75084161 | 0.513878118 |
| Tumor_major_axis_length | 0.078597077 | −0.702861109 | 0.514453597 |
| Tumor_Other_quantile0.4_nuclear_hematoxylin_stdDev | 0.097874082 | 0.694792617 | 0.61428451 |
| Tumor_Other_var_nuclear_hematoxylin_mean | 0.099536842 | 0.815261794 | 0.61428451 |
| Necrosis_Other_density | 0.103959413 | 0.628983674 | 0.623756476 |
| Tumor_Stroma_shannon_entropy | 0.12821683 | 0.54331979 | 0.743735572 |
| Tumor_Other_quantile0.5_nuclear_hematoxylin_stdDev | 0.131299605 | 0.627266245 | 0.743735572 |
| Necrosis_equivalent_diameter | 0.134285589 | 0.626573211 | 0.743735572 |
| Tumor_Other_quantile0.3_nuclear_eosin_min | 0.142744725 | −0.639655872 | 0.759176824 |
| Tumor_Other_quantile0.2_nuclear_eosin_min | 0.144103008 | −0.649062683 | 0.759176824 |
| Tumor_Other_quantile0.2_nuclear_hematoxylin_min | 0.152271187 | −0.783076848 | 0.762113997 |
| Tumor_Other_quantile0.3_nuclear_hematoxylin_min | 0.154828589 | −0.740705514 | 0.762113997 |
| Tumor_Other_quantile0.1_nuclear_hematoxylin_min | 0.155245444 | −0.893427388 | 0.762113997 |
| Tumor_Other_quantile0.4_nuclear_hematoxylin_min | 0.171040398 | −0.695665431 | 0.813444434 |
| Tumor_Other_quantile0.4_nuclear_eosin_min | 0.173233537 | −0.58865048 | 0.813444434 |
| Tumor_Other_mean_nuclear_hematoxylin_stdDev | 0.180692823 | 0.548541024 | 0.820638942 |
| Tumor_Other_quantile0.6_nuclear_hematoxylin_stdDev | 0.182364209 | 0.548043632 | 0.820638942 |
| Tumor_Other_quantile0.5_nuclear_hematoxylin_min | 0.189465155 | −0.655555575 | 0.835193337 |
| Tumor_Other_quantile0.1_nuclear_eosin_min | 0.209227627 | −0.594454013 | 0.882099128 |
| Stroma_largest_component_extent | 0.214342564 | 0.294949837 | 0.882099128 |
| Tumor_Other_quantile0.5_nuclear_eosin_min | 0.219279575 | −0.519658501 | 0.882099128 |
| Tumor_Other_quantile0.6_nuclear_hematoxylin_min | 0.221040282 | −0.600039806 | 0.882099128 |
| Stroma_eccentricity | 0.222277285 | −0.436704291 | 0.882099128 |
| Necrosis_largest_component_PA_ratio | 0.224608574 | 0.409695725 | 0.882099128 |
| Necrosis_area | 0.231316127 | 0.450942667 | 0.892219348 |
| Tumor_Other_quantile0.7_nuclear_hematoxylin_stdDev | 0.242672093 | 0.485633291 | 0.917128883 |
| Tumor_largest_component_eccentricity | 0.250995243 | −0.210164613 | 0.917128883 |
| Stroma_largest_component_major_axis_length | 0.252209871 | −0.77020862 | 0.917128883 |
| Tumor_solidity | 0.257682815 | 0.438766708 | 0.917128883 |
| Tumor_Other_var_nuclear_max_diameter | 0.25900399 | 0.701107405 | 0.917128883 |
| Tumor_Other_mean_nuclear_hematoxylin_min | 0.266811522 | −0.547398158 | 0.918577107 |
| Tumor_Other_quantile0.7_nuclear_hematoxylin_min | 0.267918323 | −0.529851273 | 0.918577107 |
| Stroma_convex_area | 0.277547309 | −0.440335607 | 0.936722169 |
| Tumor_Other_quantile0.6_nuclear_eosin_min | 0.2906633 | −0.435251334 | 0.948973322 |
| Stroma_largest_component_solidity | 0.29310454 | 0.281484361 | 0.948973322 |
| Stroma_largest_component_PA_ratio | 0.294357466 | 0.38240946 | 0.948973322 |
| Tumor_Other_skew_nuclear_eosin_max | 0.307263207 | −0.637408129 | 0.966363154 |
| Necrosis_convex_area | 0.308699341 | −0.431054023 | 0.966363154 |
| Tumor_extent | 0.315565654 | 0.422245389 | 0.973745447 |
| Tumor_Other_quantile0.8_nuclear_hematoxylin_stdDev | 0.326286931 | 0.451203743 | 0.989938054 |
| Tumor_Other_var_nuclear_eosin_min | 0.348219212 | 0.605481886 | 0.989938054 |
| Tumor_Other_mean_nuclear_eosin_min | 0.355663131 | −0.372901927 | 0.989938054 |
| Tumor_Other_quantile0.8_nuclear_hematoxylin_min | 0.359968556 | −0.431947959 | 0.989938054 |
| Tumor_Other_quantile0.7_nuclear_eosin_min | 0.372777498 | −0.360097953 | 0.989938054 |
| Stroma_largest_component_eccentricity | 0.385942318 | −0.151504712 | 0.989938054 |
| Tumor_convex_area | 0.388179386 | −0.346819593 | 0.989938054 |
| Tumor_Other_quantile0.2_nuclear_solidity | 0.407351661 | −0.256597519 | 0.989938054 |
| Tumor_Other_quantile0.1_nuclear_eosin_stdDev | 0.408579428 | 0.347758381 | 0.989938054 |
| Tumor_Other_skew_nuclear_area | 0.417423007 | −0.326389076 | 0.989938054 |
| Necrosis_largest_component_eccentricity | 0.422620096 | −0.148423523 | 0.989938054 |
| Stroma_minor_axis_length | 0.429647498 | −0.294531716 | 0.989938054 |
| Tumor_Other_var_nuclear_eosin_stdDev | 0.444162337 | −0.427579247 | 0.989938054 |
| Tumor_Other_quantile0.9_nuclear_eosin_max | 0.444402974 | −0.279360662 | 0.989938054 |
| Tumor_Other_quantile0._1_nuclear_hematoxylin_max | 0.447314938 | 0.299614192 | 0.989938054 |
| Tumor_Other_skew_nuclear_eosin_min | 0.449235603 | 0.367717937 | 0.989938054 |
| Tumor_Other_kurtosis_nuclear_eosin_max | 0.464974526 | −0.412566158 | 0.989938054 |
| Tumor_Other_quantile0.3_nuclear_hematoxylin_max | 0.47547866 | 0.27179264 | 0.989938054 |
| Tumor_perimeter | 0.475712752 | 0.23242146 | 0.989938054 |
| Tumor_Other_quantile0.9_nuclear_hematoxylin_stdDev | 0.48054238 | 0.346588527 | 0.989938054 |
| Tumor_Other_quantile0.8_nuclear_eosin_min | 0.483626399 | −0.275788216 | 0.989938054 |
| Tumor_Other_quantile0.2_nuclear_hematoxylin_max | 0.484955554 | 0.26990673 | 0.989938054 |
| Tumor_Other_quantile0.2_nuclear_eosin_stdDev | 0.490194492 | 0.282697344 | 0.989938054 |
| Tumor_Other_quantile0.1_nuclear_hematoxylin_median | 0.496594975 | −0.30525583 | 0.989938054 |
| Tumor_Other_quantile0.4_nuclear_hematoxylin_max | 0.499661767 | 0.253482402 | 0.989938054 |
| Tumor_eccentricity | 0.500505028 | −0.267092743 | 0.989938054 |
| Tumor_Other_skew_nuclear_circularity | 0.508463761 | 0.319405576 | 0.989938054 |
| Tumor_Other_quantile0.5_nuclear_hematoxylin_max | 0.511756522 | 0.246200961 | 0.989938054 |
| Tumor_Other_quantile0.9_nuclear_hematoxylin_min | 0.515210596 | −0.299124466 | 0.989938054 |
| Stroma_Lymphocyte_density | 0.52251929 | 0.267872712 | 0.989938054 |
| Tumor_Other_kurtosis_nuclear_hematoxylin_stdDev | 0.532972553 | 0.382875436 | 0.989938054 |
| Tumor_Other_var_nuclear_hematoxylin_stdDev | 0.534274266 | −0.383899733 | 0.989938054 |
| Tumor_Other_quantile0.6_nuclear_hematoxylin_max | 0.547343897 | 0.226404763 | 0.989938054 |
| Tumor_Other_quantile0.2_nuclear_hematoxylin_median | 0.551061687 | −0.256129421 | 0.989938054 |
| Stroma_euler_number | 0.559301275 | 0.235931464 | 0.989938054 |
| Tumor_Other_quantile0.3_nuclear_eosin_stdDev | 0.562205351 | 0.231872458 | 0.989938054 |
| Tumor_Other_kurtosis_nuclear_hematoxylin_max | 0.570648673 | 0.348072521 | 0.989938054 |
| Tumor_Other_quantile0.8_nuclear_eosin_max | 0.582449671 | −0.195930169 | 0.989938054 |
| Tumor_Other_quantile0.1_nuclear_hematoxylin_mean | 0.586831048 | −0.244197857 | 0.989938054 |
| Tumor_Other_quantile0.7_nuclear_hematoxylin_max | 0.588779757 | 0.204885994 | 0.989938054 |
| Tumor_Other_quantile0.9_nuclear_circularity | 0.601303428 | 0.262927704 | 0.989938054 |
| Tumor_Other_kurtosis_nuclear_eosin_stdDev | 0.606755554 | −0.25705589 | 0.989938054 |
| Tumor_Other_quantile0.4_nuclear_eosin_stdDev | 0.615453504 | 0.196184006 | 0.989938054 |
| Tumor_Other_quantile0.3_nuclear_hematoxylin_median | 0.61767225 | −0.208403734 | 0.989938054 |
| Tumor_Other_quantile0.2_nuclear_hematoxylin_mean | 0.618607202 | −0.21425055 | 0.989938054 |
| Tumor_minor_axis_length | 0.622038679 | −0.202394382 | 0.989938054 |
| Tumor_Other_mean_nuclear_hematoxylin_max | 0.627797161 | 0.183778076 | 0.989938054 |
| Tumor_Other_quantile0.9_nuclear_eosin_min | 0.630993234 | −0.181325438 | 0.989938054 |
| Tumor_Other_kurtosis_nuclear_solidity | 0.639698747 | −0.198962171 | 0.989938054 |
| Tumor_Other_skew_nuclear_hematoxylin_mean | 0.643250387 | 0.255093659 | 0.989938054 |
| Tumor_Other_quantile0.3_nuclear_solidity | 0.65728263 | −0.135760994 | 0.989938054 |
| Tumor_Other_kurtosis_nuclear_circularity | 0.670090347 | −0.189331485 | 0.989938054 |
| Necrosis_minor_axis_length | 0.673146846 | −0.155124214 | 0.989938054 |
| Tumor_Other_quantile0.4_nuclear_hematoxylin_median | 0.673674448 | −0.172420802 | 0.989938054 |
| Stroma_Other_density | 0.673697072 | 0.181163772 | 0.989938054 |
| Tumor_Other_quantile0.8_nuclear_hematoxylin_max | 0.677816557 | 0.158690535 | 0.989938054 |
| Tumor_Other_quantile0.3_nuclear_hematoxylin_mean | 0.678339955 | −0.173779941 | 0.989938054 |
| Stroma_PA_ratio | 0.681845066 | 0.161902331 | 0.989938054 |
| Tumor_Other_quantile0.5_nuclear_eosin_stdDev | 0.682113752 | 0.156035566 | 0.989938054 |
| Stroma_solidity | 0.686552086 | 0.152830903 | 0.989938054 |
| Tumor_Other_quantile0.7_nuclear_eosin_max | 0.692872118 | −0.140799401 | 0.989938054 |
| Tumor_Other_quantile0.8_nuclear_circularity | 0.700509748 | 0.186325408 | 0.989938054 |
| Tumor_Other_quantile0.1_nuclear_eosin_mean | 0.714686873 | −0.121684927 | 0.989938054 |
| Stroma_area | 0.714700321 | −0.142408756 | 0.989938054 |
| Tumor_largest_component_extent | 0.721900164 | 0.1042364 | 0.989938054 |
| Tumor_Other_mean_nuclear_eosin_max | 0.722029776 | −0.128615344 | 0.989938054 |
| Tumor_Other_quantile0.4_nuclear_hematoxylin_mean | 0.723088233 | −0.145793512 | 0.989938054 |
| Tumor_Other_skew_nuclear_hematoxylin_max | 0.731229832 | −0.218668954 | 0.989938054 |
| Tumor_Other_mean_nuclear_eosin_stdDev | 0.73164064 | 0.128914976 | 0.989938054 |
| Tumor_Other_quantile0.6_nuclear_eosin_stdDev | 0.736476503 | 0.125039924 | 0.989938054 |
| Necrosis_largest_component_major_axis_length | 0.73808888 | −0.133030999 | 0.989938054 |
| Tumor_Other_skew_nuclear_solidity | 0.7424723 | 0.135650325 | 0.989938054 |
| Tumor_PA_ratio | 0.747561873 | 0.127327747 | 0.989938054 |
| Tumor_Other_skew_nuclear_hematoxylin_median | 0.756719246 | 0.174257116 | 0.989938054 |
| Tumor_Other_quantile0.2_nuclear_circularity | 0.759037856 | −0.145240964 | 0.989938054 |
| Tumor_Other_quantile0.1_nuclear_eosin_median | 0.759731499 | −0.101325053 | 0.989938054 |
| Tumor_Other_quantile0.3_nuclear_circularity | 0.765960236 | −0.145327592 | 0.989938054 |
| Tumor_area | 0.766226804 | 0.119040358 | 0.989938054 |
| Tumor_Other_quantile0.5_nuclear_hematoxylin_median | 0.771219377 | −0.117438993 | 0.989938054 |
| Tumor_Other_quantile0.6_nuclear_eosin_max | 0.779397976 | −0.100023856 | 0.989938054 |
| Tumor_Other_quantile0.4_nuclear_circularity | 0.781681036 | −0.135642673 | 0.989938054 |
| Tumor_Other_quantile0.7_nuclear_eosin_stdDev | 0.794521467 | 0.098605545 | 0.989938054 |
| ratio_Tumor_Lymphocyte_to_Tumor_Other | 0.802819977 | −0.124946381 | 0.989938054 |
| Tumor_Other_quantile0.5_nuclear_hematoxylin_mean | 0.803571922 | −0.101021262 | 0.989938054 |
| Tumor_Other_quantile0.2_nuclear_eosin_mean | 0.805552337 | −0.077939271 | 0.989938054 |
| Tumor_Other_mean_nuclear_hematoxylin_median | 0.81142185 | −0.097849885 | 0.989938054 |
| Tumor_largest_component_PA_ratio | 0.815962262 | 0.08045769 | 0.989938054 |
| Tumor_Other_quantile0.9_nuclear_hematoxylin_max | 0.821813266 | 0.098695523 | 0.989938054 |
| Stroma_perimeter | 0.82448147 | 0.08136455 | 0.989938054 |
| Tumor_Other_quantile0.9_nuclear_hematoxylin_median | 0.824627037 | 0.088365978 | 0.989938054 |
| Tumor_Other_skew_nuclear_eosin_stdDev | 0.825248158 | −0.111096342 | 0.989938054 |
| Tumor_equivalent_diameter | 0.830127027 | 0.082112007 | 0.989938054 |
| Tumor_Other_var_nuclear_hematoxylin_max | 0.835686084 | 0.105678547 | 0.989938054 |
| Tumor_Other_skew_nuclear_hematoxylin_stdDev | 0.838938871 | −0.109994379 | 0.989938054 |
| Tumor_Other_density | 0.840795693 | 0.085857615 | 0.989938054 |
| Tumor_Other_quantile0.3_nuclear_eosin_mean | 0.840848844 | −0.06352077 | 0.989938054 |
| Tumor_Other_quantile0.9_nuclear_hematoxylin_mean | 0.841527724 | 0.079659629 | 0.989938054 |
| Tumor_Other_quantile0.1_nuclear_circularity | 0.84407966 | −0.10216536 | 0.989938054 |
| Tumor_Other_mean_nuclear_hematoxylin_mean | 0.849768308 | −0.077847089 | 0.989938054 |
| Tumor_Other_quantile0.5_nuclear_eosin_max | 0.852081999 | −0.066605126 | 0.989938054 |
| Tumor_Other_quantile0.7_nuclear_circularity | 0.854808862 | 0.088158885 | 0.989938054 |
| Tumor_Other_quantile0.8_nuclear_eosin_stdDev | 0.856173271 | 0.069737831 | 0.989938054 |
| Tumor_Other_quantile0.2_nuclear_eosin_median | 0.862276104 | −0.055061364 | 0.989938054 |
| Stroma_equivalent_diameter | 0.863268837 | −0.060282541 | 0.989938054 |
| Tumor_Other_quantile0.6_nuclear_hematoxylin_median | 0.863296723 | −0.068796319 | 0.989938054 |
| Tumor_Other_quantile0.1_nuclear_solidity | 0.864383983 | −0.064145832 | 0.989938054 |
| Tumor_Other_quantile0.4_nuclear_eosin_mean | 0.866379417 | −0.053828447 | 0.989938054 |
| Tumor_Other_kurtosis_nuclear_eosin_min | 0.874725673 | 0.104026015 | 0.989938054 |
| Tumor_Other_quantile0.9_nuclear_eosin_mean | 0.875797337 | −0.065262744 | 0.989938054 |
| Tumor_Other_mean_nuclear_eosin_mean | 0.876692796 | −0.052564345 | 0.989938054 |
| Tumor_Other_quantile0.6_nuclear_hematoxylin_mean | 0.885301162 | −0.058007967 | 0.989938054 |
| Tumor_Other_var_nuclear_eosin_median | 0.885567574 | 0.073502081 | 0.989938054 |
| Tumor_largest_component_solidity | 0.887786752 | −0.044751221 | 0.989938054 |
| Tumor_Other_quantile0.5_nuclear_eosin_mean | 0.895097248 | −0.042666353 | 0.989938054 |
| Tumor_Other_quantile0.5_nuclear_circularity | 0.89694091 | −0.060766487 | 0.989938054 |
| Tumor_Other_quantile0.3_nuclear_eosin_median | 0.903549876 | −0.038803977 | 0.989938054 |
| Tumor_Other_quantile0.8_nuclear_eosin_mean | 0.905698673 | −0.041836431 | 0.989938054 |
| Tumor_Other_quantile0.6_nuclear_eosin_mean | 0.908942329 | −0.037565464 | 0.989938054 |
| Tumor_Other_quantile0.4_nuclear_eosin_max | 0.916046239 | −0.037675752 | 0.989938054 |
| Tumor_Other_quantile0.7_nuclear_eosin_mean | 0.916844778 | −0.034997562 | 0.989938054 |
| Stroma_extent | 0.920549404 | 0.038087979 | 0.989938054 |
| Tumor_Other_quantile0.9_nuclear_eosin_stdDev | 0.924090374 | 0.038103434 | 0.989938054 |
| Tumor_Other_var_nuclear_solidity | 0.925122084 | −0.041539705 | 0.989938054 |
| Tumor_Other_var_nuclear_circularity | 0.926247413 | 0.053504991 | 0.989938054 |
| Tumor_Other_quantile0.8_nuclear_hematoxylin_mean | 0.92684537 | 0.036192927 | 0.989938054 |
| Tumor_Other_quantile0.8_nuclear_hematoxylin_median | 0.928907843 | 0.034947852 | 0.989938054 |
| Tumor_Other_quantile0.4_nuclear_eosin_median | 0.932222358 | −0.027480523 | 0.989938054 |
| Tumor_Other_mean_nuclear_solidity | 0.939391336 | −0.036120194 | 0.989938054 |
| Tumor_Other_mean_nuclear_circularity | 0.941957199 | −0.035352306 | 0.989938054 |
| Tumor_Other_mean_nuclear_eosin_median | 0.943940534 | −0.024309682 | 0.989938054 |
| Necrosis_PA_ratio | 0.944094501 | −0.030105587 | 0.989938054 |
| Tumor_Other_quantile0.9_nuclear_eosin_median | 0.946405627 | −0.028522575 | 0.989938054 |
| Tumor_Other_quantile0.1_nuclear_eosin_max | 0.950497117 | 0.022217001 | 0.989938054 |
| Tumor_Lymphocyte_density | 0.954585975 | −0.025259875 | 0.989938054 |
| Tumor_Other_var_nuclear_hematoxylin_min | 0.96639957 | 0.016827499 | 0.989938054 |
| Tumor_Other_quantile0.5_nuclear_eosin_median | 0.967331802 | −0.013544122 | 0.989938054 |
| Tumor_Other_quantile0.7_nuclear_hematoxylin_median | 0.969480748 | −0.015138959 | 0.989938054 |
| Tumor_Other_quantile0.2_nuclear_eosin_max | 0.972754721 | 0.012223032 | 0.989938054 |
| Tumor_Other_quantile0.6_nuclear_circularity | 0.977117622 | 0.013601348 | 0.989938054 |
| Tumor_Other_quantile0.3_nuclear_eosin_max | 0.980616532 | −0.008689589 | 0.989938054 |
| Tumor_Other_var_nuclear_eosin_mean | 0.982108968 | −0.010912968 | 0.989938054 |
| Tumor_Other_quantile0.8_nuclear_eosin_median | 0.983013505 | −0.007663606 | 0.989938054 |
| Tumor_Other_quantile0.7_nuclear_hematoxylin_mean | 0.984125665 | −0.007926028 | 0.989938054 |
| Tumor_Other_quantile0.6_nuclear_eosin_median | 0.984446159 | −0.00652998 | 0.989938054 |
| Tumor_euler_number | 0.985355007 | −0.007685143 | 0.989938054 |
| Tumor_Other_quantile0.7_nuclear_eosin_median | 0.993472736 | −0.002778959 | 0.993472736 |

SUPPLEMENTARY TABLE 6

| feat | p | stat |
|---|---|---|
| cgr | 0.001566931 | −0.702299907 |
| parp_nact | 0.01142331 | 0.640477036 |
| adnexal_lesion | 0.041858501 | −0.46478622 |
| omental_lesion | 0.14907851 | 0.399141871 |
| age | 0.165468665 | 0.720036314 |
| stage_II | 0.254374139 | 1.152559534 |
| stage_IV | 0.449482546 | −0.146320509 |
| stage_III | 0.531589216 | 0.12090577 |
| Type of surgery_NACT-IDS | 0.764360563 | −0.063599163 |
| Type of surgery_PDS | 0.764360563 | 0.063599163 |
| stage_I | 1 | 0 |
| stage_nan | 1 | 0 |
| Type of surgery_nan | 1 | 0 |

SUPPLEMENTARY TABLE 7

| | Number of scans |
|---|---|
| Exclusion criterion | (of 445 reviewed) |
| Post-operative | 41 |
| Poor signal-to-noise ratio | 20 |
| No clinical data (in TCGA CDR) | 14 |
| No omental/adnexal lesion, or only on 1 slice | 11 |
| Poor contrast bolus timing (not | 9 |
| portal venous phase) or non-contrast scan | |
| Beam hardening artifact | 4 |
| Motion artifact | 3 |
| File corruption | 3 |
| Incomplete A/P CT scan | 2 |

### B. Systems and Methods of Determining Risk Scores Using Multimodal Features

A diagnostics platform may evaluate a subject at risk of a certain condition (e.g., cancer, disease, or ailment) using prognostic information for the conditions, such as genetic sequencing data for the subject. The reliance on prognostic information alone, however, may yield poor prognosis and variable response to treatment. This may also lead to wasted computer resources on the platform from calculating and providing poor results. To address these and other technical challenges, a computing system may combine features from disparate sources, such as histopathological data, radiomic data, and genomic data. The computing system may establish a multivariate model using these combined features to improve prediction of treatment response in accordance with machine learning (ML) techniques. In this manner, in providing more accurate and useful results, the computing system may reduce computer resources.

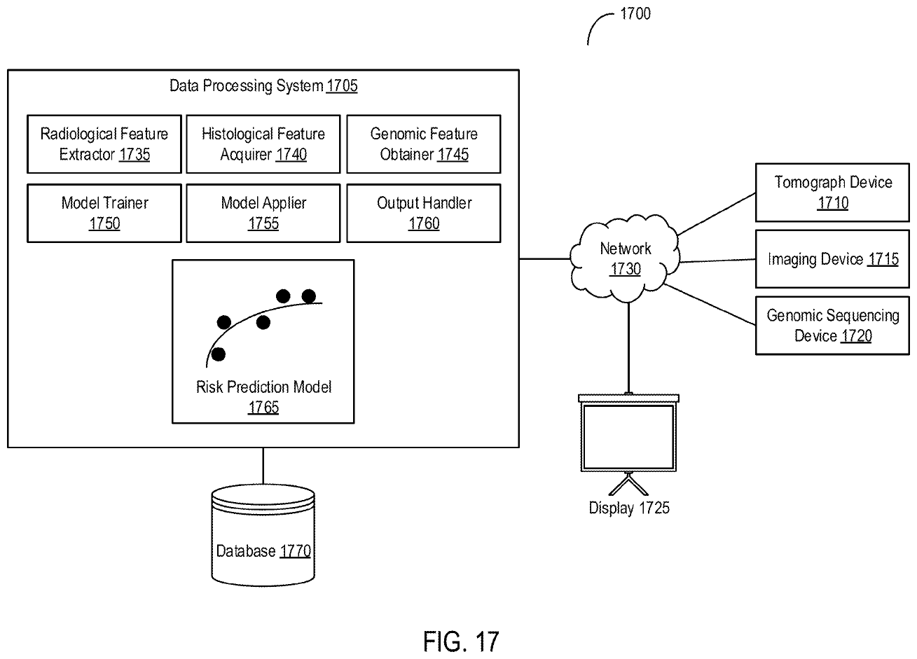

Referring now to FIG. 17, depicted is a block diagram of a system 1700 for determining risk scores using multimodal feature sets. In overview, the system 1700 may include at least one data processing system 1705, at least one tomograph device 1710, at least one imaging device 1715, at least one genomic sequencing device 1720, and at least one display 1725, communicatively coupled via at least one network 1730. The data processing system 1705 may include at least one radiological feature extractor 1735, at least one histological feature acquirer 1740, at least one genomic feature obtainer 1745, at least one model trainer 1750, at least one model applier 1755, and at least one output handler 1760, at least one risk prediction model 1765, and at least one database 1770, among others. Each of the components in the system 1700 as detailed herein may be implemented using hardware (e.g., one or more processors coupled with memory), or a combination of hardware and software as detailed herein in Section C. Each of the components in the system 1700 may implement or execute the functionalities detailed herein, such as those described in Section A.

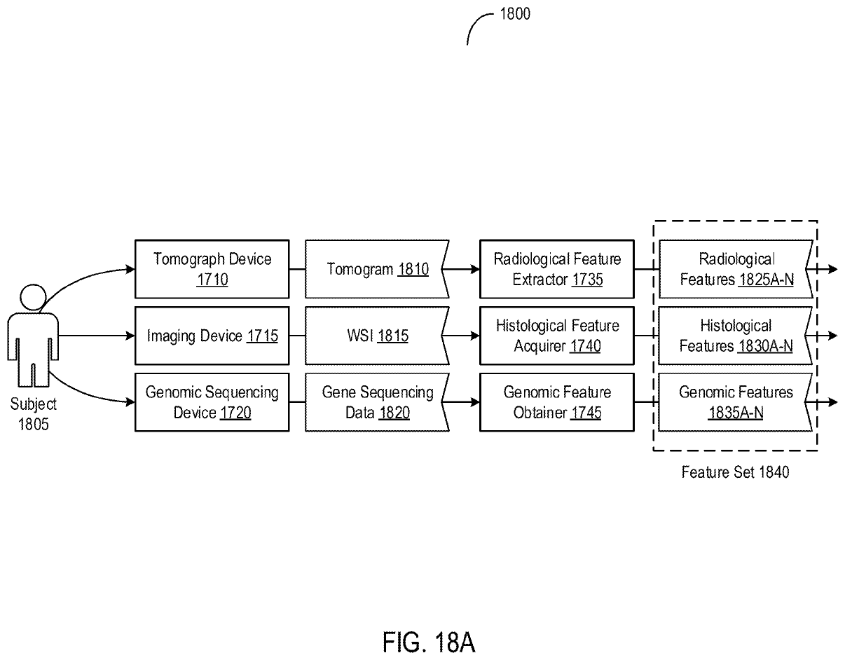

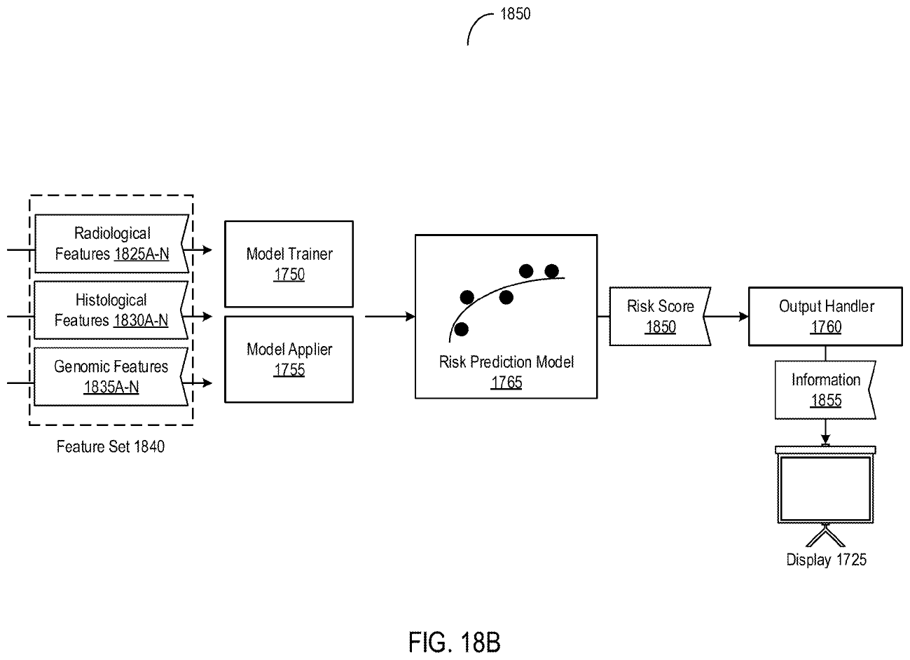

Referring now to FIG. 18A, depicted is a block diagram of a process 1800 of extracting multimodal features in the system 1700 for determining risk scores. The process 1800 may correspond to or include operations in the system 1700 for identifying features in various modalities from subjects. Under the process 1800, one or more devices of the system 1700 may obtain or acquire data in multiple modalities from at least a portion of a subject 1805 (e.g., a human or animal). The subject 1805 may be at risk of a condition, or may be afflicted with the condition. The condition may include, for example, a type of cancer (e.g., breast cancer, bladder cancer, cervical cancer, colorectal cancer, kidney cancer, liver cancer, lung cancer, lymphoma, ovarian cancer, prostate cancer, skin cancer, or thyroid cancer), among others. The subject 1805 may be under evaluation for the progression or deterioration of the condition.

The tomograph device 1710 may produce, output, or otherwise generate at least one tomogram 1810 (sometimes herein referred to generally as a biomedical image or an image) of a section of the subject 1805. For example, the tomogram 1810 may be a scan of the sample corresponding to a tissue of the organ in the subject 1805. The tomogram 1810 may include a set of two-dimensional cross-sections (e.g., a front, a sagittal, a transverse, or an oblique plane) acquired from the three-dimensional volume. The tomogram 1810 may be defined in terms of pixels, in two-dimensions or three-dimensions. In some embodiments, the tomogram 1810 may be part of a video acquired of the sample over time. For example, the tomogram 1810 may correspond to a single frame of the video acquired of the sample over time at a frame rate.

The tomogram 1810 may be acquired using any number of imaging modalities or techniques. For example, the tomogram 1810 may be a tomogram acquired in accordance with a tomographic imaging technique, such as a magnetic resonance imaging (MRI) scanner, a nuclear magnetic resonance (NMR) scanner, X-ray computed tomography (CT) scanner, an ultrasound imaging scanner, and a positron emission tomography (PET) scanner, and a photoacoustic spectroscopy scanner, among others. The tomogram 1810 may be a single instance of acquisition (e.g., X-ray) in accordance with the imaging modality, or may be part of a video (e.g., cardiac MRI) acquired using the imaging modality.

The tomogram 1810 may include or identify at least one at least one region of interest (ROI) (also referred herein as a structure of interest (SOI) or feature of interest (FOI)). The ROI may correspond to an area, section, or part of the tomogram 1810 that corresponds to the presence of the condition in the sample from which the tomogram 1810 is acquired. For example, the ROI may correspond to a portion of the tomogram 1810 depicting a tumorous growth in a CT scan of a brain of a human subject. With the acquisition of the tomogram 1810, the tomograph device 1710 may send, transmit, or otherwise provide the tomogram 1810 to the data processing system 1705. The tomogram 1810 may be in maintained using one or more files in accordance with a format (e.g., single-file or multi-file DICOM format).

The imaging device 1715 may scan, obtain, or otherwise acquire a whole slide image (WSI) 1815 (sometimes herein referred generally as a biomedical image or image) of a tissue sample of the subject 1805. The tissue sample may be obtained from the section of the subject 1805 used to generate the tomogram 1810, or may be taken from another portion associated with the condition within the subject 1805. The WSI 1815 itself may be acquired in accordance with microscopy techniques or a histopathological image preparer, such as using an optical microscope, a confocal microscope, a fluorescence microscope, a phosphorescence microscope, an electron microscope, among others. The WSI 1815 may be for digital pathology of a tissue section in the sample from the subject 1805. The WSI 1815 may be, for example, a histological section with a hematoxylin and eosin (H&E) stain, immunostaining, hemosiderin stain, a Sudan stain, a Schiff stain, a Congo red stain, a Gram stain, a Ziehl-Neelsen stain, a Auramine-rhodamine stain, a trichrome stain, a Silver stain, and Wright's Stain, among others. The WSI 1815 may be maintained using one or more files in accordance with a format (e.g., DICOM whole slide imaging (WSI)).

The WSI 1815 may include one or more regions of interest (ROIs). Each ROI may correspond to areas, sections, or boundaries within the sample WSI 1815 that contain, encompass, or include conditions (e.g., features or objects within the image). The ROIs depicted in the WSI may correspond to areas with cell nuclei. The ROIs of the sample WSI 1815 may correspond to different subtype conditions. For example, when the WSI 1815 is a WSI of the sample tissue, the features may correspond to cell nuclei and the conditions may correspond to various cancer subtypes, such as carcinoma (e.g., adenocarcinoma and squamous cell carcinoma), sarcoma (e.g., osteosarcoma, chondrosarcoma, leiomyosarcoma, rhabdomyosarcoma, mesothelial sarcoma, and fibrosarcoma), myeloma, leukemia (e.g., myelogenous, lymphatic, and polycythemia), lymphoma, and mixed types, among others. Upon generation, the imaging device 1715 may send, transmit, or otherwise provide the WSI 1815 to the data processing system 1705.

The genomic sequencing device 1720 may carry out, execute, or otherwise perform genetic sequencing on a deoxyribonucleic acid (DNA) sample taken from the subject 1805 to generate gene sequencing data 1820. The genetic sequencing carried out may be a high throughput, massively parallel sequencing technique (sometimes herein referred to as next generation sequencing), such as pyrosequencing, Reversible dye-terminator sequencing, SOLiD sequencing, Ion semiconductor sequencing, Helioscope single molecule sequencing, among others. The genetic sequencing may be targeted to find biomarkers associated with or correlated with the condition of the subject 1805. For example, the genomic sequencing device 1720 may perform the hybridization-capture based targeted sequencing to find tumor protein 53 (TP53), BRCA panel (e.g., BRCA1 or BRCA2), G1/S-specific cyclin-E1 (CCNE1), or cyclin-dependent kinase 12 (CDK12), among others. Upon carrying out the sequencing, the genomic sequencing device 1720 may send, transmit, or otherwise provide the gene sequencing data 1820 to the data processing system 1705. The gene sequencing data 1820 may be maintained using one or more files according to a format (e.g., FASTQ, BCL, or VCF formats).

The radiological feature extractor 1735 executing on the data processing system 1705 may generate, determine, or otherwise identify a set of radiological features 1825A-N (hereinafter generally referred to as radiological features 1825) using the tomogram 1810. The radiological feature 1825 may include or identify information derived from the tomogram 1810 of the section associated with the condition in the subject 1805, such as those described in Section A. To identify, the radiological feature extractor 1735 may apply a wavelet transform (e.g., a Coif wavelet transform) on the tomogram 1810. The radiological feature extractor 1735 may calculate, determine, or otherwise generate a matrix from the tomogram 1810 transformed using the wavelet function. The derived matrix for the radiological feature 1825 may, for example, include any one or more of: (i) a gray level co-occurrence matrix (GLCM), gray level dependence matrix (GLDM), (iii) a gray level run length matrix (GLRLM), (vi) a gray level size zone matrix (GLSZM), or (v) a neighboring gray tone difference matrix, among others. The radiological feature 1825 may include any of the features listed in Supplementary Table 4.

The histological feature acquirer 1740 executing on the data processing system 1705 may generate, determine, or otherwise identify a set of histological features 1830A-N (hereinafter generally referred to as histological features 1830) using the WSI 1815. The WSI 1815 may include or identify information derived from the WSI 1815 associated with the condition in the subject 1805. The histological feature acquirer 1740 may use one or more machine learning (ML) models to recognize, detect, or otherwise identify the histological features 1830 from the WSI 1815. The ML models may include, for example: an image segmentation model to determine the ROI within the WSI 1815 associated with the condition; an image classification model to determine the condition type to which to classify sample depicted in the WSI 1815; or an image localization model to determine a portion (e.g., a tile) within the WSI 1815 corresponding to the ROI, among others. The ML model for image segmentation, localization, or classification may be of any architecture, such as a deep learning artificial neural network (ANN), a regression model (e.g., linear or logistic regression), a clustering model (e.g., k-NN clustering or density-based clustering), Naïve Bayesian classifier, a decision tree, a relevance vector machine (RVM), or a support vector machine (SVM), among others.

From applying the image segmentation or localization model, the histological feature acquirer 1740 may determine a portion of the WSI 1815 corresponding to the one or more ROI associated with the condition. The ROIs may correspond to types of tissue or cell nuclei associated with the condition, such as fat, necrosis, stroma lymphocyte, stroma nuclei, stroma, tumor lymphocyte, tumor nuclei, or tumorous tissue, among others. With the determination, the histological feature acquirer 1740 may calculate, determine, or identify one or more properties of the ROIs in the WSI 1815, such as: nuclei cell types within the sample; a mean area (e.g., percentage) of cell nuclei by type within sample; a dimension (e.g., length or width along a given axis) of cell nuclei by type; tissue types within the sample depicted in the WSI 1815; an area (e.g., percentage) of a given tissue type in the sample; a dimension (e.g., diameter, length, or width along a given axis) of the given tissue type in the sample; cells or tissues for a given cancer subtype; an area of the portion of the WSI 1815 corresponding to the cancer subtype; a dimension (e.g., diameter, length, or width along a given axis) of the portion for the cancer subtype; or a statistical measure (e.g., mean, median, standard deviation) in staining (e.g., H&E) indicative of the tissue type or cell nuclei type; among others. In some embodiments, from applying the image classification model, the histological feature acquirer 1740 may determine a classification of the sample in the WSI 1815. The classification may include, for example, a presence or an absence of the condition, such as the type of cancer. The histological feature acquirer 1740 may use the properties of the ROIs in the WSI 1815 and the classification as the histological features 1830. The histological features 1830 may also include any of the features listed in Supplementary Table 5. One or more of the histological features 1830 in the set may be used for training the risk prediction model 1765.

The genomic feature obtainer 1745 executing on the data processing system 1705 may generate, determine, or otherwise identify a set of genomic features 1835A-N using the gene sequencing data 1820. Using the gene sequencing data 1820, the genomic feature obtainer 1745 may identify or determine Homologous recombination deficiency (HRD) or Homologous recombination proficiency (HRP) status of the subject 1805. The determination of the HRD or HRP status may be based on a presence or absence of one or more mutations within the gene sequencing data 1820 for the subject 1805. The genomic feature obtainer 1745 may identify variants associated with HRD DNA damage response (DDR), such as BRCA1, BRCA2, CCNE1, and CDK12, among others. The genomic feature obtainer 1745 may also identify mutational subtypes within the gene sequencing data 1820, such as HRD Deletion (HRD-DEL); HRD-Duplication (HRD-DUP); Foldback Inversion (FBI), and Tandem Duplications (TD), among others. The variants for HRD DDR may have a correspondence with the mutational subtypes, such as: BRCA2 SNVs with HRD-DEL, BRCA1 SNVs with HRD-DUP, CCNE1 CNAs with FBI, and CDK12 SNVs associated with TD, among others.

With the identification, the radiological features 1825, the histological features 1830, and genomic features 1835 may form at least one feature set 1840 (sometimes herein referred to as a multimodal feature set). The feature set 1840 may include one or more features from a variety of modalities, as described herein. The feature set 1840 may be further processed by the data processing system 1705 to evaluate the subject 1805. At least some of the feature sets 1840 together with expected risk scores may be used for training the risk prediction model 1765 as explained below. At least some of the feature sets 1840 may be used at runtime to feed to the risk prediction model 1765 to determine predicted risk scores for subjects 1805.

Referring now to FIG. 18B, depicted a block diagram of a process 1850 of applying risk prediction models to multimodal features. The process 1850 may correspond to or include operations in the system 1700 for establishing a multimodal model and determining risk scores for subjects. Under the process 1850, the model trainer 1750 executing on the data processing system 1705 may initialize or establish the risk prediction model 1765 (sometimes herein referred to as a multimodal or multivariate model). The model trainer 1750 may be invoked to establish the risk prediction model 1765 during training mode. The risk prediction model 1765 may be any machine learning (ML), such as: a regression model (e.g., linear or logistic regression), a clustering model (e.g., k-NN clustering or density-based clustering), Naïve Bayesian classifier, artificial neural network (ANN), a decision tree, a relevance vector machine (RVM), or a support vector machine (SVM), among others. The risk prediction model 1765 may be an instance of the Cox regression models discussed in Section B, such as the multivariate model generated using Algorithm 1. In general, the risk prediction model 1765 may have one or more inputs corresponding to the feature set 1840, one or more outputs for predicted risk scores, and one or more weights relating the inputs and the outputs, among others.

To establish the risk prediction model 1765, the model trainer 1750 may retrieve, receive, or identify training data. The training data may include one or more feature sets 1840 and corresponding expected risk scores, and may be maintained on the database 1770. Each feature set 1840 may identify or include the radiological features 1825, the histological features 1830, and genomic features 1835 for a given sample subject 1805 as discussed above. Each expected risk score may identify or correspond to a likelihood of an occurrence of an event (e.g., survival, hospitalization, injury, pain, treatment, or death) due to the condition in the subject 1805. The expected risk score may be manually created by a clinician (e.g., pathologist) examining the subject 1805 from which the feature set 1840 is obtained. In some embodiments, the training data may include a survival function for each feature set 1840 identifying expected risk scores over a period of time. The period of time may range, for example, from 3 days to 5 years. The model trainer 1750 may set the weights of the risk prediction model 1765 to initial values (e.g., zero or random) when initializing.

In some embodiments, the model trainer 1750 may identify or select features from the feature set 1840 of the training data to apply to the risk prediction model 1765. In selecting for establishing, the model trainer 1750 may identify or select at least one radiological feature 1825 from the set of radiological features 1825. The selection of the at least one radiological feature 1825 may be performed using a model. The model may be any machine learning (ML), such as: a regression model (e.g., linear or logistic regression), a clustering model (e.g., k-NN clustering or density-based clustering), Naïve Bayesian classifier, artificial neural network (ANN), a decision tree, a relevance vector machine (RVM), or a support vector machine (SVM), among others. The model for selecting the radiological features 1825 may be, for example, an instance of the univariate Cox regression model discussed in Section B. The model trainer 1720 may establish the model by updating using the radiological features 1825 and the expected risk scores. The updating may include fitting and pruning the weights of the model for statistical significance of the types of features in the set of radiological features 1825 relative to the expected risk scores.

Upon fitting, the model trainer 1720 may calculate, generate, or otherwise determine a hazard ratio for each type of radiological features 1825 in the set of radiological features 1825 from the model. The model trainer 1720 may also determine, calculate, or otherwise generate a confidence value for each hazard ratio. The hazard ratio may identify or correspond to a degree of effect that the corresponding radiological feature 1825 has on the expected risk score. In general, the lower the hazard ratio, the lower the contributory effect of the radiological feature 1825 has to the expected risk score. Conversely, the higher the hazard ratio, the higher the contributory effect of the radiological feature 1825 has to the expected risk score. Based on the hazard ratio, the model trainer 1720 may select at least one of the radiological features 1825 for training the risk prediction model 1765. For instance, the model trainer 1720 may select the n radiological features 1825 with the highest n hazard ratios with a threshold level of confidence (e.g., 95%).

In addition, the model trainer 1750 may identify or select at least one histological feature 1830 from the set of histological features 1830. The selection of the at least one histological feature 1830 may be performed using a model. The model may be any machine learning (ML), such as: a regression model (e.g., linear or logistic regression), a clustering model (e.g., k-NN clustering or density-based clustering), Naïve Bayesian classifier, artificial neural network (ANN), a decision tree, a relevance vector machine (RVM), or a support vector machine (SVM), among others. The model for selecting the histological features 1830 may be, for example, an instance of the univariate Cox regression model discussed in Section B. The model trainer 1720 may establish the model by updating using the histological features 1830 and the expected risk scores. The updating may include fitting and pruning the weights of the model for statistical significance of the types of features in the set of histological features 1830 relative to the expected risk scores.

Upon fitting, the model trainer 1720 may calculate, generate, or otherwise determine a hazard ratio for each type of histological features 1830 in the set of histological features 1830 from the model. The model trainer 1720 may also determine, calculate, or otherwise generate a confidence value for each hazard ratio. The hazard ratio may identify or correspond to a degree of effect that the corresponding histological feature 1830 has on the expected risk score. In general, the lower the hazard ratio, the lower the contributory effect of the histological feature 1830 has to the expected risk score. Conversely, the higher the hazard ratio, the higher the contributory effect of the histological feature 1830 has to the expected risk score. Based on the hazard ratio and the confidence value, the model trainer 1720 may select at least one of the histological features 1830 for training the risk prediction model 1765. For instance, the model trainer 1720 may select the n histological features 1830 with the highest n hazard ratios with a threshold level of confidence (e.g., 95%). In some embodiments, the model trainer 1750 may use the set of genomic features 1835 for training, without additional selection, as the gene sequencing data 1820 from which the genomic features 1835 are extracted may have been generated using targeted sequencing of DNA from the subject 1805.

From the training data, the model trainer 1750 may identify the feature set 1840 to apply to the risk prediction model 1765. The feature set 1840 may include at least one of the radiological features 1825, at least one of the histological features 1830, and at least one of the genomic features 1835, among others. In some embodiments, the feature set may include the radiological features 1825 and the histological features 1830 selected using the univariate models as discussed above, along with the genomic features 1835. The model trainer 1750 may traverse over the feature sets 1840 of the training data to identify each feature set 1840. To apply, the model trainer 1750 may feed the feature set 1840 into the input of the risk prediction model 1765. Upon feeding, the model trainer 1750 may process the values of the feature set 1840 in accordance with the weights of the risk prediction model 1765 to output a predicted risk score for the feature set 1840. The predicted risk score may be similar to the expected risk score, and may identify or correspond to a likelihood of an occurrence of an event (e.g., survival, hospitalization, injury, pain, treatment, or death) due to the condition in the subject 1805 as calculated using the risk prediction model 1765. In some embodiments, the output may include the survival function identifying predicted risk scores over a period of time.

With the output, the model trainer 1750 may compare the predicted risk scores outputted by the risk prediction model 1765 and the corresponding expected risk scores from the training data. Using the comparison, the model trainer 1750 may update the weights of the risk prediction model 1765. In some embodiments, the model trainer 1750 may calculate, generate, or otherwise determine at least one loss metric (sometimes herein referred to as an error metric) based on the comparison. The loss metric may identify or correspond to a degree of deviation of the predicted risk score from the expected risk score. The loss metric may be calculated in accordance with any number of loss functions, such as a mean squared error (MSE), a mean absolute error (MAE), a hinge loss, a quantile loss, a quadratic loss, a smooth mean absolute loss, and a cross-entropy loss, among others. Using the loss metric, the model trainer 1750 may update the weights of the risk prediction model 1765. The updating (e.g., fitting and pruning) of the weights of the risk prediction model 1765 may be repeated until reaching convergence as defined for the model architecture.

In some embodiments, in updating the risk prediction model 1765, the model trainer 1750 may identify or select one or more features of the feature set 1840 for inputs of the risk prediction model 1765. The selected features may include at least one of the radiological features 1825, at least one of the histological features 1830, and at least one of the genomic features 1835. Upon fitting, the model trainer 1750 may calculate, generate, or otherwise determine a hazard ratio for each type of feature (e.g., the radiological feature 1825, the histological feature 1830, and the genomic feature 1835) in the set of histological features 1830 from the model. The model trainer 1720 may also determine, calculate, or otherwise generate a confidence value for each hazard ratio. The hazard ratio may identify or correspond to a degree of effect that the corresponding feature has on the expected risk score. In general, the lower the hazard ratio, the lower the contributory effect of the feature has to the expected risk score. Conversely, the higher the hazard ratio, the higher the contributory effect of the feature has to the expected risk score. Based on the hazard ratio and the confidence value, the model trainer 1720 may select each of the feature types for training the risk prediction model 1765. For instance, the model trainer 1720 may select the n histological features 1830 and n radiological features 1835 with the highest n hazard ratios with a threshold level of confidence (e.g., 95%) in their respective feature type.

With the establishment of the risk prediction model 1765, the model applier 1755 executing on the data processing system 1705 may receive, retrieve, or otherwise identify the feature set 1840. The feature set 1840 may include at least one of the radiological features 1825, at least one of the histological features 1830, and at least one of the genomic features 1835. The feature set 1840 may be newly acquired, and differ from the feature sets 1840 of the training data as described above. Under runtime mode, the type of radiological features 1825, histological features 1830, and genomic features 1835 may correspond to those selected during training of the risk prediction model 1765. Upon the identification, the model applier 1755 may feed the feature set 1840 into the input of the risk prediction model 1765.

In feeding, the model applier 1755 may process the values of the feature set 1840 in accordance with the weights of the risk prediction model 1765 to output at least one predicted risk score 1850 for the feature set 1840. The predicted risk score 1850 may identify or correspond to a likelihood of an occurrence of an event (e.g., hospitalization, injury, pain, treatment, or death) due to the condition in the subject 1805 as calculated using the risk prediction model 1765. In some embodiments, the model applier 1755 may calculate, determine, or otherwise generate a survival function identifying predicted risk scores 1765 over a period of time using the risk prediction model 1765.

With the generation, the output handler 1760 executing on the data processing system 1705 may generate an association between the predicted risk score 1765 (or the survival function) and the feature set 1740 using one or more data structures, such as a linked list, a tree, an array, a table, a matrix, a stack, a queue, or a heap, among others. In some embodiments, the association may be among the predicted risk scores 1765, the subject 1805 (e.g., using an anonymized identifier), data used to generate the feature set 1840 (e.g., the tomogram 1810, the WSI 1815, and gene sequencing data 1820) and the feature set 1840. The data structures for the association may be stored and maintained on the database 1770.

In some embodiments, the output handler 1760 may categorize, assign, or otherwise classify the subject 1805 into one of a set of risk level groups based on the predicted risk score 1765. The groups may be used to classify subjects 1805 by predicted risk score 1765. For example, one group may correspond to low risk of a particular cancer and another group may correspond to high risk for the same type of cancer. To classify, the output handler 1760 may compare the predicted risk score 1765 for the subject 1805 with a threshold for each risk level group. The threshold may delineate or define a value (or range) for the predicted risk scores 1765 above which the subject 1805 is to be classified into the associated risk level group. When the predicted risk score 1765 satisfies the threshold for at least one risk level group, the output handler 1760 may assign the subject 1805 (e.g., using the anonymized identifier) to the associated risk level group.

In some embodiments, the output handler 1760 may generate information 1855 based on the predicted risk score 1850 (or the association). The information 1855 may include instructions for rendering, displaying, or otherwise presenting the predicted risk score 1850, along with the identifier for the subject 1805 and the feature set 1840, among others. Upon generation, the output handler 1760 may send, transmit, or otherwise provide the information 1855 to the display 1725 (or a computing device coupled with the display 1725). The provision of the information 1855 may be in response to a request from a user of the data processing system 1705 or the computing device. The display 1725 may render, display, or otherwise present the information 1855, such as the predicted risk score 1850, the feature set 1840, and the identifier for the subject 1805, among others. For instance, the display 1725 may display, render, or otherwise present the information 1855 via a graphical user interface of an application to display predicted risk score 1850 and the classification into a risk level, adjacent to the tomogram 1810, the WSI 1815, and the gene sequencing data 1820, among others.

In this manner, the data processing system 1705 may be able to process features from various modalities (e.g., tomogram 1810, the WSI 1815, and the gene sequencing data 1820) to more accurate generate the predicted risk scores 1850. The features from various modalities may be obtained from various portions of the treatment process of the subject 1805, thereby enriching the types of data used to apply to the risk prediction model 1765. By outputting more accurate risk scores 1850, the data processing system 1705 may save computing resources (e.g., processor and memory consumption) that would have been exhausted from providing inaccurate and thus less useful risk scores.

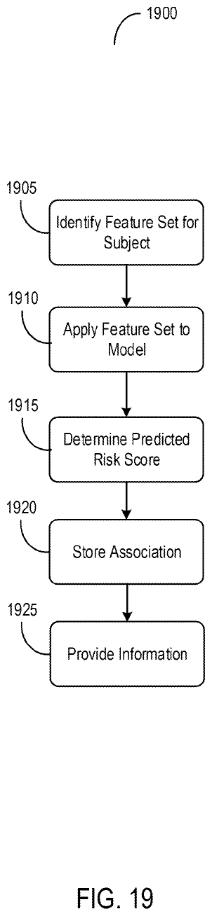

Referring now to FIG. 19, depicted is a flow diagram of a method 1900 of determining risk scores using multimodal feature sets. The method 1900 may be performed by or implementing using the system 1700 described herein in conjunction with FIGS. 17-18B or the system 2000 as described herein in conjunction with Section C. Under the method 1900, a computing system (e.g., the data processing system 1705) may identify a feature set (e.g., the feature set 1840 including the radiological feature 1825, the histological feature 1830, and the genomic feature 1835) (1905). The computing system may apply the feature set to a model (e.g., the risk prediction model 1765) (1910). The computing system may determine a predicted risk score (e.g., the predicted risk score 1850) from the application of the model (1915). The computing system may store an association between the predicted risk score and a subject (e.g., the subject 1805) (1920). The computing system may provide information (e.g., the information 1855) based on the predicted risk score (1925).

### C. Computing and Network Environment

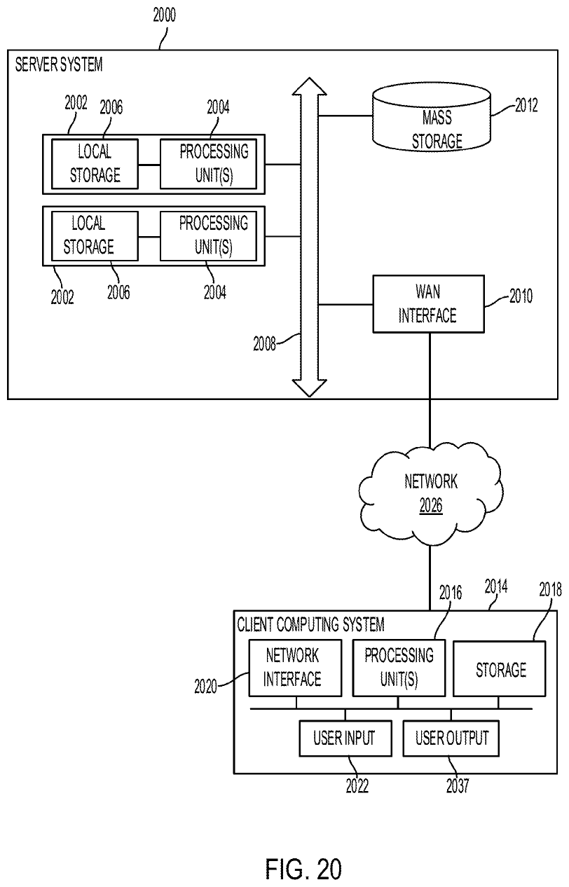

Various operations described herein can be implemented on computer systems. FIG. 20 shows a simplified block diagram of a representative server system 2000, client computer system 2014, and network 2026 usable to implement certain embodiments of the present disclosure. In various embodiments, server system 2000 or similar systems can implement services or servers described herein or portions thereof. Client computer system 2014 or similar systems can implement clients described herein. The systems 1700 described herein can be similar to the server system 2000. Server system 2000 can have a modular design that incorporates a number of modules 2002 (e.g., blades in a blade server embodiment); while two modules 2002 are shown, any number can be provided. Each module 2002 can include processing unit(s) 2004 and local storage 2006.

Processing unit(s) 2004 can include a single processor, which can have one or more cores, or multiple processors. In some embodiments, processing unit(s) 2004 can include a general-purpose primary processor as well as one or more special-purpose co-processors such as graphics processors, digital signal processors, or the like. In some embodiments, some or all processing units 2004 can be implemented using customized circuits, such as application specific integrated circuits (ASICs) or field programmable gate arrays (FPGAs). In some embodiments, such integrated circuits execute instructions that are stored on the circuit itself. In other embodiments, processing unit(s) 2004 can execute instructions stored in local storage 2006. Any type of processors in any combination can be included in processing unit(s) 2004.

Local storage 2006 can include volatile storage media (e.g., DRAM, SRAM, SDRAM, or the like) and/or non-volatile storage media (e.g., magnetic or optical disk, flash memory, or the like). Storage media incorporated in local storage 2006 can be fixed, removable, or upgradeable as desired. Local storage 2006 can be physically or logically divided into various subunits such as a system memory, a read-only memory (ROM), and a permanent storage device. The system memory can be a read-and-write memory device or a volatile read-and-write memory, such as dynamic random-access memory. The system memory can store some or all of the instructions and data that processing unit(s) 2004 need at runtime. The ROM can store static data and instructions that are needed by processing unit(s) 2004. The permanent storage device can be a non-volatile read-and-write memory device that can store instructions and data even when module 2002 is powered down. The term “storage medium” as used herein includes any medium in which data can be stored indefinitely (subject to overwriting, electrical disturbance, power loss, or the like) and does not include carrier waves and transitory electronic signals propagating wirelessly or over wired connections.

In some embodiments, local storage 2006 can store one or more software programs to be executed by processing unit(s) 2004, such as an operating system and/or programs implementing various server functions such as functions of the systems 1700 or any other system described herein, or any other server(s) associated with systems 1700 or any other system described herein.

“Software” refers generally to sequences of instructions that, when executed by processing unit(s) 2004, cause server system 2000 (or portions thereof) to perform various operations, thus defining one or more specific machine embodiments that execute and perform the operations of the software programs. The instructions can be stored as firmware residing in read-only memory and/or program code stored in non-volatile storage media that can be read into volatile working memory for execution by processing unit(s) 2004. Software can be implemented as a single program or a collection of separate programs or program modules that interact as desired. From local storage 2006 (or non-local storage described below), processing unit(s) 2004 can retrieve program instructions to execute and data to process in order to execute various operations described above.

In some server systems 2000, multiple modules 2002 can be interconnected via a bus or other interconnect 2008, forming a local area network that supports communication between modules 2002 and other components of server system 2000. Interconnect 2008 can be implemented using various technologies including server racks, hubs, routers, etc.

A wide area network (WAN) interface 2010 can provide data communication capability between the local area network (interconnect 2008) and the network 2026, such as the Internet. Technologies can be used, including wired (e.g., Ethernet, IEEE 802.3 standards) and/or wireless technologies (e.g., Wi-Fi, IEEE 802.11 standards).

In some embodiments, local storage 2006 is intended to provide working memory for processing unit(s) 2004, providing fast access to programs and/or data to be processed while reducing traffic on interconnect 2008. Storage for larger quantities of data can be provided on the local area network by one or more mass storage subsystems 2012 that can be connected to interconnect 2008. Mass storage subsystem 2012 can be based on magnetic, optical, semiconductor, or other data storage media. Direct attached storage, storage area networks, network-attached storage, and the like can be used. Any data stores or other collections of data described herein as being produced, consumed, or maintained by a service or server can be stored in mass storage subsystem 2012. In some embodiments, additional data storage resources may be accessible via WAN interface 2010 (potentially with increased latency).

Server system 2000 can operate in response to requests received via WAN interface 2010. For example, one of the modules 2002 can implement a supervisory function and assign discrete tasks to other modules 2002 in response to received requests. Work allocation techniques can be used. As requests are processed, results can be returned to the requester via WAN interface 2010. Such operation can generally be automated. Further, in some embodiments, WAN interface 2010 can connect multiple server systems 2000 to each other, providing scalable systems capable of managing high volumes of activity. Other techniques for managing server systems and server farms (collections of server systems that cooperate) can be used, including dynamic resource allocation and reallocation.

Server system 2000 can interact with various user-owned or user-operated devices via a wide-area network such as the Internet. An example of a user-operated device is shown in FIG. 20 as client computing system 2014. Client computing system 2014 can be implemented, for example, as a consumer device such as a smartphone, other mobile phone, tablet computer, wearable computing device (e.g., smart watch, eyeglasses), desktop computer, laptop computer, and so on.

For example, client computing system 2014 can communicate via WAN interface 2010. Client computing system 2014 can include computer components such as processing unit(s) 2016, storage device 2018, network interface 2020, user input device 2022, and user output device 2037. Client computing system 2014 can be a computing device implemented in a variety of form factors, such as a desktop computer, laptop computer, tablet computer, smartphone, other mobile computing device, wearable computing device, or the like.

Processing unit(s) 2016 and storage device 2018 can be similar to processing unit(s) 2004 and local storage 2006 described above. Suitable devices can be selected based on the demands to be placed on client computing system 2014; for example, client computing system 2014 can be implemented as a “thin” client with limited processing capability or as a high-powered computing device. Client computing system 2014 can be provisioned with program code executable by processing unit(s) 2016 to enable various interactions with server system 2000.

Network interface 2020 can provide a connection to the network 2026, such as a wide area network (e.g., the Internet) to which WAN interface 2010 of server system 2000 is also connected. In various embodiments, network interface 2020 can include a wired interface (e.g., Ethernet) and/or a wireless interface implementing various RF data communication standards such as Wi-Fi, Bluetooth, or cellular data network standards (e.g., 3G, 4G, LTE, etc.).

User input device 2022 can include any device (or devices) via which a user can provide signals to client computing system 2014; client computing system 2014 can interpret the signals as indicative of particular user requests or information. In various embodiments, user input device 2022 can include any or all of a keyboard, touch pad, touch screen, mouse or other pointing device, scroll wheel, click wheel, dial, button, switch, keypad, microphone, and so on.

User output device 2037 can include any device via which client computing system 2014 can provide information to a user. For example, user output device 2037 can include display-to-display images generated by or delivered to client computing system 2014. The display can incorporate various image generation technologies, e.g., a liquid crystal display (LCD), light-emitting diode (LED) including organic light-emitting diodes (OLED), projection system, cathode ray tube (CRT), or the like, together with supporting electronics (e.g., digital-to-analog or analog-to-digital converters, signal processors, or the like). Some embodiments can include a device such as a touchscreen that function as both input and output device. In some embodiments, other user output devices 2037 can be provided in addition to or instead of a display. Examples include indicator lights, speakers, tactile “display” devices, printers, and so on.

Some embodiments include electronic components, such as microprocessors, storage, and memory that store computer program instructions in a computer readable storage medium. Many of the features described in this specification can be implemented as processes that are specified as a set of program instructions encoded on a computer readable storage medium. When these program instructions are executed by one or more processing units, they cause the processing unit(s) to perform various operations indicated in the program instructions. Examples of program instructions or computer code include machine code, such as is produced by a compiler, and files including higher-level code that are executed by a computer, an electronic component, or a microprocessor using an interpreter. Through suitable programming, processing unit(s) 2004 and 2016 can provide various functionality for server system 2000 and client computing system 2014, including any of the functionality described herein as being performed by a server or client, or other functionality.

It will be appreciated that server system 2000 and client computing system 2014 are illustrative and that variations and modifications are possible. Computer systems used in connection with embodiments of the present disclosure can have other capabilities not specifically described here. Further, while server system 2000 and client computing system 2014 are described with reference to particular blocks, it is to be understood that these blocks are defined for convenience of description and are not intended to imply a particular physical arrangement of component parts. For instance, different blocks can be but need not be located in the same facility, in the same server rack, or on the same motherboard. Further, the blocks need not correspond to physically distinct components. Blocks can be configured to perform various operations, e.g., by programming a processor or providing appropriate control circuitry, and various blocks might or might not be reconfigurable depending on how the initial configuration is obtained. Embodiments of the present disclosure can be realized in a variety of apparatus including electronic devices implemented using any combination of circuitry and software.

While the disclosure has been described with respect to specific embodiments, one skilled in the art will recognize that numerous modifications are possible. Embodiments of the disclosure can be realized using a variety of computer systems and communication technologies, including, but not limited to, specific examples described herein. Embodiments of the present disclosure can be realized using any combination of dedicated components and/or programmable processors and/or other programmable devices. The various processes described herein can be implemented on the same processor or different processors in any combination. Where components are described as being configured to perform certain operations, such configuration can be accomplished; e.g., by designing electronic circuits to perform the operation, by programming programmable electronic circuits (such as microprocessors) to perform the operation, or any combination thereof. Further, while the embodiments described above may refer to specific hardware and software components, those skilled in the art will appreciate that different combinations of hardware and/or software components may also be used and that particular operations described as being implemented in hardware might also be implemented in software or vice versa.

Computer programs incorporating various features of the present disclosure may be encoded and stored on various computer readable storage media; suitable media includes magnetic disk or tape, optical storage media such as compact disk (CD) or digital versatile disk (DVD), flash memory, and other non-transitory media. Computer readable media encoded with the program code may be packaged with a compatible electronic device, or the program code may be provided separately from electronic devices (e.g., via Internet download or as a separately packaged computer-readable storage medium).

Thus, although the disclosure has been described with respect to specific embodiments, it will be appreciated that the disclosure is intended to cover all modifications and equivalents within the scope of the following claims.

What is claimed is:

1. A method of determining risk stratification for subjects, comprising:
identifying, by a computing system, a first feature set for a first subject at risk of a condition, the first feature set comprising:
(i) a first radiological feature derived from a tomogram of a section associated with the condition within the first subject;
(ii) a first histologic feature acquired using a whole slide image of a sample having the condition from the first subject, and
(iii) a first genomic feature obtained from gene sequencing of the first subject for genes associated with the condition;
applying, by the computing system, the first feature set to a model, wherein the model is established using a plurality of second feature sets and a plurality of expected risk scores for a corresponding plurality of second subjects;
determining, by the computing system, from applying the first feature set to the model, a predicted risk score of the condition for the first subject; and
storing, by the computing system, using one or more data structures, an association between the predicted risk score and the first feature set for the first subject.

2. The method of claim 1, further comprising classifying, by the computing system, the first subject into one of a plurality of risk level groups based on a comparison between the predicted risk score indicating a likelihood of an occurrence of an event due to the condition in the first subject and a threshold for each of the plurality of risk level groups.

3. The method of claim 1, further comprising establishing, by the computing system, the model comprising a multivariate model using one or more features selected from the plurality of second feature set using one or more corresponding univariate models.

4. The method of claim 1, wherein determining the predicted risk score further comprises determining a survival function identifying the predicted risk score for the first subject over a period of time.

5. The method of claim 1, wherein identifying the first feature set further comprises selecting, from a plurality of radiological features, the first radiological feature based on a hazard ratio of each of the plurality of radiological features determined using a univariate model for radiological features.

6. The method of claim 1, wherein identifying the first feature set further comprises selecting, from a plurality of histological features, the first histological feature based on a hazard radio of each of the plurality of histological features determined using a univariate model for histological features.

7. The method of claim 1, wherein the first radiological feature is derived from the tomogram using a Coif-wavelet transform, and comprises at least one of: (i) a gray level co-occurrence matrix (GLCM), (ii) gray level dependence matrix (GLDM), (iii) a gray level run length matrix (GLRLM), (vi) a gray level size zone matrix (GLSZM), or (v) a neighboring gray tone difference matrix.

8. The method of claim 1, wherein the first histologic feature further comprises at least one of: (i) a tissue type of the sample from which the whole slide image is derived, (ii) an area of cell nuclei corresponding to the condition within the sample, or (iii) a length of a portion of the sample corresponding to the tissue type.

9. The method of claim 1, wherein the first genomic feature identifies a status of Homologous recombination deficiency (HRD) or Homologous recombination proficiency (HRP) in the first subject, the status determined using at least one of: (i) variants in genes associated with HRD DNA damage response or (ii) subtypes for disjoint tandem duplicator and foldback inversion mutations.

10. The method of claim 1, further comprising providing, by the computing system, information based on the association between the predicted risk score and the first feature set for the first subject.

11. A system for determining risk stratification for subjects, comprising:
a computing system having one or more processors coupled with memory, configured to:
identify a first feature set for a first subject at risk of a condition, the first feature set comprising:
(i) a first radiological feature derived from a tomogram of a section associated with the condition within the first subject;
(ii) a first histologic feature acquired using a whole slide image of a sample having the condition from the first subject, and
(iii) a first genomic feature obtained from gene sequencing of the first subject for genes associated with the condition;
apply the first feature set to a model, wherein the model is established using a plurality of second feature sets and a plurality of expected risk scores for a corresponding plurality of second subjects;
determine, from applying the first feature set to the model, a predicted risk score of the condition for the first subject; and
store, using one or more data structures, an association between the predicted risk score and the first feature set for the first subject.

12. The system of claim 11, wherein the computing system is further configured to classify the first subject into one of a plurality of risk level groups based on a comparison between the predicted risk score indicating a likelihood of an occurrence of an event due to the condition in the first subject and a threshold for each of the plurality of risk level groups.

13. The system of claim 11, wherein the computing system is further configured to establish the model comprising a multivariate model using one or more features selected from the plurality of second feature set using one or more corresponding univariate models.

14. The system of claim 11, wherein the computing system is further configured to determine a survival function identifying the predicted risk score for the first subject over a period of time.

15. The system of claim 11, wherein the computing system is further configured to select, from a plurality of radiological features, the first radiological feature based on a hazard ratio of each of the plurality of radiological features determined using a univariate model for radiological features.

16. The system of claim 11, wherein the computing system is further configured to select, from a plurality of histological features, the first histological feature based on a hazard radio of each of the plurality of histological features determined using a univariate model for histological features.

17. The system of claim 11, wherein the first radiological feature is derived from the tomogram using a Coif-wavelet transform, and comprises at least one of: (i) a gray level co-occurrence matrix (GLCM), (ii) gray level dependence matrix (GLDM), (iii) a gray level run length matrix (GLRLM), (vi) a gray level size zone matrix (GLSZM), or (v) a neighboring gray tone difference matrix.

18. The system of claim 11, wherein the first histologic feature further comprises at least one of: (i) a tissue type of the sample from which the whole slide image is derived, (ii) an area of cell nuclei corresponding to the condition within the sample, or (iii) a length of a portion of the sample corresponding to the tissue type.

19. The system of claim 11, wherein the first genomic feature identifies a status of Homologous recombination deficiency (HRD) or Homologous recombination proficiency (HRP) in the first subject, the status determined using at least one of: (i) variants in genes associated with HRD DNA damage response or (ii) subtypes for disjoint tandem duplicator and foldback inversion mutations.

20. The system of claim 11, wherein the computing system is further configured to provide information based on the association between the predicted risk score and the first feature set for the first subject.
